(19) United States
(12) Patent Application Publication
(10) Pub. No.: US 2007/0129288 A1
(43) Pub. Date: Jun. 7, 2007

(54) Antibacterial amide macrocycles

(75) Inventors: Thomas Lampe, Dusseldorf (DE); Isabelle Adelt, Dusseldorf (DE); Dieter Beyer, Wuppertal (DE); Nina Brunner, Essen (DE); Rainer Endermann, Wuppertal (DE); Kerstin Ehlert, Velbert (DE); Heinz-Peter Kroll, Wuppertal (DE); Franz von Nussbaum, Dusseldorf (DE); Siegfried Raddatz, Koln (DE); Joachim Rudolph, Guilford, CT (US); Guido Schiffer, Wuppertal (DE); Andreas Schumacher, Efringen-Kirchen (DE); Yolanda Canco-Grande, Hilden (DE); Martin Michels, Solingen (DE); Stefan Weigand, Wuppertal (DE)

Correspondence Address: JEFFREY M. GREENMAN, BAYER PHARMACEUTICALS CORPORATION, 400 MORGAN LANE, WEST HAVEN, CT, 06516, US

(73) Assignee: BAYER HEALTHCARE AG, LEVERKUSEN (DE)

(21) Appl. No.: 10/518,600

(22) PCT Filed: Jun. 10, 2003
(86) PCT No.: PCT/EP03/06078

§ 371 Date: Dec. 23, 2005

(30) Foreign Application Priority Data
Jun. 17, 2002 (DE) 102 26 921.1

(51) Int. Cl. A61K 38/12; C07K 5/12

(52) U.S. Cl. 514/9; 530/317

(57) ABSTRACT

The invention relates to antibacterial amide macrocycles, to methods for the production thereof, and to the use of the same for producing pharmaceuticals for the treatment and/or prophylaxis of illness, especially bacterial infections.

The invention relates to antibacterial amide macrocycles and processes for their preparation, and to their use for producing medicaments for the treatment and/or prophylaxis of diseases, in particular of bacterial infections.

U.S. Pat. No. 3,452,136, thesis of R. U. Meyer, Stuttgart University, Germany 1991, thesis of V. Leitenberger, Stuttgart University, Germany 1991, Synthesis (1992), (10), 1025-30, J. Chem. Soc., Perkin Trans. 1 (1992), (1), 123-30, J. Chem. Soc., Chem. Commun. (1991), (10), 744, Synthesis (1991), (5), 409-13, J. Chem. Soc., Chem. Commun. (1991), (5), 275-7, J. Antibiot. (1985), 38(11), 1462-8, J. Antibiot. (1985), 38(11), 1453-61, describe the natural product biphenomycin B as having antibacterial activity. The structure of biphenomycin B corresponds to formula (I) hereinafter, where R¹, R², R³, R⁴, R⁷, R⁸ and R⁹ are hydrogen, R³ is 3-amino-2-hydroxyprop-1-yl, and C(O)NR⁵R⁶ is replaced by carboxyl (COOH). Some steps in the synthesis of biphenomycin B are described in Synlett (2003), 4, 522-525.

Chirality (1995), 7(4), 181-92, J. Antibiot. (1991), 44(6), 674-7, J. Am. Chem. Soc. (1989), 111(19), 7323-7, J. Am. Chem. Soc. (1989), 111(19), 7328-33, J. Org. Chem. (1987), 52(24), 5435-7, Anal. Biochem. (1987), 165(1), 108-13, J. Org. Chem. (1985), 50(8), 1341-2, J. Antibiot. (1993), 46(3), C-2, J. Antibiot. (1993), 46(1), 135-40, Synthesis (1992), (12), 1248-54, Appl. Environ. Microbiol. (1992), 58(12), 3879-8, J. Chem. Soc., Chem. Commun. (1992), (13), 951-3 describe a structurally related natural product, biphenomycin A, which has a further substitution with a hydroxy group on the macrocycle.

The natural products do not in terms of their properties comply with the requirements for antibacterial medicaments. Although structurally different agents with antibacterial activity are available on the market, the development of resistance is a regular possibility. Novel agents for good and more effective therapy are therefore desirable.

One object of the present invention is therefore to provide novel and alternative compounds with the same or improved antibacterial effect for the treatment of bacterial diseases in humans and animals.

It has surprisingly been found that derivatives of these natural products in which the carboxyl group of the natural product is replaced by an amide group have antibacterial activity.

The invention relates to compounds of the formula 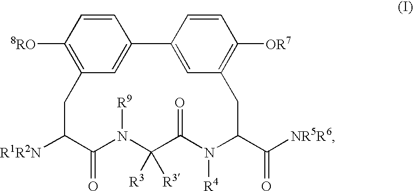
in which
R¹ is hydrogen, alkyl, aryl, heteroaryl, heterocyclyl, alkylcarbonyl, arylcarbonyl, heterocyclylcarbonyl, heteroarylcarbonyl, alkoxycarbonyl, aminocarbonyl, alkylaminocarbonyl, dialkylaminocarbonyl, alkylsulfonyl, arylsulfonyl, heterocyclylsulfonyl, heteroarylsulfonyl or a carbonyl-linked amino acid residue,
   where R¹ apart from hydrogen may be substituted by 0, 1, 2 or 3 substituents R¹⁻¹, where the substituents R¹⁻¹ are selected independently of one another from the group consisting of halogen, alkyl, trifluoromethyl, trifluoromethoxy, nitro, cyano, amino, alkylamino, dialkylamino, cycloalkyl, aryl, heteroaryl, heterocyclyl, hydroxy, alkoxy and carboxyl,
R² is hydrogen or alkyl,
   where R² apart from hydrogen may be substituted by 0, 1, 2 or 3 substituents R²⁻¹, where the substituents R²⁻¹ are selected independently of one another from the group consisting of halogen, amino, alkylamino and dialkylamino,
      or
R¹ and R² together with the nitrogen atom to which they are bonded form a heterocycle which may be substituted by 0, 1 or 2 substituents R¹⁻², where the substituents R¹⁻² are selected independently of one another from the group consisting of halogen, trifluoromethyl, amino, alkylamino, dialkylamino, cycloalkyl, aryl, heteroaryl, heterocyclyl, hydroxy, alkoxy, carboxyl, alkoxycarbonyl and aminocarbonyl,
R³ is hydrogen, alkyl or the side group of an amino acid, in which alkyl may be substituted by 0, 1, 2 or 3 substituents R³⁻¹, where the substituents R³⁻¹ are selected independently of one another from the group consisting of trifluoromethyl, nitro, amino, alkylamino, dialkylamino, cycloalkyl, aryl, heteroaryl, heterocyclyl, hydroxy, alkoxy, carboxyl, alkoxycarbonyl, aminocarbonyl, alkylaminocarbonyl, dialkylaminocarbonyl, guanidino and amidino,
   in which cycloalkyl, aryl, heteroaryl and heterocyclyl may be substituted by 0, 1 or 2 substituents R³⁻², where the substituents R³⁻² are selected independently of one another from the group consisting of halogen, alkyl, trifluoromethyl and amino,
   and in which free amino groups in the side group of the amino acid may be substituted by alkyl, alkenyl, alkynyl, cycloalkyl, aryl, heteroaryl, heterocyclyl, alkylcarbonyl, arylcarbonyl, heteroarylcarbonyl, heterocyclylcarbonyl, alkoxycarbonyl, aminocarbonyl, alkylaminocarbonyl, dialkylaminocarbonyl, arylaminocarbonyl, alkylsulfonyl, arylsulfonyl, heterocyclylsulfonyl or heteroarylsulfonyl,
R^{3′} is hydrogen, C₁-C₆-alkyl or C₃-C₈-cycloalkyl,
R⁴ is hydrogen, C₁-C₆-alkyl or C₃-C₈-cycloalkyl,
R⁵ is hydrogen, alkyl, alkenyl, cycloalkyl, aryl, heteroaryl, heterocyclyl or an amine-linked amino acid residue,
   where R⁵ may be substituted by 0, 1, 2 or 3 substituents R⁵⁻¹, where the substituents R⁵⁻¹ are selected independently of one another from the group consisting of halogen, alkyl, trifluoromethyl, trifluoromethoxy, nitro, cyano, amino, alkylamino, dialkylamino, cycloalkyl, aryl, heteroaryl, heterocyclyl, hydroxy, alkoxy, carboxyl, alkoxycarbonyl, aminocarbonyl, alkylaminocarbonyl, dialkylaminocarbonyl, aminosulfonyl, alkylaminosulfonyl, dialkylaminosulfonyl, arylaminosulfonyl, heterocyclylaminosulfonyl, heteroarylaminosulfonyl, aminocarbonylamino, hydroxycarbonylamino and alkoxycarbonylamino,
      in which alkyl, alkylamino, dialkylamino, cycloalkyl, aryl, heteroaryl and heterocyclyl may be substituted by 0, 1, 2 or 3 substituents R⁵⁻², where the substituents R⁵⁻² are selected independently of one another from the group consisting of hydroxy, amino, carboxyl and aminocarbonyl,
R⁶ is hydrogen, alkyl or cycloalkyl,
   or
R⁵ and R⁶ together with the nitrogen atom to which they are bonded form a heterocycle which may be substituted by 0, 1, 2 or 3 substituents R⁵⁻⁶, where the substituents R⁵⁻⁶ are selected independently of one another from the group consisting of halogen, alkyl, trifluoromethyl, nitro, amino, alkylamino, dialkylamino, cycloalkyl, aryl, halogenated aryl, heteroaryl, heterocyclyl, hydroxy, alkoxy, carboxyl, alkylcarbonyl, alkoxycarbonyl, aminocarbonyl, alkylaminocarbonyl and dialkylaminocarbonyl,
R⁷ is hydrogen, C₁-C₆-alkyl, alkylcarbonyl or C₃-C₈-cycloalkyl,
R⁸ is hydrogen or C₁-C₆-alkyl, and
R⁹ is hydrogen or C₁-C₆-alkyl,
   and the salts thereof, or the solvates thereof and the solvates of the salts thereof.

Compounds of the invention are the compounds of the formula (I) and the salts, solvates and solvates of the salts thereof, the compounds which are encompassed by formula (I) and are of the formula (I′) mentioned below, and the salts, solvates, and solvates of the salts thereof, and the compounds which are encompassed by formula (I) and/or (I′) and are mentioned below as exemplary embodiment(s), and the salts, solvates and solvates of the salts thereof, where the compounds which are encompassed by formula (I) and/or (I′) and are mentioned below are not already salts, solvates and solvates of the salts.

The compounds of the invention may, depending on their structure, exist in stereoisomeric forms (enantiomers, diastereomers). The invention therefore relates to the enantiomers or diastereomers and respective mixtures thereof. The stereoisomerically pure constituents can be isolated from such mixtures of enantiomers and/or diastereomers by known processes such as chromatography on a chiral phase or crystallization using chiral amines or chiral acids.

The invention also relates to tautomers of the compounds, depending on the structure of the compounds.

Salts preferred for the purposes of the invention are physiologically acceptable salts of the compounds of the invention.

Physiologically acceptable salts of the compounds (I) include acid addition salts of mineral acids, carboxylic acids and sulfonic acids, e.g. salts of hydrochloric acid, hydrobromic acid, sulfuric acid, phosphoric acid, methanesulfonic acid, ethanesulfonic acid, toluenesulfonic acid, benzenesulfonic acid, naphthalenedisulfonic acid, acetic acid, propionic acid, lactic acid, tartaric acid, malic acid, citric acid, fumaric acid, maleic acid, trifluoroacetic acid and benzoic acid.

Physiologically acceptable salts of the compounds (I) also include salts of conventional bases such as, by way of example and preferably, alkali metal salts (e.g. sodium and potassium salts), alkaline earth metal salts (e.g. calcium and magnesium salts) and ammonium salts derived from ammonia or organic amines having 1 to 16 C atoms, such as, by way of example and preferably, ethylamine, diethylamine, triethylamine, ethyldiisopropylamine, monoethanolamine, diethanolamine, triethanolamine, dicyclohexylamine, dimethylaminoethanol, procaine, dibenzylamine, N-methylmorpholine, dihydroabietylamine, arginine, lysine, ethylenediamine and methylpiperidine.

Solvates refer for the purposes of the invention to those forms of the compounds which form a complex in the solid or liquid state by coordination with solvent molecules. Hydrates are a special form of solvates in which the coordination takes place with water.

For the purposes of the present invention, the substituents have the following meaning, unless specified otherwise:

Alkyl and the alkyl moieties in substituents such as alkoxy, mono- and dialkylamino, alkylsulfonyl include linear and branched alkyl, e.g. C₁-C₁₂-, in particular C₁-C₆- and C₁-C₄-alkyl.

C₁-C₆-Alkyl includes methyl, ethyl, n- and i-propyl, n-, i-, sec- and tert-butyl, n-pentyl, isopentyl, neopentyl, hexyl,

C₁-C₄-Alkyl includes methyl, ethyl, n- and i-propyl, n-, i-, sec- and tert-butyl,

Alkylcarbonyl is for the purposes of the invention preferably a straight-chain or branched alkyl radical having 1 to 6 or 1 to 4 carbon atoms. Those which may be mentioned by way of example and preferably are: methylcarbonyl, ethylcarbonyl, n-propylcarbonyl, isopropylcarbonyl and t-butylcarbonyl.

Alkenyl includes linear and branched C₂-C₁₂-, in particular C₂-C₆- and C₂-C₄-alkenyl, such as, for example, vinyl, allyl, prop-1-en-1-yl, isopropenyl, but-1-enyl, but-2-enyl, buta-1.2-dienyl, buta-1.3-dienyl.

Alkynyl includes linear and branched C₂-C₁₂-, in particular C₂-C₆- and C₂-C₄-alkynyl, such as, for example, ethynyl, propargyl (2-propynyl), 1-propynyl, but-1-ynyl, but-2-ynyl.

Cycloalkyl includes polycyclic saturated hydrocarbon radicals having up to 14 carbon atoms, namely monocyclic C₃-C₁₂-, preferably C₃-C₈-alkyl, in particular C₃-C₆-alkyl such as, for example, cyclopropyl, cyclobutyl, cyclopentyl, cyclohexyl, cycloheptyl, cyclooctyl, cyclononyl, and polycyclic alkyl, i.e, preferably bicyclic and tricyclic, optionally spirocyclic C₇-C₁₄-alkyl, such as, for example, bicyclo[2.2.1]-hept-1-yl, bicyclo[2.2.1]-hept-2-yl, bicyclo[2.2.1]-hept-7-yl, bicyclo[2.2.2]-oct-2-yl, bicyclo[3.2.1]-oct-2-yl, bicyclo[3.2.2]-non-2-yl and adamantyl.

Aryl is for the purposes of the invention an aromatic radical preferably having 6 to 10 carbon atoms. Preferred aryl radicals are phenyl and naphthyl.

Alkoxy is for the purposes of the invention preferably a straight-chain or branched alkoxy radical in particular having 1 to 6, 1 to 4 or 1 to 3 carbon atoms. A straight-chain or branched alkoxy radical having 1 to 3 carbon atoms is preferred. Those which may be mentioned by way of example and preferably are: methoxy, ethoxy, n-propoxy, isopropoxy, t-butoxy, n-pentoxy and n-hexoxy.

Alkoxycarbonyl is for the purposes of the invention preferably a straight-chain or branched alkoxy radical having 1 to 6 or 1 to 4 carbon atoms, which is linked via a carbonyl group. A straight-chain or branched alkoxycarbonyl radical having 1 to 4 carbon atoms is preferred. Those which may be mentioned by way of example and preferably are: methoxycarbonyl, ethoxycarbonyl, n-propoxycarbonyl, isopropoxycarbonyl and t-butoxycarbonyl.

Monoalkylamino (alkylamino) is for the purposes of the invention an amino group having one straight-chain or branched alkyl substituent which preferably has 1 to 6, 1 to 4 or 1 or 2 carbon atoms. A straight-chain or branched monoalkylamino radical having 1 to 4 carbon atoms is preferred. Those which may be mentioned by way of example and preferably are: methylamino, ethylamino, n-propylamino, isopropylamino, t-butylamino, n-pentylamino and n-hexylamino.

Dialkylamino is for the purposes of the invention an amino group having two identical or different straight-chain or branched alkyl substituents, which preferably each have 1 to 6, 1 to 4 or 1 or 2 carbon atoms. Straight-chain or branched dialkylamino radicals having in each case 1, 2, 3 or 4 carbon atoms per alkyl substituent are preferred. Those which may be mentioned by way of example and preferably are: N,N-dimethylamino, N,N-diethylamino, N-ethyl-N-methylamino, N-methyl-N-n-propylamino, N-isopropyl-N-n-propylamino, N-t-butyl-N-methylamino, N-ethyl-N-n-pentylamino and N-n-hexyl-N-methylamino.

Monoalkylaminocarbonyl (alkylaminocarbonyl) or dialkylaminocarbonyl is for the purposes of the invention an amino group which is linked via a carbonyl group and which has one straight-chain or branched or two identical or different straight-chain or branched alkyl substituents each preferably having 1 to 4 or 1 or 2 carbon atoms. Those which may be mentioned by way of example and preferably are: methylaminocarbonyl, ethylaminocarbonyl, isopropylaminocarbonyl, t-butylaminocarbonyl, N,N-dimethylaminocarbonyl, N,N-diethylaminocarbonyl, N-ethyl-N-methylaminocarbonyl and N-t-butyl-N-methylaminocarbonyl.

Arylaminocarbonyl is for the purposes of the invention an aromatic radical having preferably 6 to 10 carbon atoms, which is linked via an aminocarbonyl group. Preferred radicals are phenylaminocarbonyl and naphthylaminocarbonyl.

Alkylcarbonylamino (acylamino) is for the purposes of the invention an amino group having a straight-chain or branched alkanoyl substituent which preferably has 1 to 6, 1 to 4 or 1 or 2 carbon atoms and is linked via the carbonyl group. A monoacylamino radical having 1 or 2 carbon atoms is preferred. Those which may be mentioned by way of example and preferably are: formamido, acetamido, propionamido, n-butyramido and pivaloylamido.

Alkoxycarbonylamino is for the purposes of the invention an amino group having a straight-chain or branched alkoxycarbonyl substituent which preferably has 1 to 6 or 1 to 4 carbon atoms in the alkoxy radical and is linked via the carbonyl group. An alkoxycarbonylamino radical having 1 to 4 carbon atoms is preferred. Those which may be mentioned by way of example and preferably are: methoxycarbonylamino, ethoxycarbonylamino, n-propoxycarbonylamino and t-butoxycarbonylamino.

Heterocyclyl (heterocycle) is a mono- or polycyclic, heterocyclic radical having 4 to 10 ring atoms and up to 3, preferably up to 1 heteroatoms or heterogroups from the series N, O, S, SO, SO₂. 4- to 8-membered, in particular 5- to 6-membered heterocyclyl is preferred. Mono- or bicyclic heterocyclyl is preferred. Monocyclic heterocyclyl is particularly preferred. N and O are preferred as heteroatoms. The heterocyclyl radicals may be saturated or partially unsaturated. Saturated heterocyclyl radicals are preferred. The heterocyclyl radicals may be linked via a carbon atom or a heteroatom. 5- to 6-membered, monocyclic saturated heterocyclyl radicals having up to two heteroatoms from the series O, N and S are particularly preferred. Those which may be mentioned by way of example and preferably are: oxetan-3-yl, pyrrolidin-2-yl, pyrrolidin-3-yl, pyrrolinyl, tetrahydrofuranyl, tetrahydrothienyl, pyranyl, piperidin-1-yl, piperidin-2-yl, piperidin-3-yl, piperidin-4-yl, thiopyranyl, morpholin-1-yl, morpholin-2-yl, morpholin-3-yl, perhydroazepinyl, piperazin-1-yl, piperazin-2-yl. A nitrogen heterocyclyl ring is in this connection a heterocycle which has only nitrogen atoms as heteroatoms.

Heteroaryl is an aromatic, mono- or bicyclic radical having 5 to 10 ring atoms and up to 5 heteroatoms from the series S, O and/or N. 5- to 6-membered heteroaryls having up to 4 heteroatoms are preferred. The heteroaryl radical may be linked via a carbon atom or heteroatom. Those which may be mentioned by way of example and preferably are: thienyl, furyl, pyrrolyl, thiazolyl, oxazolyl, imidazolyl, pyridyl, pyrimidyl, pyridazinyl, indolyl, indazolyl, benzofuranyl, benzothiophenyl, quinolinyl, isoquinolinyl.

Carbonyl is a —C(O) group. Correspondingly, arylcarbonyl, heterocyclylcarbonyl and heteroarylcarbonyl are substituted on the carbonyl group by the appropriate radicals, i.e. aryl, heterocyclyl etc.

Sulfonyl is an —S(O)₂ group. Correspondingly, alkylsulfonyl, arylsulfonyl, heterocyclylsulfonyl and heteroarylsulfonyl are substituted on the sulfonyl group by the appropriate radicals, i.e. alkyl, aryl etc.

Aminosulfonyl is an —S(O)₂NH₂ group. Correspondingly, alkylaminosulfonyl, dialkylaminosulfonyl, arylaminosulfonyl, heterocyclylaminosulfonyl and heteroarylaminosulfonyl are substituted on the amino group by the appropriate radicals, i.e. alkyl, aryl etc.

Halogen includes for the purposes of the invention fluorine, chlorine, bromine and iodine. Fluorine or chlorine are preferred.

The side group of an amino acid means for the purposes of the invention the organic radical of an α-amino acid molecule which is linked to the α-carbon atom of the amino acid. Preference is given in this connection to the residues of naturally occurring α-amino acids in the L or in the D configuration, especially naturally occurring α-amino acids in the natural L configuration.

These include for example hydrogen (glycine), methyl (alanine), prop-2-yl (valine), 2-methylprop-1-yl (leucine), 1-methylprop-1-yl (isoleucine), a (3-indolyl)methyl group (tryptophan), a benzyl group (phenylalanine), a methylthioethyl group (methionine), hydroxymethyl (serine), p-hydroxybenzyl (tyrosine), 1-hydroxyeth-1-yl (threonine), mercaptomethyl (cysteine), carbamoylmethyl (asparagine), carbamoylethyl (glutamine), carboxymethyl (aspartic acid), carboxyethyl (glutamic acid), 4-aminobut-1-yl (lysine), 3-guanidinoprop-1-yl (arginine), imidazol-4-ylmethyl (histidine), 3-ureidoprop-1-yl (citrulline), mercaptoethyl (homocysteine), hydroxyethyl (homoserine), 4-amino-3-hydroxybut-1-yl (hydroxylysine), 3-aminoprop-1-yl (ornithine), 2-hydroxy-3-aminoprop-1-yl (hydroxyornithine).

Carbonyl-linked amino acid residue is an amino acid residue which is linked via the carbonyl group of the amino acid acidic function. Preference is given in this connection to α-amino acids in the L or in the D configuration, especially naturally occurring α-amino acids in the natural L configuration, e.g. glycine, L-alanine and L-proline.

Amine-linked amino acid residue is an amino acid residue which is linked via the amino group of the amino acid. Preference is given in this connection to α-amino acids or β-amino acids. Particular preference is given in this connection to α-amino acids in the L or in the D configuration, especially naturally occurring α-amino acids in the natural L configuration, e.g. glycine (R⁵ is carboxylmethyl), alanine (R⁵ is 1-carboxyleth-1-yl). The acid function of the amino acid may also be in the form of an ester, e.g. methyl, ethyl, tert-butyl ester, or of an amide, e.g. aminocarbonyl, alkylaminocarbonyl, dialkylaminocarbonyl, benzylaminocarbonyl group.

Amino protective groups means for the purposes of the present invention those organic radicals with which amino groups can be protected temporarily from attack by reagents, so that reactions such as oxidation, reduction, substitution and condensation take place only at the desired (unprotected) sites. They are stable for the duration of the protection under all conditions of the reactions and purification operations to be carried out and can be eliminated again selectively and with high yield under mild conditions (Römpp Lexikon Chemie—Version 2.0, Stuttgart/New York: \acute{G}eorg Thieme Verlag 1999; T. W. Greene, P. G. Wuts, Protective Groups in Organic Synthesis, 3^{rd} ed., John Wiley, New York, 1999).

Preference is given in this connection to oxycarbonyl derivatives such as carbamates and especially the following groups: benzyloxycarbonyl, 4-bromobenzyloxycarbonyl, 2-chlorobenzyloxycarbonyl, 3-chlorobenzyloxycarbonyl, dichlorobenzyloxycarbonyl, 3,4-dimethoxybenzyloxycarbonyl, 3,5-dimethoxybenzyloxycarbonyl, 2,4-dimethoxybenzyloxycarbonyl, 4-methoxybenzyloxycarbonyl, 4-nitrobenzyloxycarbonyl, 2-nitrobenzyloxycarbonyl, 2-nitro-4,5-dimethoxybenzyloxycarbonyl, 3,4,5-trimethoxybenzyloxycarbonyl, methoxycarbonyl, ethoxycarbonyl, propoxycarbonyl, isopropoxycarbonyl, butoxycarbonyl, isobutoxycarbonyl, tert-butoxycarbonyl, pentoxycarbonyl, isopentoxycarbonyl, hexoxycarbonyl, cyclohexoxycarbonyl, octoxycarbonyl, 2-ethylhexoxycarbonyl, 2-iodohexoxycarbonyl, 2-bromoethoxycarbonyl, 2-chloroethoxycarbonyl, 2,2,2-trichloroethoxycarbonyl, 2,2,2-trichloro-tert-butoxycarbonyl, benzhydryloxycarbonyl, bis-(4-methoxyphenyl)methoxycarbonyl, phenacyloxycarbonyl, 2-trimethylsilylethoxycarbonyl, phenacyloxycarbonyl, 2-trimethylsilylethoxycarbonyl, 2-(di-n-butylmethylsilyl)ethoxycarbonyl, 2-triphenylsilylethoxycarbonyl, 2-(dimethyl-tert-butylsilyl)ethoxycarbonyl, methyloxycarbonyl, vinyloxycarbonyl, allyloxycarbonyl, phenoxycarbonyl, tolyloxycarbonyl, 2,4-dinitrophenoxycarbonyl, 4-nitrophenoxycarbonyl, 2,4,5-trichlorophenoxycarbonyl, naphthyloxycarboyl, fluorenyl-9-methoxycarbonyl, valeroyl, isovaleroyl, butyryl, ethylthiocarbonyl, methylthiocarbonyl, butylthiocarboyl, tert-butylthiocarbonyl, phenylthiocarbonyl, benzylthiocarbonyl, methylaminocarbonyl, ethylaminocarbonyl, propylaminocarbonyl, isopropylaminocarbonyl, formyl, acetyl, propionyl, pivaloyl, 2-chloroacetyl, 2-bromoacetyl, 2-iodoacetyl, 2,2,2-trifluoroacetyl, 2,2,2-trichloroacetyl, benzoyl, 4-chlorobenzoyl, 4-methoxybenzoyl, 4-nitrobenzyl, 4-nitrobenzoyl, naphthylcarbonyl, phenoxyacetyl, adamantylcarbonyl, dicyclohexylphosphoryl, diphenylphosphoryl, dibenzylphosphoryl, di-(4-nitrobenzyl)phosphoryl, phenoxyphenylphosphoryl, diethylphosphinyl, diphenylphosphinyl, phthaloyl, phthalimido or benzyloxymethylene.

Particular preference is given to tert-butyloxycarbonyl (Boc), 9-fluorenylmethyloxycarbonyl (FMOC), benzyloxycarbonyl (Cbz-/Z-) and allyloxycarbonyl (Aloc).

A symbol * on a bond denotes the point of linkage in the molecule.

Preference is given for the purposes of the present invention to compounds which correspond to the formula 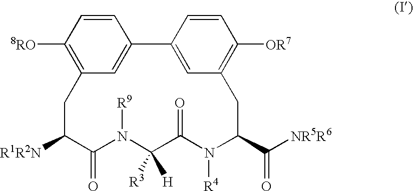
in which R¹ to R⁹ have the same meaning as in formula (I),
and the salts thereof, the solvates thereof and the solvates of the salts thereof.

Preference is given for the purposes of the present invention to compounds of the invention in which
R¹ is hydrogen, alkyl, aryl, heteroaryl, heterocyclyl, alkylcarbonyl, arylcarbonyl, heterocyclylcarbonyl, heteroarylcarbonyl, alkoxycarbonyl, aminocarbonyl, alkylaminocarbonyl, dialkylaminocarbonyl, alkylsulfonyl, arylsulfonyl, heterocyclylsulfonyl, heteroarylsulfonyl or a carbonyl-linked amino acid residue,
   where R¹ apart from hydrogen may be substituted by 0, 1, 2 or 3 substituents R¹⁻¹, where the substituents R¹⁻¹ are selected independently of one another from the group consisting of halogen, alkyl, trifluoromethyl, trifluoromethoxy, nitro, cyano, amino, alkylamino, dialkylamino, cycloalkyl, aryl, heteroaryl, heterocyclyl, hydroxy, alkoxy and carboxyl,
R² is hydrogen or alkyl,
   where R² apart from hydrogen may be substituted by 0, 1, 2 or 3 substituents R²⁻¹, where the substituents R²⁻¹ are selected independently of one another from the group consisting of halogen, amino, alkylamino and dialkylamino,
      or
R¹ and R² together with the nitrogen atom to which they are bonded form a heterocycle which may be substituted by 0, 1 or 2 substituents R¹⁻², where the substituents R¹⁻² are selected independently of one another from the group consisting of halogen, trifluoromethyl, amino, alkylamino, dialkylamino, cycloalkyl, aryl, heteroaryl, heterocyclyl, hydroxy, alkoxy, carboxyl, alkoxycarbonyl and aminocarbonyl,
R³ is hydrogen, alkyl or the side group of an amino acid, in which alkyl may be substituted by 0, 1, 2 or 3 substituents R³⁻¹, where the substituents R³⁻¹ are selected independently of one another from the group consisting of trifluoromethyl, nitro, amino, alkylamino, dialkylamino, cycloalkyl, aryl, heteroaryl, heterocyclyl, hydroxy, alkoxy, carboxyl, alkoxycarbonyl, aminocarbonyl, alkylaminocarbonyl and dialkylaminocarbonyl,
   in which cycloalkyl, aryl, heteroaryl and heterocyclyl may be substituted by 0, 1 or 2 substituents R³⁻², where the substituents R³⁻² are selected independently of one another from the group consisting of halogen, alkyl, trifluoromethyl and amino,
   and in which free amino groups in the side group of the amino acid may be substituted by alkyl, alkenyl, cycloalkyl, alkylcarbonyl, arylcarbonyl, heteroarylcarbonyl, heterocyclylcarbonyl, alkoxycarbonyl, aminocarbonyl, alkylaminocarbonyl, dialkylaminocarbonyl, arylaminocarbonyl, alkylsulfonyl, arylsulfonyl, heterocyclylsulfonyl or heteroarylsulfonyl,
R^{3′} is hydrogen or C₁-C₆-alkyl,
R⁴ is hydrogen, C₁-C₆-alkyl or C₃-C₈-cycloalkyl,
R⁵ is hydrogen, alkyl, alkenyl, cycloalkyl, aryl, heteroaryl, heterocyclyl or an amine-linked amino acid residue,
   where R⁵ may be substituted by 0, 1, 2 or 3 substituents R⁵⁻¹, where the substituents R⁵⁻¹ are selected independently of one another from the group consisting of halogen, alkyl, trifluoromethyl, trifluoromethoxy, cyano, amino, alkylamino, dialkylamino, cycloalkyl, aryl, heteroaryl, heterocyclyl, hydroxy, alkoxy, carboxyl, alkoxycarbonyl, aminocarbonyl, alkylaminocarbonyl and dialkylaminocarbonyl,
R⁶ is hydrogen, alkyl or cycloalkyl,
   or
R⁵ and R⁶ together with the nitrogen atom to which they are bonded form a heterocycle which may be substituted by 0, 1, 2 or 3 substituents R⁵⁻⁶, where the substituents R⁵⁻⁶ are selected independently of one another from the group consisting of halogen, alkyl, trifluoromethyl, nitro, amino, alkylamino, dialkylamino, cycloalkyl, aryl, halogenated aryl, heteroaryl, heterocyclyl, hydroxy, alkoxy, carboxyl, alkylcarbonyl, alkoxycarbonyl, aminocarbonyl, alkylaminocarbonyl and dialkylaminocarbonyl,
R⁷ is hydrogen or C₁-C₆-alkyl,
R⁸ is hydrogen or C₁-C₆-alkyl
   and
R⁹ is hydrogen or C₁-C₆-alkyl.

Preference is given for the purposes of the present invention also to compounds of the invention in which
R¹ is hydrogen, alkyl, alkylcarbonyl, arylcarbonyl, heterocyclylcarbonyl, heteroarylcarbonyl, alkoxycarbonyl or a carbonyl-linked amino acid residue,
   where R¹ apart from hydrogen may be substituted by 0, 1 or 2 substituents R¹⁻¹, where the substituents R¹⁻¹ are selected independently of one another from the group consisting of halogen, trifluoromethyl, amino, alkylamino, dialkylamino, phenyl, 5- to 6-membered heteroaryl, 5- to 6-membered heterocyclyl, hydroxy and alkoxy,
R² is hydrogen or methyl,
R³ is aminocarbonylmethyl, 3-aminopropyl, 2-hydroxy-3-aminopropyl, 3-guanidinopropyl, 2-aminocarbonylethyl, 2-hydroxycarbonylethyl, 4-aminobutyl, hydroxymethyl or 2-hydroxyethyl, 4-amino-3-hydroxybutan-1-yl,
   and in which free amino groups in the side group of the amino acid may be substituted by alkyl, alkenyl, C₃-C₆-cycloalkyl, alkylcarbonyl, phenylcarbonyl, 5- to 6-membered heteroarylcarbonyl, 5- to 6-membered heterocyclylcarbonyl, alkoxycarbonyl, aminocarbonyl, alkylaminocarbonyl, dialkylaminocarbonyl, phenylaminocarbonyl, alkylsulfonyl, arylsulfonyl, 5- to 6-membered heterocyclylsulfonyl or 5- to 6-membered heteroarylsulfonyl,
R^{3′} is hydrogen,
R⁴ is hydrogen or methyl,
R⁵ is hydrogen, alkyl, C₃-C₆-cycloalkyl, phenyl, 5- to 6-membered heteroaryl, 5- to 6-membered heterocyclyl or an amine-linked amino acid residue,
   where in the case where R⁵ is alkyl, C₃-C₆-cycloalkyl or 5- to 6-membered heterocyclyl, the latter may be substituted by 0, 1 or 2 substituents R⁵⁻², where the substituents R⁵⁻² are selected independently of one another from the group consisting of alkyl, trifluoromethyl, amino, alkylamino, dialkylamino, C₃-C₆-cycloalkyl, phenyl, 5- to 6-membered heteroaryl, 5- to 6-membered heterocyclyl, hydroxy, alkoxy, carboxyl, alkoxycarbonyl, aminocarbonyl, alkylaminocarbonyl and dialkylaminocarbonyl,
   and
   where in the case where R⁵ is phenyl or 5- to 6-membered heteroaryl, the latter may be substituted by 0, 1 or 2 substituents R⁵⁻³, where the substituents R⁵⁻³ are selected independently of one another from the group consisting of halogen, trifluoromethyl, trifluoromethoxy, amino, alkylamino, dialkylamino, C₃-C₆-cycloalkyl, 5- to 6-membered heteroaryl, 5- to 6-membered heterocyclyl, hydroxy, alkoxy, carboxyl, alkoxycarbonyl, aminocarbonyl, alkylaminocarbonyl and dialkylaminocarbonyl,
   and
   where in the case where R⁵ is amine-linked amino acid residue, the latter may be substituted by 0, 1 or 2 substituents R⁵⁻⁴, where the substituents R⁵⁻⁴ are selected independently of one another from the group consisting of halogen, trifluoromethyl, trifluoromethoxy, amino, alkylamino, dialkylamino, C₃-C₆-cycloalkyl, phenyl, 5- to 6-membered heteroaryl, 5- to 6-membered heterocyclyl, hydroxy, alkoxy, carboxyl, alkoxycarbonyl, aminocarbonyl, alkylaminocarbonyl and dialkylaminocarbonyl,
R⁶ is hydrogen, alkyl or C₃-C₆-cycloalkyl,
   or
R⁵ and R6 together with the nitrogen atom to which they are bonded form a 5- to 6-membered heterocycle which may be substituted by 0, 1 or 2 substituents R⁵⁻⁶, where the substituents R⁵⁻⁶ are selected independently of one another from the group consisting of amino, alkylamino, dialkylamino, C₃-C₆-cycloalkyl, phenyl, halogenated phenyl, 5- to 6-membered heteroaryl, hydroxy, alkoxy, carboxyl and aminocarbonyl,
R⁷ is hydrogen,
R⁸ is hydrogen,
   and
R⁹ is hydrogen or methyl.

Preference is given for the purposes of the present invention also to compounds of the invention in which
R¹ is hydrogen, alkyl or alkylcarbonyl,
R² is hydrogen,
R³ is alkyl or the side group of an amino acid, in which alkyl may be substituted by 0, 1, 2 or 3 substituents R³⁻¹, where the substituents R³⁻¹ are selected independently of one another from the group consisting of trifluoromethyl, nitro, amino, alkylamino, dialkylamino, cycloalkyl, aryl, heteroaryl, heterocyclyl, hydroxy, alkoxy, carboxyl, alkoxycarbonyl, aminocarbonyl, alkylaminocarbonyl, dialkylaminocarbonyl, guanidino and amidino,
   in which cycloalkyl, aryl, heteroaryl and heterocyclyl may be substituted by 0, 1 or 2 substituents R³⁻², where the substituents R³⁻² are selected independently of one another from the group consisting of halogen, alkyl, trifluoromethyl and amino,
   and in which free amino groups in the side group of the amino acid may be substituted by alkyl,
R^{3′} is hydrogen, C₁-C₆-alkyl or C₃-C₈-cycloalkyl,
R⁴ is hydrogen, C₁-C₆-alkyl or C₃-C₈-cycloalkyl,
R⁵ is hydrogen, alkyl, alkenyl, cycloalkyl, aryl, heteroaryl, heterocyclyl or an amine-linked amino acid residue,
   where alkyl, alkenyl, cycloalkyl, aryl, heteroaryl and heterocyclyl may be substituted by 0, 1, 2 or 3 substituents R⁵⁻¹, where the substituents R⁵⁻¹ are selected independently of one another from the group consisting of halogen, alkyl, trifluoromethyl, trifluoromethoxy, nitro, cyano, amino, alkylamino, dialkylamino, cycloalkyl, aryl, heteroaryl, heterocyclyl, hydroxy, alkoxy, carboxyl, alkoxycarbonyl, aminocarbonyl, alkylaminocarbonyl and dialkylaminocarbonyl,
      in which alkyl, alkylamino, dialkylamino, cycloalkyl, aryl, heteroaryl and heterocyclyl may be substituted by 0, 1, 2 or 3 substituents R⁵⁻², where the substituents R⁵⁻² are selected independently of one another from the group consisting of hydroxy, amino, carboxyl and aminocarbonyl,
R⁶ is hydrogen, alkyl or cycloalkyl,
   or
R⁵ and R⁶ together with the nitrogen atom to which they are bonded form a heterocycle which may be substituted by 0, 1, 2 or 3 substituents R⁵⁻⁶, where the substituents R⁵⁻⁶ are selected independently of one another from the group consisting of halogen, alkyl, amino, alkylamino, dialkylamino, hydroxy, alkoxy, carboxyl, alkoxycarbonyl, aminocarbonyl, alkylaminocarbonyl and dialkylaminocarbonyl,
R⁷ is hydrogen, C₁-C₆-alkyl, alkylcarbonyl or C₃-C₈-cycloalkyl,
R⁸ is hydrogen,
   and
R⁹ is hydrogen.

Preference is given for the purposes of the present invention also to compounds of the invention in which
R¹ is hydrogen,
R² is hydrogen,
R³ is alkyl or the side group of an amino acid, in which alkyl may be substituted by 0, 1, 2 or 3 substituents R³⁻¹, where the substituents R³⁻¹ are selected independently of one another from the group consisting of amino, alkylamino, dialkylamino, cycloalkyl, heteroaryl, heterocyclyl, hydroxy, alkoxy, carboxyl, alkoxycarbonyl, aminocarbonyl, alkylaminocarbonyl, dialkylaminocarbonyl, guanidino and amidino,
   in which cycloalkyl, heteroaryl and heterocyclyl may be substituted by 0, 1 or 2 substituents R³⁻², where the substituents R³⁻² are selected independently of one another from the group consisting of alkyl and amino,
R^{3′} is hydrogen,
R⁴ is hydrogen, C₁-C₆-alkyl or C₃-C₈-cycloalkyl,
R⁵ is hydrogen, alkyl, cycloalkyl, aryl, heteroaryl, heterocyclyl or an amine-linked amino acid residue,
   where alkyl, cycloalkyl, aryl, heteroaryl and heterocyclyl may be substituted by 0, 1, 2 or 3 substituents R⁵⁻¹, where the substituents R⁵⁻¹ are selected independently of one another from the group consisting of halogen, alkyl, trifluoromethyl, trifluoromethoxy, nitro, cyano, amino, alkylamino, dialkylamino, cycloalkyl, aryl, heteroaryl, heterocyclyl, hydroxy, alkoxy, carboxyl, alkoxycarbonyl, aminocarbonyl, alkylaminocarbonyl and dialkylaminocarbonyl,
      in which alkyl, alkylamino, dialkylamino, cycloalkyl, aryl, heteroaryl and heterocyclyl may be substituted by 0, 1, 2 or 3 substituents R⁵⁻², where the substituents R⁵⁻² are selected independently of one another from the group consisting of hydroxy, amino, carboxyl and aminocarbonyl,
R⁶ is hydrogen, alkyl or C₃-C₈-cycloalkyl,
   or
R⁵ and R⁶ together with the nitrogen atom to which they are bonded form a piperidinyl, morpholinyl, piperazinyl or pyrrolidinyl, where piperidinyl, morpholinyl, piperazinyl and pyrrolidinyl may be substituted by 0, 1, 2 or 3 substituents, where the substituents are selected independently of one another from the group consisting of alkyl, amino, alkylamino, dialkylamino, hydroxy, alkoxy, carboxyl, alkoxycarbonyl and aminocarbonyl,
R⁷ is hydrogen,
R⁸ is hydrogen,
   and
R⁹ is hydrogen.

Preference is given for the purposes of the present invention also to compounds of the invention in which
R¹ is hydrogen,
R² is hydrogen,
R³ is aminocarbonylmethyl, 3-aminoprop-1-yl, 2-hydroxy-3-aminoprop-1-yl, 1-hydroxy-3-aminoprop-1-yl, 3-guanidinoprop-1-yl, 2-aminocarbonylethyl, 2-hydroxycarbonylethyl, 4-aminobut-1-yl, hydroxymethyl, 2-hydroxyethyl, 2-aminoethyl, 4-amino-3-hydroxybut-1-yl or (1-piperidin-3-yl)methyl,
R^{3′} is hydrogen,
R⁴ is hydrogen, methyl, ethyl, isopropyl or cyclopropyl,
R⁵ is hydrogen, C₁-C₆-alkyl or C₃-C₈-cycloalkyl,
   where alkyl and cycloalkyl may be substituted by 0, 1, 2 or 3 substituents R⁵⁻¹, where the substituents R⁵⁻¹ are selected independently of one another from the group consisting of halogen, C₁-C₆-alkyl, trifluoromethyl, trifluoromethoxy, amino, C₁-C₆-alkylamino, C₁-C₆-dialkylamino, C₃-C₈-cycloalkyl, C₆-C₁₀-aryl, 5- to 10-membered heteroaryl, 5- to 7-membered heterocyclyl, hydroxy, alkoxy, carboxyl, C₁-C₆-alkoxycarbonyl, aminocarbonyl, C₁-C₆-alkylaminocarbonyl and C₁-C₆-dialkylaminocarbonyl,
R⁶ is hydrogen or methyl,
   or
R⁵ and R⁶ together with the nitrogen atom to which they are bonded form a piperidinyl or morpholinyl,
R⁷ is hydrogen,
R⁸ is hydrogen,
   and
R⁹ is hydrogen.

Particular preference is given for the purposes of the present invention to compounds of the invention in which
R¹ is hydrogen,
R² is hydrogen,
R³ is 3-aminoprop-1-yl or 2-hydroxy-3-aminoprop-1-yl,
R^{3′} is hydrogen,
R⁴ is hydrogen or methyl,
R⁵ is hydrogen, C₁-C₆-alkyl or cyclopropyl,
   where alkyl may be substituted by 0, 1, 2 or 3 substituents R⁵⁻¹, where the substituents R⁵⁻¹ are selected independently of one another from the group consisting of trifluoromethyl, amino, hydroxy, carboxyl, aminocarbonyl and phenyl,
R⁶ is hydrogen or methyl,
R⁷ is hydrogen,
R⁸ is hydrogen
   and
R⁹ is hydrogen.

Preference is given for the purposes of the present invention also to compounds of the invention in which R¹ is hydrogen.

Preference is given for the purposes of the present invention also to compounds of the invention in which R² is hydrogen.

Preference is given for the purposes of the present invention also to compounds of the invention in which R³ is 3-aminoprop-1-yl or 2-hydroxy-3-aminoprop-1-yl.

Preference is given for the purposes of the present invention also to compounds of the invention in which R^{3′} is hydrogen.

Preference is given for the purposes of the present invention also to compounds of the invention in which R⁴ is hydrogen or methyl.

Preference is given for the purposes of the present invention also to compounds of the invention in which
R⁵ is hydrogen, C₁-C₆-alkyl or cyclopropyl,
   where alkyl may be substituted by 0, 1, 2 or 3 substituents R⁵⁻¹, where the substituents R⁵⁻¹ are selected independently of one another from the group consisting of trifluoromethyl, amino, hydroxy, carboxyl, aminocarbonyl and phenyl.

Preference is given for the purposes of the present invention also to compounds of the invention in which R⁶ is hydrogen or methyl.

Preference is given for the purposes of the present invention also to compounds of the invention in which R⁵ and R⁶ together with the nitrogen atom to which they are bonded form a piperidinyl or morpholinyl.

Preference is given for the purposes of the present invention also to compounds of the invention in which R⁷ is hydrogen.

Preference is given for the purposes of the present invention also to compounds of the invention in which R⁸ is hydrogen.

Preference is given for the purposes of the present invention also to compounds of the invention in which R⁹ is hydrogen.

The invention further relates to a process for preparing the compounds of the formula (I), where the compounds of the formula 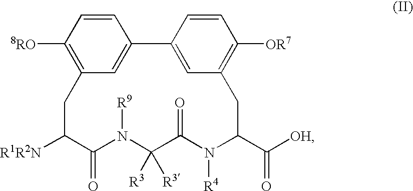
in which R¹ to R⁴ and R⁷ to R⁹ have the meaning indicated above, where the compounds (II) may where appropriate be in activated form (acyl donor),
are reacted with compounds of the formula
H—NR⁵R⁶ (III),
in which R⁵ and R⁶ have the meaning indicated above.

Where appropriate, reaction of compounds of the formula (II) with compounds of the formula (III) is preceded by blocking of reactive functionalities (e.g. free amino functions) in compounds of the formula (II). This takes place by standard methods of protective group chemistry. Preference is given to acid-labile protective groups on R¹ (or R²), or as substituents in the radicals R³ and R^{3′}, with particular preference for Boc. Reactive functionalities in the radicals R⁵ and R⁶ of compounds of the formula (III) are introduced already protected into the synthesis, with preference for acid-labile protective groups (e.g. Boc). After reaction has take place to give compounds of the formula (I), the protective groups can be eliminated by deprotection reaction. This takes place by standard methods of protective group chemistry. Deprotection reactions under acidic conditions are preferred.

If, for example, R² in compounds of the formula (I) is a protective group which can be selectively eliminated, deprotection (e.g. hydrogenolysis in the case of R²=Z) can be followed by functionalization of the exposed amino function (R²=H) with the desired substituent R².

Suitable for converting the compounds (II) into the activated form (acyl donor) are, for example, carbodiimides such as, for example, N,N′-diethyl-, N,N′,-dipropyl-, N,N′-diisopropyl-, N,N′-dicyclohexylcarbodiimide, N-(3-dimethylaminoisopropyl)-N′-ethylcarbodiimide hydrochloride (EDC) (where appropriate in the presence of pentafluorophenol (PFP)), N-cyclohexylcarbodiimide-N-propyloxymethyl-polystyrene (PS-carbodiimide) or carbonyl compounds such as carbonyldiimidazole, 1,2-oxazolium compounds such as 2-ethyl-5-phenyl-1,2-oxazolium 3-sulfate or 2-tert-butyl-5-methylisoxazolium perchlorate, or acylamino compounds such as 2-ethoxy-1-ethoxycarbonyl-1,2-dihydroquinoline, or propanephosphonic anhydride, or isobutyl chloroformate, or bis(2-oxo-3-oxazolidinyl)phosphoryl chloride or benzotriazolyloxytri(dimethylamino)phosphonium hexafluorophosphate or O-(benzotriazol-1-yl)-N,N,N′,N′-tetramethyluronium hexafluorophosphate (HBTU), 2-(2-oxo-1-(2H)-pyridyl)-1,1,3,3-tetramethyluronium tetrafluoroborate (TPTU) or O-(7-azabenzotriazol-1-yl)-N,N,N′,N′-tetramethyluronium hexafluorophosphate (HATU) or benzotriazol-1-yloxytris(dimethylamino)phosphonium hexafluorophosphate (BOP), or mixtures of these with bases, where appropriate in the presence of coupling additives such as 1-hydroxybenzotriazole (HOBt).

Examples of bases are alkali metal carbonates, such as, for example, sodium or potassium carbonate, or bicarbonate, or organic bases such as trialkylamines, e.g. triethylamine, N-methylmorpholine, N-methylpiperidine, 4-dimethylaminopyridine or diisopropylethylamine.

Solvents which are suitable in this case are inert organic solvents which are not changed under the reaction conditions. These include halohydrocarbons such as dichloromethane or trichloromethane, hydrocarbons such as benzene, toluene, tetrahydrofuran, dioxane, acetonitrile or dimethylformamide. It is likewise possible to employ mixtures of the solvents. Anhydrous dichloromethane and dimethylformamide are particularly preferred.

Activation with O-(7-azabenzotriazol-1-yl)-N,N,N′,N′-tetramethyluronium hexafluorophosphate (HATU) in dimethylformamide is preferred.

The compounds of the formula (III) are known or can be prepared in analogy to known processes.

The compounds of the formula (II) are known or can be prepared by cleaving the ester in compounds of the formula 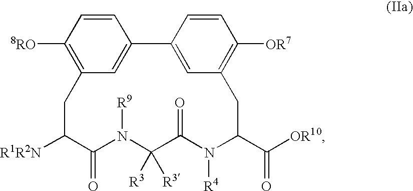
in which
R¹ to R⁴ and R⁷ to R⁹ have the meaning indicated above, and
R¹⁰ is benzyl (alternatively for alkyl, e.g. methyl or ethyl).

This ester cleavage takes place when R¹⁰ is benzyl preferably with hydrogen in the presence of palladium on carbon. Suitable solvents in this case are inert organic solvents which are not changed under the reaction conditions. These include halohydrocarbons such as dichloromethane or trichloromethane, hydrocarbon such as tetrahydrofuran, dioxane, dimethylformamide or alcohols (with preference for methanol, ethanol and isopropanol), where appropriate in the presence of acid with one or more acid equivalents. It is likewise possible to employ mixtures of the solvents. Formic acid in ethanol, aqueous acetic acid and THF are particularly preferred.

An alternative possibility is also to cleave the esters (R¹⁰=benzyl, alkyl) to the corresponding carboxylic acids by basic hydrolysis. Bases which are preferably employed are aqueous lithium or sodium hydroxide. Suitable solvents in this case are organic solvents which are partly or infinitely miscible with water. These include alcohols (with preference for methanol and ethanol), tetrahydrofuran, dioxane and dimethylformamide. It is likewise possible to employ mixtures of the solvents. Methanol, tetrahydrofuran and dimethylformamide are particularly preferred.

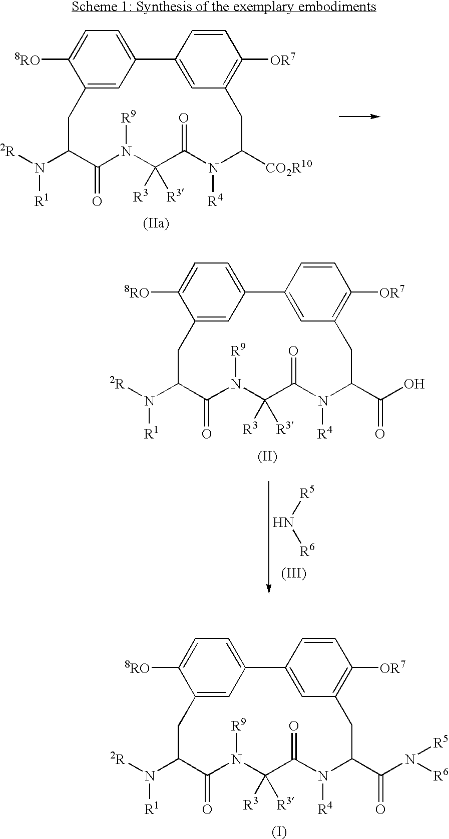

The compounds of the formula (IIa) can be prepared by cyclizing compounds of the formula 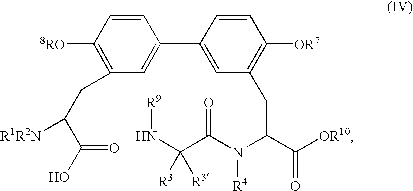
in which
R¹ to R⁴ and R⁷ to R¹⁰ have the meaning indicated above,

where these compounds are in activated form where appropriate, by peptide coupling. An alternative possibility is a multistage process in which compounds of the formula 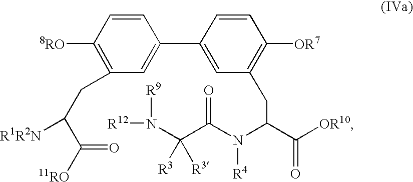
in which
R¹ to R⁴ and R⁷ to R¹⁰ have the meaning indicated above,
R¹¹ after activation is pentafluorophenol, and
R¹² is an amine protective group (preferably Boc),

are converted by protective group elimination of the amine protective group (to give R¹² equal to hydrogen) and subsequent cyclization under basic conditions into compounds of the formula (IIa).

Suitable for converting the compounds into the activated form are, for example, carbodiimides such as, for example, N,N′-diethyl-, N,N′,-dipropyl-, N,N′-diisopropyl-, N,N′-dicyclohexylcarbodiimide, N-(3-dimethylaminoisopropyl)-N′-ethylcarbodiimide hydrochloride (EDC) (where appropriate in the presence of pentafluorophenol (PFP)), N-cyclohexylcarbodiimide-N′-propyloxymethyl-polystyrene (PS-carbodiimide) or carbonyl compounds such as carbonyldiimidazole, 1,2-oxazolium compounds such as 2-ethyl-5-phenyl-1,2-oxazolium 3-sulfate or 2-tert-butyl-5-methylisoxazolium perchlorate, or acylamino compounds such as 2-ethoxy-1-ethoxycarbonyl-1,2-dihydroquinoline, or propanephosphonic anhydride, or isobutyl chloroformate, or bis(2-oxo-3-oxazolidinyl)phosphoryl chloride or benzotriazolyloxytri(dimethylamino)phosphonium hexafluorophosphate or O-(benzotriazol-1-yl)-N,N,N′,N′-tetramethyluronium hexafluorophosphate (HBTU), 2-(2-oxo-1-(2H)-pyridyl)-1,1,3,3-tetramethyluronium tetrafluoroborate (TPTU) or O-(7-azabenzotriazol-1-yl)-N,N,N′,N′-tetramethyluronium hexafluorophosphate (HATU) or benzotriazol-1-yloxytris(dimethylamino)phosphonium hexafluorophosphate (BOP), or mixtures of these with bases, where appropriate in the presence of 1-hydroxybenzotriazole (HOBt).

Examples of bases are alkali metal carbonates, such as, for example, sodium or potassium carbonate, or bicarbonate, or preferably organic bases such as trialkylamines, e.g. triethylamine, N-methylmorpholine, N-methylpiperidine, 4-dimethylaminopyridine or diisopropylethylamine.

Solvents which are suitable in this case are inert organic solvents which are not changed under the reaction conditions. These include halohydrocarbons such as dichloromethane or trichloromethane, hydrocarbons such as benzene, toluene, tetrahydrofuran, dioxane, dimethylformamide or acetonitrile. It is likewise possible to employ mixtures of the solvents. Dichloromethane and dimethylformamide are particularly preferred.

Activation in the form of a pentafluorophenyl ester (R¹¹=C₆F₅) and subsequent base-catalyzed ring closure is particularly preferred.

The compounds of the formula (IV) are known, can be prepared in analogy to known processes or by reacting compounds of the formula 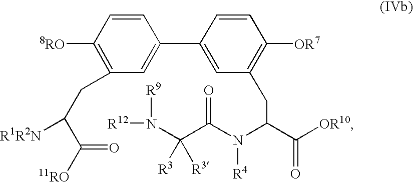
in which
R¹ to R⁴ and R⁷ to R¹⁰ and R¹² have the meaning indicated above, and
R¹¹ is a silyl protective group, in particular 2-(trimethylsilyl)ethyl,
   after elimination of the protective group on R¹², with fluoride, in particular with tetrabutylammonium fluoride.

The suitable solvents in this case are inert organic solvents which are not changed under the reaction conditions. These include halohydrocarbons such as dichloromethane, hydrocarbons such as benzene, toluene, tetrahydrofuran, dioxane and dimethylformamide. It is likewise possible to employ mixtures of the solvents. The preferred solvents are tetrahydrofuran and dimethylformamide.

The compounds of the formula (IVb) are known, can be prepared in analogy to known processes, or by reacting compounds of the formula 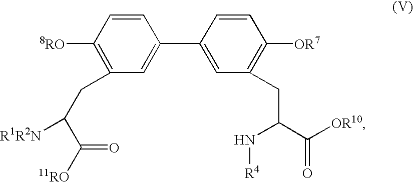
in which
R¹, R², R⁴, R⁷, R⁸ and R¹⁰ have the meaning indicated above,
R¹¹ is a silyl protective group,
   with compounds of the formula 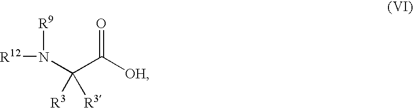
   in which
R³, R^{3′}, R⁹ and R¹² have the meaning indicated above, and
   where the compounds may where appropriate be in activated form.

Suitable for converting the compounds into the activated form are, for example, carbodiimides such as, for example, N,N′-diethyl-, N,N′,-dipropyl-, N,N′-diisopropyl-, N,N′-dicyclohexylcarbodiimide, N-(3-dimethylaminoisopropyl)-N′-ethylcarbodiimide hydrochloride (EDC) (where appropriate in the presence of pentafluorophenol (PFP)), N-cyclohexylcarbodiimide-N′-propyloxymethyl-polystyrene (PS-carbodiimide) or carbonyl compounds such as carbonyldiimidazole, 1,2-oxazolium compounds such as 2-ethyl-5-phenyl-1,2-oxazolium 3-sulfate or 2-tert-butyl-5-methylisoxazolium perchlorate, or acylamino compounds such as 2-ethoxy-1-ethoxycarbonyl-1,2-dihydroquinoline, or propanephosphonic anhydride, or isobutyl chloroformate, or bis(2-oxo-3-oxazolidinyl)phosphoryl chloride or benzotriazolyloxytri(dimethylamino)phosphonium hexafluorophosphate or O-(benzotriazol-1-yl)-N,N,N′,N′-tetramethyluronium hexafluorophosphate (HBTU), 2-(2-oxo-1-(2H)-pyridyl)-1,1,3,3-tetramethyluronium tetrafluoroborate (TPTU) or O-(7-azabenzotriazol-1-yl)-N,N,N′,N′-tetramethyluronium hexafluorophosphate (HATU) or benzotriazol-1-yloxytris(dimethylamino)phosphonium hexafluorophosphate (BOP), or mixtures of these with bases, where appropriate with addition of coupling additives such as 1-hydroxybenzotriazole (HOBt).

Examples of bases are alkali metal carbonates, such as, for example, sodium or potassium carbonate, or bicarbonate, or preferably organic bases such as trialkylamines, e.g. triethylamine, N-methylmorpholine, N-methylpiperidine, 4-dimethylaminopyridine or diisopropylethylamine.

Solvents which are suitable in this case are inert organic solvents which are not changed under the reaction conditions. These include halohydrocarbons such as dichloromethane or trichloromethane, hydrocarbons such as benzene, toluene, acetonitrile, tetrahydrofuran, dioxane or dimethylformamide. It is likewise possible to employ mixtures of the solvents. Anhydrous dichloromethane and dimethylformamide are particularly preferred.

Reaction in the presence of HATU and N,N-diisopropylethylamine is particularly preferred.

The compounds of the formula (VI) are known or can be prepared in analogy to known processes.

The compounds of the formula (V) and their salts (e.g. hydrochlorides) are known, can be prepared in analogy to known processes, or by preparing compounds of the formula 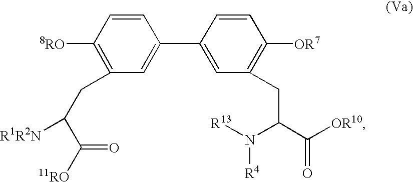
in which
R¹, R², R⁴, R⁷, R⁸ and R¹⁰ have the meaning indicated above,
R¹¹ is a silyl protective group, and
R¹³ is an amine protective group, in particular Boc, by deprotection on R¹³.

This takes place by standard methods of protective group chemistry, when R¹³ is Boc preferably with hydrogen chloride in dioxane.

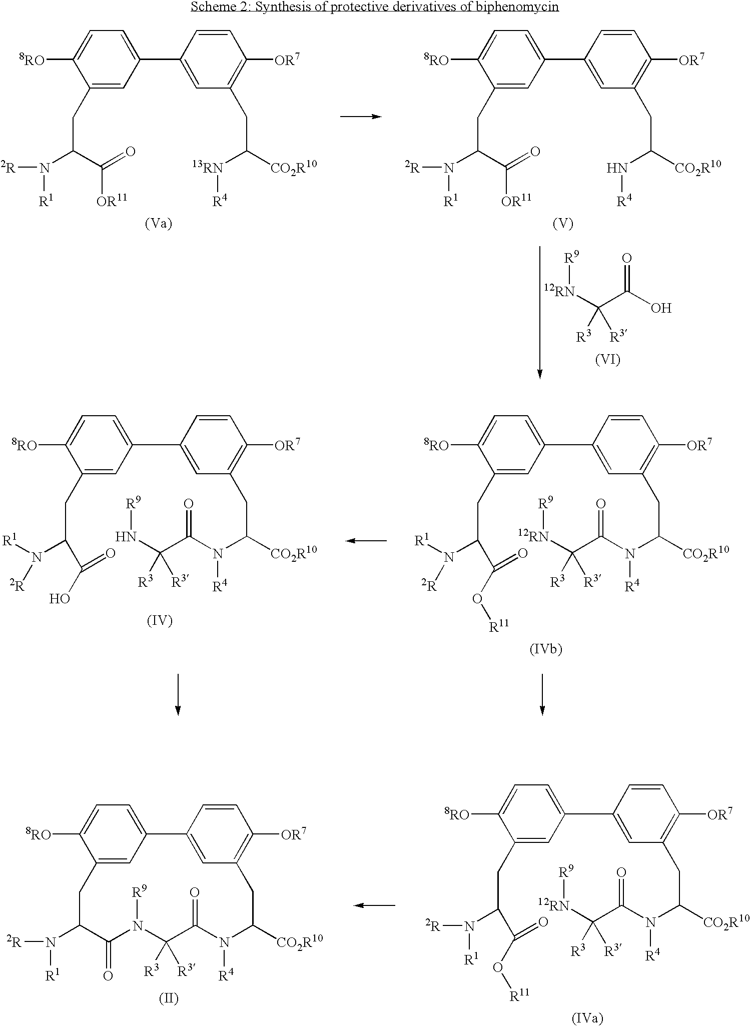

The compounds of the formula (Va) are known, can be prepared in analogy to known processes, or by reacting compounds of the formula 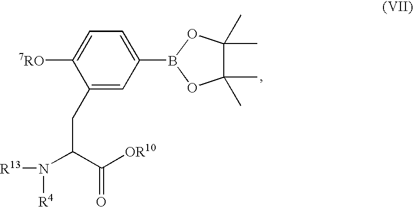
in which
R⁴ and R⁷ have the meaning indicated above,
R¹⁰ is benzyl or alkyl, and
R¹³ is an amino protective group (preferably Boc),
   with compounds of the formula 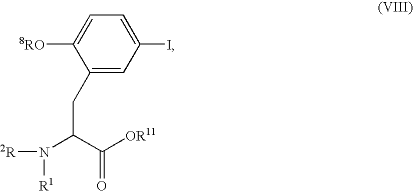
   in which
R¹, R² and R⁸ have the meaning indicated above, and
R¹¹ is a silyl protective group, in particular 2-(trimethylsilyl)ethyl.

The reaction, known as the Suzuki reaction (Synlett 1992, 207-210; Chem. Rev. 1995, 95, 2457-2483), takes place in the presence of palladium catalysts and a base, preferably in the presence of bis(diphenylphosphino)ferrocenepalladium(II) chloride and cesium carbonate.

Suitable solvents in this case are inert organic solvents which are not changed under the reaction conditions. These include hydrocarbons such as benzene, toluene, tetrahydrofuran, dioxane, dimethylformamide or dimethyl sulfoxide.

It is likewise possible to employ mixtures of the solvents. Dimethylformamide and dimethyl sulfoxide are particularly preferred.

The compounds of the formula (VII) are known, can be prepared in analogy to known processes, or by reacting compounds of the formula 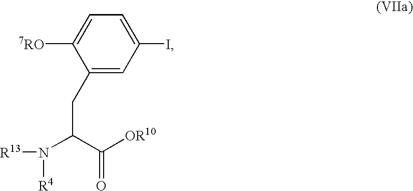
in which
R⁴ and R⁷ have the meaning indicated above,
R¹⁰ is benzyl or alkyl, and
R¹³ is an amino protective group (preferably Boc),
   with bis(pinacolato)diboron. This reaction, known as a special variant of the Suzuki reaction (J. Org. Chem. 1995, 7508-7510; Tetrahedron Lett., 1997, 3841-3844), takes place in the presence of palladium catalysts and a base, preferably in the presence of bis(diphenylphosphino)ferrocenepalladium(II) chloride and of potassium acetate.

Suitable solvents in this case are inert organic solvents which are not changed under the reaction conditions. These include hydrocarbons such as benzene, toluene, tetrahydrofuran, dioxane, dimethylformamide and dimethyl sulfoxide. It is likewise possible to employ mixtures of the solvents. Dimethylformamide and dimethyl sulfoxide are particularly preferred.

The compounds of the formula (VIIa) are known, can be prepared in analogy to known processes, or by reacting compounds of the formula 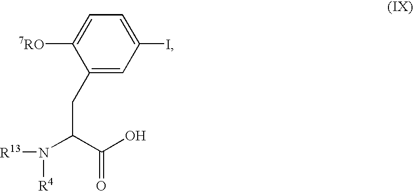
in which
R⁴ and R⁷ have the meaning indicated above, and
R¹³ is an amino protective group (preferably Boc),
   after activation of the free carboxylate function with ¹⁰R—OH (preferably benzyl alcohol, allyl alcohol and lower aliphatic alcohols) in the presence of 4-dimethylaminopyridine.

Suitable for converting the carboxylic acids into the activated form are, for example, carbodiimides such as, for example, N,N′-diethyl-, N,N′,-dipropyl-, N,N′-diisopropyl-, N,N′-dicyclobexylcarbodiimide, N-(3-dimethylaminoisopropyl)-N′-ethylcarbodiimide hydrochloride (EDC), N-cyclohexylcarbodiimide-N′-propyloxymethyl-polystyrene (PS-carbodiimide) or carbonyl compounds such as carbonyldiimidazole.

Suitable solvents in this case are inert organic solvents which are not changed under the reaction conditions. These include halohydrocarbons such as dichloromethane or trichloromethane, hydrocarbons such as benzene, toluene, acetonitrile, tetrahydrofuran, dioxane or dimethylformamide. It is likewise possible to employ mixtures of the solvents. Anhydrous dichloromethane and acetonitrile are particularly preferred.

Reactions with activation by EDC or DIC in absolute acetonitrile or dichloromethane at low temperature (−10° C.) are preferred.

The compounds of the formula (VIII) are known, can be prepared in analogy to known processes, or by reacting compounds of the formula 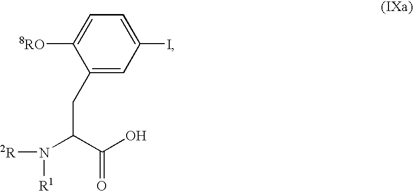
in which
R¹, R² and R⁸ have the meaning indicated above,
   after activation of the free carboxylate function with ¹¹R—OH (preferably 2-trimethylsilylethanol) in the presence of 4-dimethylaminopyridine.

Suitable for converting the carboxylic acids into the activated form are, for example, carbodiimides such as, for example, N,N′-diethyl-, N,N′,-dipropyl-, N,N′-diisopropyl-, N,N′-dicyclohexylcarbodiimide, N-(3-dimethylaminoisopropyl)-N′-ethylcarbodiimide hydrochloride (EDC), N-cyclohexylcarbodiimide-N′-propyloxymethyl-polystyrene (PS-carbodiimide) or carbonyl compounds such as carbonyldiimidazole.

Suitable solvents in this case are inert organic solvents which are not changed under the reaction conditions. These include halohydrocarbons such as dichloromethane or trichloromethane, hydrocarbons such as benzene, toluene, acetonitrile, tetrahydrofuran, dioxane or dimethylformamide. It is likewise possible to employ mixtures of the solvents. Anhydrous dichloromethane and acetonitrile are particularly preferred.

Reactions with activation by EDC or DIC in absolute acetonitrile or dichloromethane at low temperature (−10° C.) are preferred.

The carboxylic acids of the formula (IXa) are known, can be prepared in analogy to known processes, or by deprotecting compounds of the formula 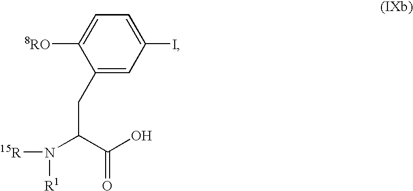
in which
R¹ and R⁸ have the meaning indicated above, and
R¹⁵ is an amino protective group, in particular Boc,
   in the first stage on R¹⁵. This takes place by standard methods of protective group chemistry, when R¹⁵ is Boc preferably with hydrogen chloride in dioxane or with trifluoroacetic acid in dichloromethane in the presence of small amounts of water.

The resulting free amine 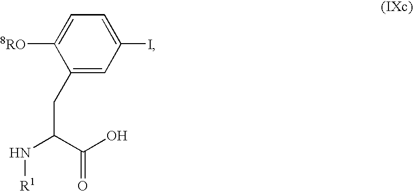
in which
R¹ and R⁸ have the meaning indicated above,
   where the amine may where appropriate be in the form of a salt, preferably hydrochloride or trifluoroacetate,
   is reacted in the second stage with R²—X, in which R² has the meaning indicated above, and X is a leaving group, in the presence of a base in inert solvents, where appropriate in the presence of potassium iodide, preferably in a temperature range from 0° C. via room temperature to reflux of the solvent under atmospheric pressure around. Mesylate, tosylate, succinate or halogen are preferred for X, with chlorine, bromine or iodine being preferred for halogen.

Examples of bases are alkali metal carbonates such as, for example, sodium or potassium carbonate, or bicarbonate, or organic bases such as trialkylamines, e.g. triethylamine, N-methylpiperidine, 4-dimethylaminopyridine or diisopropylethylamine.

Suitable solvents in this case are inert organic solvents which are not changed under the reaction conditions. These include halohydrocarbons such as dichloromethane or trichloromethane, hydrocarbons such as benzene, toluene, acetonitrile, tetrahydrofuran, dioxane, acetone or dimethylformamide. It is likewise possible to use mixtures of the solvents. Dimethylformamide and dichloromethane are particularly preferred. 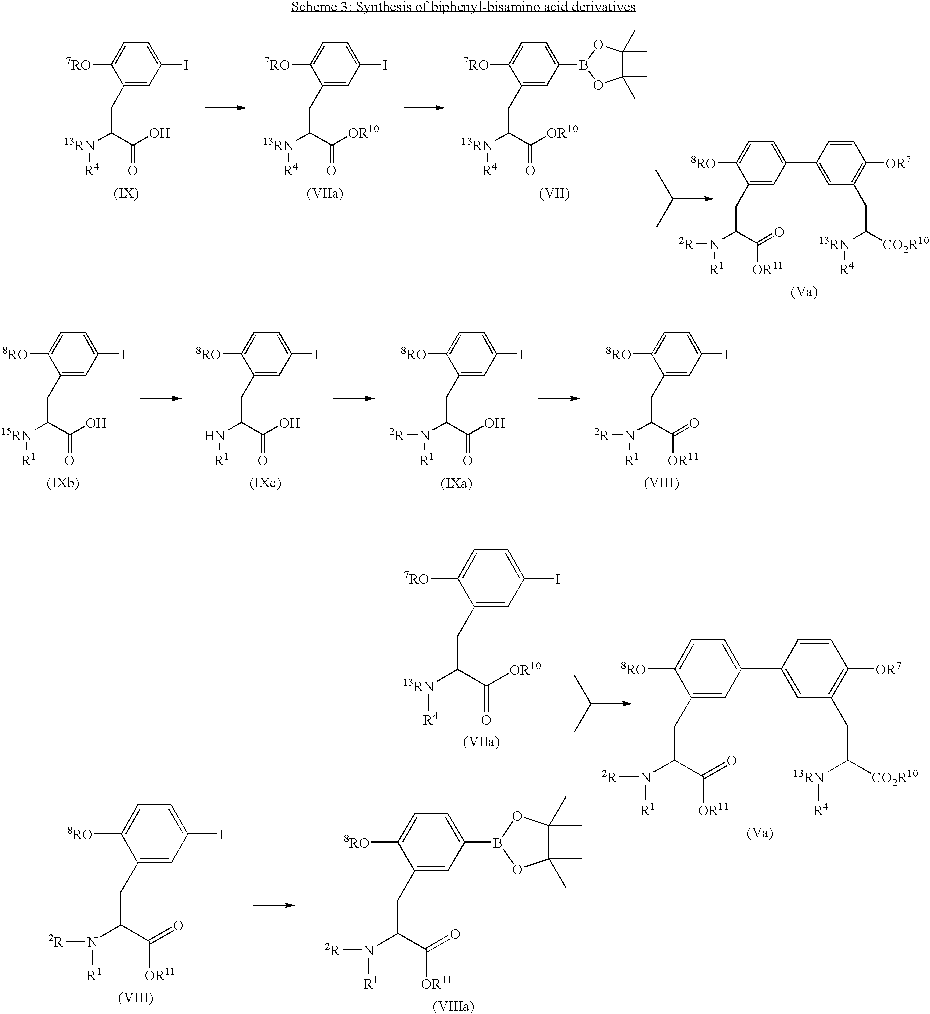
R² can optionally be a protective group (e.g. Z, i.e. benzyloxycarbonyl).

In an alternative process, the compounds of the formula (Va) can be prepared by reacting compounds of the formula 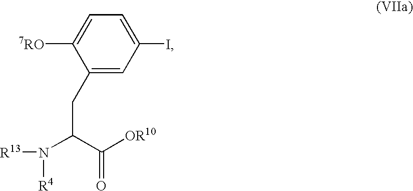
in which
R⁴ and R⁷ have the meaning indicated above,
R¹⁰ is benzyl or alkyl, and
R¹³ is an amino protective group (preferably Boc),
   with compounds of the formula 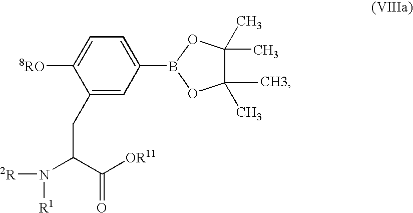
   in which
R¹, R² and R⁸ have the meaning indicated above, and
R¹¹ is a silyl protective group, in particular 2-(trimethylsilyl)ethyl.

The reaction, known as the Suzuki reaction (Synlett 1992, 207-210; Chem. Rev. 1995, 95, 2457-2483), takes place in the presence of palladium catalysts and a base, preferably in the presence of bis(diphenylphosphino)ferrocenepalladium(II) chloride and cesium carbonate.

Suitable solvents in this case are inert organic solvents which are not changed under the reaction conditions. These include hydrocarbons such as benzene, toluene, tetrahydrofuran, dioxane, dimethylformamide and dimethyl sulfoxide. It is likewise possible to employ mixtures of the solvents. Dimethylformamide and dimethyl sulfoxide are particularly preferred.

The compounds of the formula (VIIIa) can be prepared from the compounds of the formula (VIII) by the process described for compounds (VII).

The enantiomer pure compounds of the formulae (IX) and (IXb) are known or can be obtained from racemic precursors by known processes, such as, for example, crystallization with chiral amine bases or by chromatography on chiral stationary phases.

The compounds of the formulae (IX) and (IXb) are known, can be prepared in analogy to known processes, or by decarboxylating compounds of the formulae 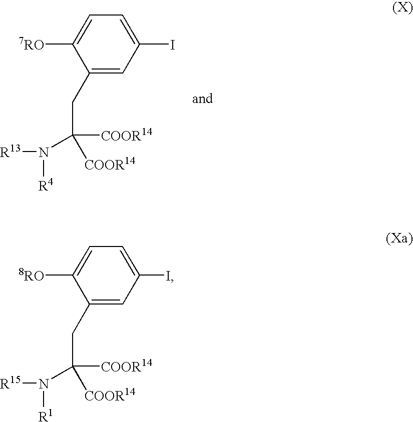
in which
R⁴ and R⁷ and R¹ and R⁸ have the meaning indicated above,
R¹³ and R¹⁵ are an amino protective group, and
R¹⁴ is alkyl (particularly preferably ethyl).

This reaction preferably takes place in basic medium in a water-ethanol mixture.

The compounds of the formulae (X) and (Xa) are known, can be prepared in analogy to known processes, or by reacting compounds of the formulae 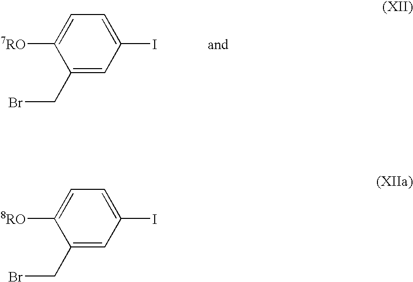
in which
R⁷ and R⁸ have the meaning indicated above,
   with compounds respectively of the formulae 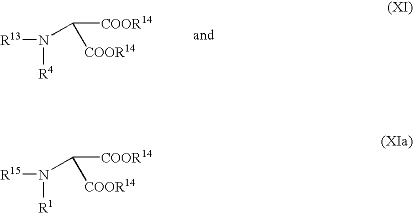
   in which
R⁴ and R¹ have the meaning indicated above,
R¹³ and R¹⁵ are an amino protective group, and
R¹⁴ is alkyl (preferably ethyl).

This reaction preferably takes place with alkali metal alcoholate in alcohol, in particular with sodium ethoxide in ethanol.

The compounds of the formulae (XII) and (XIIa) are known, can be prepared in analogy to known processes, or by reacting compounds of the formulae 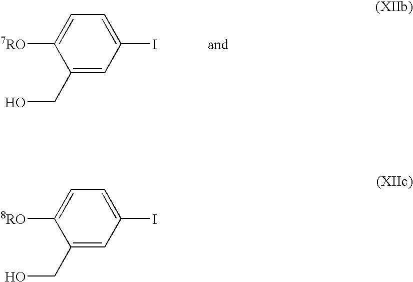
in which
R⁷ and R⁸ have the meaning indicated above,
   with phosphorus tribromide. The reaction preferably takes place in toluene.
   The compounds of the formulae (XIIb) and (XIIc) are known, can be prepared in analogy to known processes, or by reducing compounds of the formula 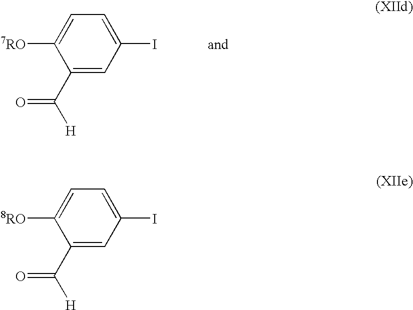
   in which
R⁷ and R⁸ have the meaning indicated above.

The reduction preferably takes place with diisobutylaluminum hydride solution in dichloromethane with subsequent addition of a saturated potassium sodium tartrate solution.

The compounds of the formulae (XIId) and (XIIe) are known, can be prepared in analogy to known processes, or by reacting 2-hydroxy-5-iodobenzaldehyde with compounds respectively of the formulae
R⁷—X and R⁸—X
(XIII) (XIIla),
   in which
R⁷ and R⁸ have the meaning indicated above, and
X is a leaving group, in inert solvents,
   where appropriate in the presence of a base, where appropriate in the presence of potassium iodide, preferably in a temperature range from room temperature to reflux of the solvent under atmospheric pressure. Mesylate, tosylate or halogen are preferred for X, with chlorine, bromine or iodine being preferred for halogen.

Examples of inert solvents are halohydrocarbons such as methylene chloride, trichloromethane or 1,2-dichloroethane, ethers such as dioxane, tetrahydrofuran or 1,2-dimethoxyethane, or other solvents such as acetone, dimethylformamide, dimethylacetamide, 2-butanone or acetonitrile, preferably tetrahydrofuran, methylene chloride, acetone, 2-butanone, acetonitrile, dimethylformamide or 1,2-dimethoxyethane. Dimethylformnamide is preferred.

Examples of bases are alkali metal carbonates such as cesium carbonate, sodium or potassium carbonate, or sodium or potassium methanolate, or sodium or potassium ethanolate or potassium tert-butoxide, or amides such as sodamide, lithiumbis(trimethylsilyl)amide or lithiumdiisopropylamide, or organometallic compounds such as butyllithium or phenyllithium, tertiary amine bases such as triethylamine or diisopropylethylamine, or other bases such as sodium hydride, DBU, preferably potassium tert-butoxide, cesium carbonate, DBU, sodium hydride, potassium carbonate or sodium carbonate. Potassium carbonate is preferred.

The compounds of the formulae (XIII) and (XIIIa) are known or can be prepared in analogy to known processes.

The preparation of the compounds of the invention can be illustrated by the following synthesis scheme. In this, to improve clarity, the roman numerals used in the description are retained but the scheme shows in some cases specific embodiments, in particular R¹⁴ in (XI) and (XIa) is ethyl and R¹³ and R¹⁵ is Boc.

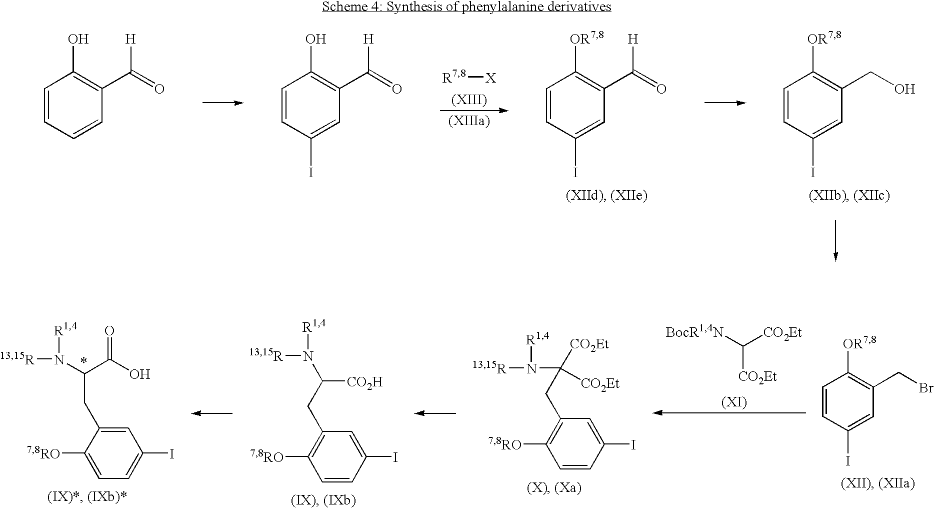

In an alternative process, the substituents R⁵ and R⁶ can also be introduced into the synthesis via the compounds of the formula (VII) or (VIIa). For this purpose, the acidic function of the compounds of the formula (VII) or (VIIa) is liberated under conditions known to the skilled worker and reacted with compounds of the formula (III) under conditions known to the skilled worker.

The compounds of the invention show an invaluable range of pharmacological and pharmacokinetic effects which could not have been predicted.

They are therefore suitable for use as medicaments for the treatment and/or prophylaxis of diseases in humans and animals.

The compounds of the invention can, because of their pharmacological properties, be employed alone or in combination with other active ingredients for the treatment and/or prevention of infectious diseases, in particular of bacterial infections.

It is possible for example to treat and/or prevent local and/or systemic diseases caused by the following pathogens or by mixtures of the following pathogens:

Gram-positive cocci, e.g. staphylococci (Staph. aureus, Staph. epidermidis) and streptococci (Strept. agalactiae, Strept. faecalis, Strept. pneumoniae, Strept. pyrogenes); gram-negative cocci (neisseria gonorrhoeae) and gram-negative rods such as enterobacteriaceae, e.g. Escherichia coli, Hemophilus influenzae, Citrobacter (Citrob. freundii, Citrob. divernis), Salmonella and Shigella; also klebsiellas (Klebs. pneumoniae, Klebs. oxytocy), Enterobacter (Ent. aerogenes, Ent. agglomerans), Hafnia, Serratia (Serr. marcescens), Proteus (Pr. mirabilis, Pr. rettgeri, Pr. vulgaris), Providencia, Yersinia, and the genus Acinetobacter. The antibacterial range also includes the genus Pseudomonas (Ps. aeruginosa, Ps. maltophilia) and strictly anaerobic bacteria such as, for example, Bacteroides fragilis, representatives of the genus Peptococcus, Peptostreptococcus, and the genus Clostridium; also mycoplasmas (M. pneumoniae, M. hominis, M. urealyticum) and mycobacteria, e.g. Mycobacterium tuberculosis.

The above list of pathogens is merely by way of example and is by no means to be interpreted restrictively. Examples which may be mentioned of diseases which may be caused by the pathogens or mixed infections mentioned and be prevented, improved or cured by the preparations of the invention which can be used topically are:

infectious diseases in humans, such as, for example, septic infections, bone and joint infections, skin infections, postoperative wound infections, abscesses, phlegmon, wound infections, infected bums, burn wounds, infections in the oral region, infections after dental operations, septic arthritis, mastitis, tonsillitis, genital infections and eye infections.

Apart from humans, bacterial infections can also be treated in other species. Examples which may be mentioned are:

pigs: coli diarrhea, enterotoxamia, sepsis, dysentery, salmonellosis, metritis-mastitis-agalactiae syndrome, mastitis;

ruminants (cattle, sheep, goats): diarrhea, sepsis, bronchopneumonia, salmonellosis, pasteurellosis, mycoplasmosis, genital infections;

horses: bronchopneumonias, joint ill, puerperal and postpuerperal infections, salmonellosis;

dogs and cats: bronchopneumonia, diarrhea, dermatitis, otitis, urinary tract infections, prostatitis;

poultry (chickens, turkeys, quail, pigeons, ornamental birds and others): mycoplasmosis, E. coli infections, chronic airway disorders, salmonellosis, pasteurellosis, psittacosis.

It is likewise possible to treat bacterial diseases in the rearing and management of productive and ornamental fish, in which case the antibacterial spectrum is extended beyond the pathogens mentioned above to further pathogens such as, for example, Pasteurella, Brucella, Campylobacter, Listeria, Erysipelothris, corynebacteria, Borellia, Treponema, Nocardia, Rikettsie, Yersinia.

The present invention additionally relates to compounds of the formula (I) for controlling diseases, especially bacterial diseases, to medicaments comprising compounds of the formula (I) and excipients, and to the use of compounds of the formula (I) for producing a medicament for the treatment of bacterial diseases.

The present invention further relates to medicaments which comprise at least one compound of the invention, preferably together with one or more pharmacologically acceptable excipients or carriers, and to the use thereof for the aforementioned purposes.

The active ingredient may act systemically and/or locally. For this purpose, it can be administered in a suitable manner such as, for example, by the oral, parenteral, pulmonary, nasal, sublingual, lingual, buccal, rectal, transdermal, conjunctival or otic route or as implant.

The active ingredient can be administered in administration forms suitable for these administration routes.

Suitable for oral administration are known administration forms which deliver the active ingredient rapidly and/or in a modified manner, such as, for example, tablets (uncoated and coated tablets, e.g. tablets provided with coatings resistant to gastric juice, or film-coated tablets), capsules, sugar-coated tablets, granules, pellets, powders, emulsions, suspensions, solutions and aerosols.

Parenteral administration can take place with avoidance of an absorption step (intravenous, intraarterial, intracardiac, intraspinal or intralumbal) or with inclusion of an absorption (intramuscular, subcutaneous, intracutaneous, percutaneous, or intraperitoneal). Administration forms suitable for parenteral administration are, inter alia, preparations for injection and infusion in the form of solutions, suspensions, emulsions, lyophilizates and sterile powders.

Suitable for the other administration routes are, for example, pharmaceutical forms for inhalation (inter alia powder inhalers, nebulizers), nasal drops/solutions, sprays; tablets or capsules for lingual, sublingual or buccal administration, suppositories, preparations for the ears and eyes, vaginal capsules, aqueous suspensions (lotions, shaking mixtures), lipophilic suspensions, ointments, creams, milk, pastes, dusting powders or implants.

The active ingredients can be converted in a manner known per se into the stated administration forms. This takes place with use of inert nontoxic, pharmaceutically suitable excipients. These include inter alia carriers (e.g. microcrystalline cellulose), solvents (e.g. liquid polyethylene glycols), emulsifiers (e.g. sodium dodecyl sulfate), dispersants (e.g. polyvinylpyrrolidone), synthetic and natural biopolymers (e.g. albumin), stabilizers (e.g. antioxidants such as ascorbic acid), colors (e.g. inorganic pigments such as iron oxides) or masking tastes and/or odors.

It has generally proved advantageous on parenteral administration to administer amounts of about 5 to 250 mg/kg of body weight every 24 h to achieve effective results. The amount on oral administration is about 5 to 100 mg/kg of body weight every 24 h.

It may nevertheless be necessary where appropriate to deviate from the stated amounts, in particular as a function of the body weight, administration route, individual behavior towards the active ingredient, nature of the preparation and time or interval over which administration takes place. Thus, it may be sufficient in some cases to make do with less than the aforementioned minimum amount, whereas in other cases the stated upper limit must be exceeded. Where larger amounts are administered, it may be advisable to divide these into a plurality of single doses over the day.

The percentage data in the following tests and examples are percentages by weight unless indicated otherwise; parts are parts by weight. Solvent ratios, dilution ratios and concentration data for liquid/liquid solutions are in each case based on volume.

### A. EXAMPLES

Abbreviations Used:

Aloc allyloxycarbonyl
aq. aqueous
Bn benzyl
Boc tert-butoxycarbonyl
CDCl₃ chloroform
CH cyclohexane
D dublet (in ¹H-NMR)
Dd dublet of dublets
DCM dichloromethane
DCC dicyclohexylcarbodiimide
DIC diisopropylcarbodiimide
DIPEA diisopropylethylamine
DMSO dimethyl sulfoxide
DMAP 4-N,N-dimethylaminopyridine
DMF dimethylformamide
EA ethyl acetate (acetic acid ethyl ester)
EDC N′-(3-dimethylaminopropyl)-N-ethylcarbodiimide×HCI
eq equivalent
ESI electrospray ionization (in MS)
HATU O-(7-azabenzotriazol-1-yl)-N,N,N′,N′-tetramethyluronium hexafluorophosphate
HBTU O-(benzotriazol-1-yl)-N,N,N′,N′-tetramethyluronium hexafluorophosphate
HOBt 1-hydroxy-1H-benzotriazole×H₂O
H hour(s)
HPLC high pressure, high performance liquid chromatography
LC-MS coupled liquid chromatography-mass spectroscopy
M multiplet (in ¹H-NMR)
Min minutes
MS mass spectroscopy
MeOH methanol
NMR nuclear magnetic resonance spectroscopy
MTBE methyl tert-butyl ether
Pd/C palladium/carbon
Q quartet (in ¹H-NMR)
R_{f} retention index (in TLC)
RT room temperature
Rₜ retention time (in HPLC)
S singlet (in ¹H-NMR)
sat. saturated
T triplet (in ¹H-NMR)
TBS tert-butyldimethylsilyl
THF tetrahydrofuran
TMSE 2-(trimethylsilyl)ethyl
TPTU 2-(2-oxo-1(2H)pyridyl)-1,1,3,3-tetramethyluronium tetrafluoroborate
Z benzyloxycarbonyl
   General LC-MS and HPLC Methods

Preparative RP-HPLC: column: YMC gel; eluent: acetonitrile/water (gradient); flow rate: 50 ml/min; temp.: 25° C.; detection UV 210 nm.

Method 1 (HPLC): column: Kromasil C18, L-R temperature: 30° C.; flow rate: 0.75 ml/min; eluent A: 0.01 M HClO₄, eluent B: acetonitrile, gradient: →0.5 min 98% A→4.5 min 10% A→6.5 min 10% A.

Method 2 (HPLC): column: Kromasil C18, 60*2 mm, L-R temperature: 30° C.; flow rate: 0.75 ml/min; eluent A: 0.01 M H₃PO₄, eluent B: acetonitrile, gradient: →0.5 min 90% A→4.5 min 10% A→6.5 min 10% A.

Method 3 (HPLC): column: Kromasil C18, 60*2 mm, L-R temperature: 30° C.; flow rate: 0.75 ml/min; eluent A: 0.005 M HClO₄, eluent B: acetonitrile, gradient: →0.5 min 98% A→4.5 min 10% A→6.5 min 10% A.

Method 4 (HPLC): column: symmetry C18 2.1×150 mm, column oven: 50° C.; flow rate: 0.6 ml/min; eluent A: 0.6 g of 30% hydrochloric acid/l of water, eluent B: acetonitrile, gradient: 0.0 min 90% A→4.0 min 10% A→9 min 10% A.

Method 5 (LC-MS): Instrument Micromass Quattro LCZ; column symmetry C18, 50 mm×2.1 mm, 3.5 μm; temperature: 40° C.; flow rate: 0.5 ml/min; eluent A: acetonitrile+0.1% formic acid, eluent B: water+0.1% formic acid, gradient: 0.0 min 10% A→4 min 90% A→6 min 90% A.

Method 6 (LC-MS): Instrument Micromass Platform LCZ; column symmetry C18, 50 mm×2.1 mm, 3.5 μm; temperature: 40° C.; flow rate: 0.5 ml/min; eluent A: acetonitrile+0.1% formic acid, eluent B: water+0.1% formic acid, gradient: 0.0 min 10% A→4 min 90% A→6 min 90% A.

Method 7 (LC-MS): Instrument Micromass Quattro LCZ; column symmetry C18, 50 mm×2.1 mm, 3.5 μm; temperature: 40° C.; flow rate: 0.5 ml/min; eluent A: acetonitrile+0.1% formic acid, eluent B: water+0.1% formic acid, gradient: 0.0 min 5% A→1 min 5% A→5 min 90% A→6 min 90% A.

Method 8 (HPLC): column: 250*4 mm, Kromasil 100, C-18, 5 μm; temperature: 40° C.; flow rate: 1 ml/min; eluent: acetonitrile 15% and 0.2% perchloric acid 85%; UV detection: 210 nm.

Method 9 (LC-MS): Instrument: Waters Alliance 2790 LC; column: symmetry C18, 50 mm×2.1 mm, 3.5 μm; eluent A: water+0.1% formic acid, eluent B: acetonitrile+0.1% formic acid, gradient: 0.0 min 5% B→5.0 min 10% B→6.0 min 10% B; temperature: 50° C.; flow rate: 1.0 ml/min; UV detection: 210 nm.

Method 10 (LC-MS): ZMD Waters; column: Inertsil ODS3 50 mm×2.1 mm, 3 μm; temperature: 40° C.; flow rate: 0.5 ml/min; eluent A: water+0.05% formic acid, eluent B: acetonitrile+0.05% formic acid, gradient: 0.0 min 5% B→12 min→100% B→15 min 100% B.

Method 11 (LC-MS): MAT 900, Finnigan MAT, Bremen; column: X-terra 50 mm×2.1 mm, 2.5 μm; temperature: 25° C.; flow rate: 0.5 ml/min; eluent A: water+0.01% formic acid, eluent B: acetonitrile+0.01% formic acid, gradient: 0.0 min 10% B→15 min→90% B→30 min 90% B.

Method 12 (LC-MS): TSQ 7000, Finnigan MAT, Bremen; column: Inertsil ODS3 50 mm×2.1 mm, 3 μm; temperature: 25° C.; flow rate: 0.5 ml/min; eluent A: water+0.05% formic acid, eluent B: acetonitrile+0.05% formic acid, gradient: 0.0 min 15% B→15 min→100% B→30 min 100% B.

Method 13 (LC-MS): 7 Tesla Apex II with external electrospray ion source, Bruker Daltronics; column: X-terra C18 50 mm×2.1 mm, 2.5 μm; temperature: 25° C.; flow rate: 0.5 ml/min; eluent A: water+0.1% formic acid, eluent B: acetonitrile+0.1% formic acid, gradient: 0.0 min 5% B→13 min→100% B→15 min 100% B.

Method 14 (HPLC): column: X-Terra™ from Waters, RP₈, 5 μm, 3.9×150 mm; start: 95% A, 5% B; 12 min: 5% A, 95% B. Eluent A: water+0.01% trifluoroacetic acid; eluent B: acetonitrile+0.01% trifluoroacetic acid; flow rate: 1.2 ml/min.

Method 15 (LC-MS): MS instrument type: Micromass ZQ; HPLC instrument type: Waters Alliance 2795; column: Merck Chromolith SpeedROD RP-18e 50×4.6 mm; eluent A: water+500 μl of 50% formic acid/l; eluent B: acetonitrile+500 μl of 50% formic acid/l; gradient: 0.0 min 10% B→3.0 min 95% B→4.0 min 95% B; oven: 35° C.; flow rate: 0.0 min 1.0 ml/min→3.0 min 3.0 ml/min→4.0 min 3.0 ml/min; UV detection: 210 nm.

Method 16 (LC-MS): MS instrument type: Micromass ZQ; HPLC instrument type: Waters Alliance 2795; column: Merck Chromolith SpeedROD RP-18e 50×4.6 mm; eluent A: water+500 μl of 50% formic acid/l; eluent B: acetonitrile+500 μl of 50% formic acid/l; gradient: 0.0 min 10% B→2.0 min 95% B→4.0 min 95% B; oven: 35° C.; flow rate: 0.0 min 1.0 ml/min→2.0 min 3.0 ml/min→4.0 min 3.0 m/min; UV detection: 210 nm.

Method 17 (LC-MS): Instrument: Micromass Platform LCZ with HPLC Agilent series 1100; column: Grom-SIL120 ODS-4 HE, 50 mm×2.0 mm, 3 μm; eluent A: 1 l of water+1 ml of 50% formic acid, eluent B: 1 l of acetonitrile+1 ml of 50% formic acid; gradient: 0.0 min 100% A→0.2 min 100% A→2.9 min 30% A→3.1 min 10% A→4.5 min 10% A; oven: 55° C.; flow rate: 0.8 ml/min; UV detection: 210 nm.

Method 18 (LC-MS): MS instrument type: Micromass ZQ; HPLC instrument type: Waters Alliance 2795; column: Merck Chromolith SpeedROD RP-18e 50×4.6 mm; eluent A: water+500 μl of 50% formic acid/l; eluent B: acetonitrile+500 μl of 50% formic acid/l; gradient: 0.0 min 10% B→3.0 min 95% B→4.0 min 95% B; oven: 35° C.; flow rate: 0.0 min 1.0 ml/min→3.0 min 3.0 ml/min→4.0 min 3.0 ml/min; UV detection: 210 nm.

Method 19 (LC-MS): MS instrument type: Micromass ZQ; HPLC instrument type: Waters Alliance 2790; column: Grom-Sil 120 ODS-4 HE 50 mm×2 mm, 3.0 μm; eluent B: acetonitrile+0.05% formic acid, eluent A: water+0.05% formic acid; gradient: 0.0 min 5% B→2.0 min 40% B→4.5 min 90% B→5.5 min 90% B; oven: 45° C.; flow rate: 0.0 min 0.75 ml/min→4.5 min 0.75 ml/min→5.5 min 1.25 ml/min; UV detection: 210 nm.

Method 20 (LC-MS): MS instrument type: Micromass ZQ; HPLC instrument type: Waters Alliance 2790; column: Uptisphere C 18, 50 mm×2.0 mm, 3.0 μm; eluent B: acetonitrile+0.05% formic acid, eluent A: water+0.05% formic acid; gradient: 0.0 min 5% B→2.0 min 40% B→4.5 min 90% B→5.5 min 90% B; oven: 45° C.; flow rate: 0.0 min 0.75 ml/min→4.5 min 0.75 ml/min→5.5 min 1.25 ml/min; UV detection: 210 nm.

Method 21 (LC-MS): Instrument: Micromass Quattro LCZ with HPLC Agilent Series 1100; column: UPTISPHERE HDO, 50 mm×2.0 mm, 3 μm; eluent A: 1 l of water+1 ml of 50% formic acid, eluent B: 1 l of acetonitrile+1 ml of 50% formic acid; gradient: 0.0 min 100% A→0.2 min 100% A→2.9 min 30% A→3.1 min 10% A→4.5 min 10% A; oven: 55° C.; flow rate: 0.8 ml/min; UV detection: 208-400 nm.

Method 22 (LC-MS): MS instrument type: Micromass ZQ; HPLC instrument type: HP 1100 Series; UV DAD; column: Grom-Sil 120 ODS-4 HE 50×2 mm, 3.0 μm; eluent A: water+500 μl of 50% formic acid/l; eluent B: acetonitrile+500 μl of 50% formic acid/l; gradient: 0.0 min 0% B→2.9 min 70% B→3.1 min 90% B→4.5 min 90% B; oven: 50° C.; flow rate: 0.8 ml/min; UV detection: 210 nm.

Method 23 (LC-MS): MS instrument type: Micromass ZQ; HPLC instrument type: Waters Alliance 2795; column: Phenomenex Synergi 2μ Hydro-RP Mercury 20×4 mm; eluent A: 1 l of water+0.5 ml of 50% formic acid, eluent B: 1 l of acetonitrile+0.5 ml of 50% formic acid; gradient: 0.0 min 90% A (flow rate: 1 ml/min)→2.5 min 30% A (flow rate: 2 ml/min)→3.0 min 5% A (flow rate: 2 ml/min)→4.5 min 5% A (flow rate: 2 ml/min); oven: 50° C.; UV detection: 210 nm.

Method 24 (LC-MS): MS instrument type: Micromass ZQ; HPLC instrument type: HP 1100 Series; UV DAD; column: Phenomenex Synergi 2μ Hydro-RP Mercury 20×4 mm; eluent A: 1 l of water+0.5 ml of 50% formic acid, eluent B: 1 l of acetonitrile+0.5 ml of 50% formic acid; gradient: 0.0 min 90% A (flow rate: 1 ml/min)→2.5 min 30% A (flow rate: 2 ml/min)→3.0 min 5% A (flow rate: 2 ml/min)→4.5 min 5% A (flow rate: 2 ml/min); oven: 50° C.; UV detection: 210 nm.

Method 25 (LC-MS): MS instrument type: Micromass ZQ; HPLC instrument type: HP 1100 Series; UV DAD; column: Grom-Sil 120 ODS-4 HE 50×2 mm, 3.0 μm; eluent A: water+500 μl of 50% formic acid/l, eluent B: acetonitrile +500 μl of 50% formic acid/l; gradient: 0.0 min 70% B→4.5 min 90% B; oven: 50° C.; flow rate: 0.8 ml/min, UV detection: 210 nm.

Method 26 (LC-MS): Instrument: Micromass Quattro LCZ with HPLC Agilent Series 1100; column: Grom-SIL120 ODS-4 HE, 50 mm×2.0 mm, 3 μm; eluent A: 1 l of water+1 ml of 50% formic acid, eluent B: 1 l of acetonitrile+1 ml of 50% formic acid; gradient: 0.0 min 100% A→0.2 min 100% A→2.9 min 30% A→3.1 min 10% A→4.5 min 10% A; oven: 55° C.; flow rate: 0.8 ml/min; UV detection: 208-400 nm.

Chemical Synthesis of the Examples

Synthesis of the Starting Compounds:

Synthesis of substituted phenylalanine derivatives with (−)-3-(2-benzyloxy-5-iodophenyl)-2(S)-tert-butoxycarbonylaminopropionic acid [(−)-6A] as example 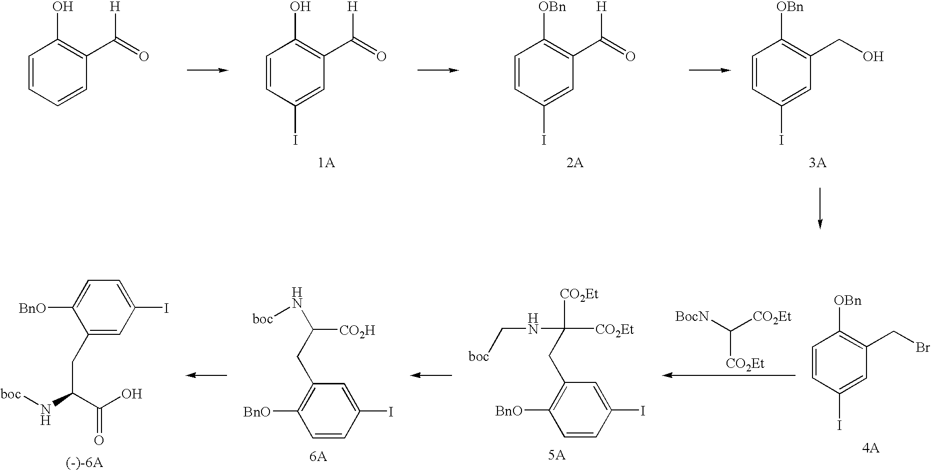
Synthesis of protected biphenyl-bisamino acids with 2(S)-trimethylsilanylethyl 2(S)-benzyloxycarbonylamino-3-[4,4′-bisbenzyloxy-3′-(2(S)benzyloxycarbonyl-2(S)-tert-butoxycarbonylaminoethyl)biphenyl-3-yl]propionate (12A) as example 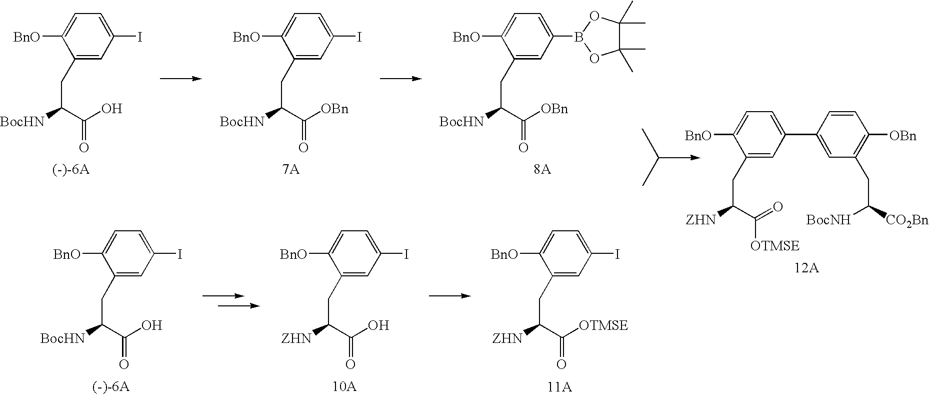
Synthesis of protected hydroxy ornithine derivatives with 5-benzyloxycarbonylamino-2(S)-tert-butoxycarbonylamino-4(R)-(tert-butyldimethylsilyloxy)pentanoic acid (14A) as example 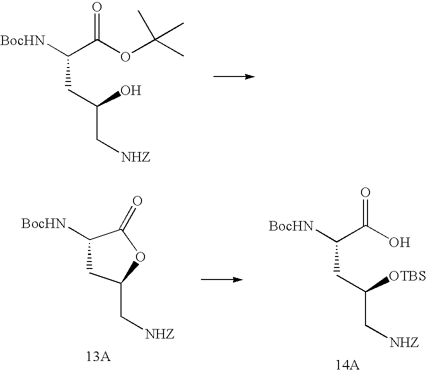
Synthesis of protected biphenomycin derivatives with (8S,11S,14S)-14-[(tert-butoxycarbonyl)amino]-11-{(2R)-3-[(tert-butoxycarbonyl)amino]-2-hydroxypropyl}-5,17-dihydroxy-10,13-dioxo-9,12-diazatricyclo[14.3.1.1^{2,6}]-henicosa-1(20),2(21),3,5,16,18-hexaene-8-carboxylic acid (21A) as example 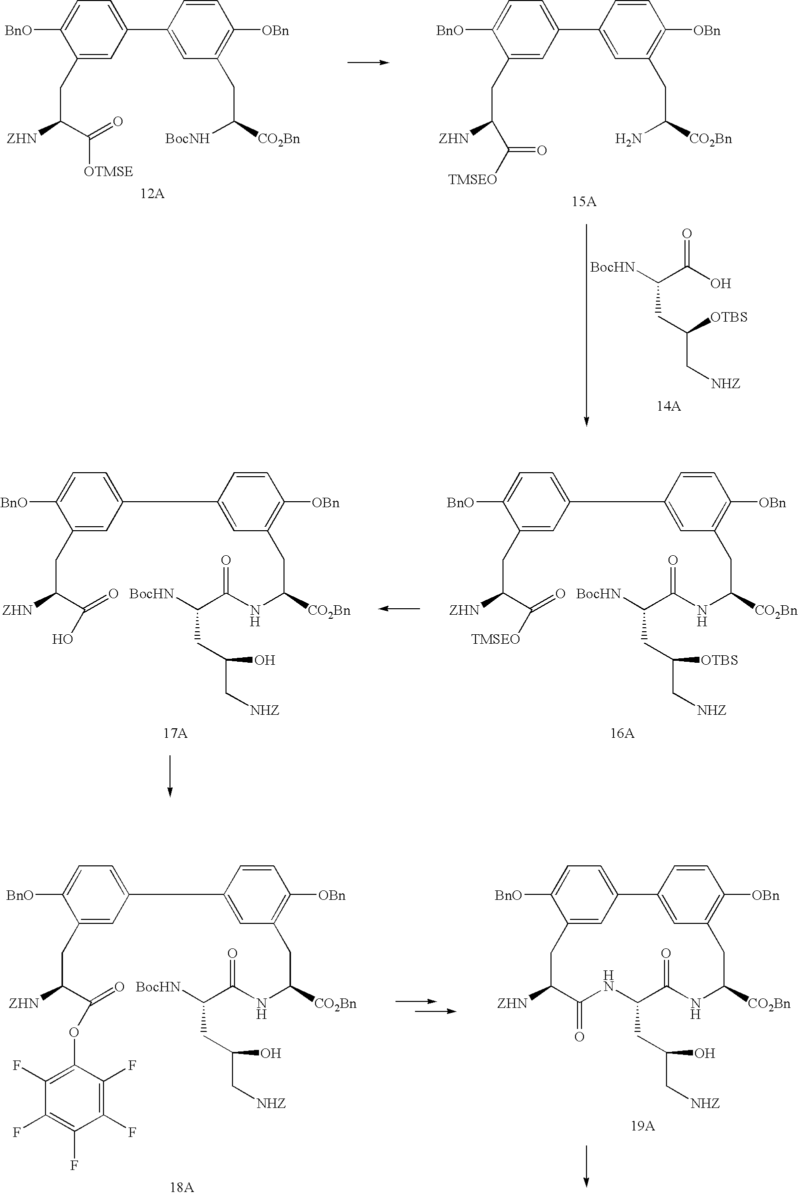 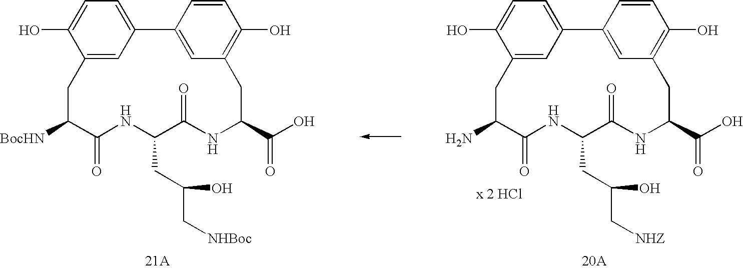
   Starting Compounds

### Example 1A

### 2-Hydroxy-5-iodobenzaldehyde

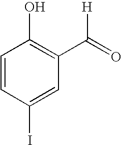

A solution of 250 g (1.54 mol) of iodine chloride in 600 ml of anhydrous dichloromethane is added dropwise over the course of 2 h to a solution of 188 g (1.54 mol) of salicylaldehyde in 1 l of anhydrous dichloromethane in a heat-dried flask under argon. After stirring at RT for 3 days, a saturated aqueous sodium sulfite solution is added with vigorous stirring. The organic phase is separated off, washed once with water and saturated aqueous sodium chloride solution and dried over sodium sulfate. The solvent is evaporated and the residue is recrystallized from ethyl acetate. 216 g (57% of theory) of the product are obtained.

LC-MS (ESI, Method 10): m/z=246 (M−H)⁻. ¹H-NMR (400 MHz, CDCl₃): δ=6.7 (d, 1H), 7.77 (dd, 1H), 7.85 (d, 1H), 9.83 (s, 1H), 10.95 (s, 1H).

### Example 2A

### 2-Benzyloxy-5-iodobenzaldehyde

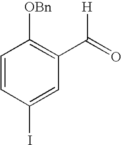

67.2 g (0.48 mol) of potassium carbonate are added to a solution of 100 g (0.40 mol) of 2-hydroxy-5-iodobenzaldehyde (Example 1A) in 1.5 l of dimethylformamide and, after a few minutes, 51 ml (0.44 mol) of benzyl chloride are added. The reaction mixture is stirred under reflux at 120° C. for 24 h. After stirring at RT for a further 24 h and addition of 1.5 l of water, a solid crystallizes out. The precipitate is filtered off with suction, washed twice with water and dried in vacuo. The solid is recrystallized from 230 ml of ethanol. 122.9 g (90% of theory) of the product are obtained.

LC-MS (ESI, Method 10): m/z=338 (M+H)⁺. ¹H-NMR (400 MHz, CDCl₃): δ=5.18 (s, 2H), 6.84 (d, 1H), 7.33-7.45 (m, 5H), 7.78 (dd, 1H), 8.12 (d, 1H), 10.4 (s, 1H).

### Example 3A

### (2-Benzyloxy-5-iodophenyl)methanol

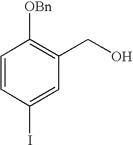

100 ml of 1 M diisobutylaluminum hydride solution in dichloromethane are added to a solution, cooled to 0° C., of 33.98 g (100.5 mmol) of 2-benzyloxy-5-iodobenzaldehyde (Example 2A) in 200 ml of dichloromethane. After stirring at 0° C. for 2 h, a saturated potassium sodium tartrate solution is added while cooling (highly exothermic reaction), and the reaction mixture is stirred for a further 2 h. After separation of the phases, the organic phase is washed twice with water and once with saturated aqueous sodium chloride solution and dried over sodium sulfate. The solvent is evaporated off in vacuo. 31.8 g (93% of theory) of the product are obtained.

1H-NMR (400 MHz, CDCl₃): δ=2.17 (t, 1H), 4.68 (d, 2H), 5.1 (s, 2H), 6.72 (d, 1H), 7.32-7.42 (m, 5H), 7.54 (dd, 1H), 7.63 (d, 1H).

### Example 4A

### 1-Benzyloxy-2-bromomethyl-4-iodobenzene

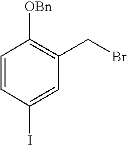

3.3 ml (35 mmol) of phosphorus tribromide are added dropwise to a solution of 35 g (103 mmol) of (2-benzyloxy-5-iodophenyl)methanol (Example 3A) in 350 ml of toluene at 40° C. The temperature of the reaction mixture is raised to 100° C. over the course of 15 min and is stirred at this temperature for a further IO min. After cooling the two phases are separated. The organic phase is washed twice with distilled water and once with saturated aqueous sodium chloride solution. The organic phase is dried over sodium sulfate and evaporated. The yield amounts to 41 g (99% of theory).

¹H-NMR (300 MHz, CDCl₃): δ=4.45 (s, 2H), 5.06 (s, 2H), 7.30 (m, 8H).

### Example 5A

### Diethyl 2-(2-benzyloxy-5-iodobenzyl)-2-tert-butoxycarbonylaminomalonate

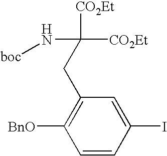

41 g (101.7 mmol) of 1-benzyloxy-2-bromomethyl-4-iodobenzene (Example 4A) are added to a solution of 28 g (101.7 mmol) of diethyl 2-[N-(tert-butoxycarbonyl)amino]malonate and 7.9 ml (101.7 mmol) of sodium ethoxide in 300 ml of ethanol. After stirring at RT for 3 h, the precipitated product is filtered off with suction. After drying in vacuo, 55 g (90% of theory) of product are isolated.

¹H-NMR (400 MHz, CDCl₃): δ=1.12 (t, 6H), 1.46 (s, 9H), 3.68 (s, 2H), 3.8-3.9 (m, 2H), 4.15-4.25 (m, 2H), 5.0 (s, 2H), 5.7 (s, 1H), 6.58 (d, 1H), 7.28-7.4 (m, 6H), 7.4 (dd, 1H).

### Example 6A

### (+/−)-3-(2-Benzyloxy-5-iodophenyl)-2-tert-butoxycarbonylaminopropionic acid

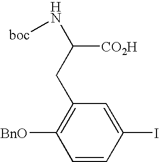

400 ml of 1 N sodium hydroxide solution are added to a suspension of 58 g (97 mmol) of diethyl 2-(2-benzyloxy-5-iodobenzyl)-2-tert-butoxycarbonylaminomalonate (Example 5A) in 800 ml of a mixture of ethanol and water (7:3). After 3 h under reflux and after cooling to room temperature, the pH of the reaction mixture is adjusted to about pH 2 with conc. hydrochloric acid. The reaction mixture is evaporated. The residue is taken up in MTBE and water. The aqueous phase is extracted three times with MTBE. The combined organic phases are dried over sodium sulfate, filtered and concentrated. Drying in vacuo results in 47 g (97% of theory) of the product.

¹H-NMR (400 MHz, DMSO): δ=1.32 (s, 9H), 2.68 (dd, 1H), 3.18 (dd, 1H), 4.25 (m, 1H), 5.15 (s, 2H), 6.88 (d, 1H), 7.08 (d, 1H), 7.30-7.40 (m, 3H), 7.45-7.55 (m, 3H).

### Example (−)-6A

### 3-(2-Benzyloxy-5-iodophenyl)-2(S)-tert-butoxycarbonylaminopropionic acid

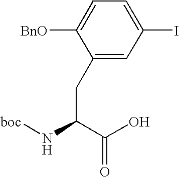

The racemate from Example 6A [(+/−)-3-(2-benzyloxy-5-iodophenyl)-2(S)-tert-butoxycarbonylaminopropionic acid] is separated on a chiral stationary silica gel phase based on the selector from poly(N-methacryloyl-L-leucine dicyclopropylmethylamide) using an i-hexane/ethyl acetate mixture as eluent. The enantiomer eluted first (98.9% ee) is dextrorotatory in dichloromethane ([α]²¹_{D}:+3.0°, c=0.54, dichloromethane) and corresponds to the (R) enantiomer Example (+)-6A, as was determined by single-crystal x-ray structural analysis. The purity of the second, levorotatory enantiomer Example (−)-6A, i.e. the (S) enantiomer, is >99% ee.

### Example 7A

### Benzyl 3-(2-benzyloxy-5-iodophenyl)-2(S)-tert-butoxycarbonylaminopropionate

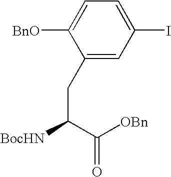

Under argon, 10 g (20.11 mmol) of (−)-3-(2-benzyloxy-5-iodophenyl)-2(S)-tert-butoxycarbonylaminopropionic acid [Example (−)-6A] are dissolved in 200 ml acetonitrile. To this are added 246 mg (2.01 mmol) of 4-dimethylaminopyridine and 4.16 ml (40.22 mmol) of benzyl alcohol. The mixture is cooled to −10°C., and 4.63 g (24.13 mmol) of EDC are added. The mixture is allowed slowly to reach RT and is stirred overnight. After about 16 h, the mixture is concentrated in vacuo, and the residue is purified by column chromatography on silica gel (mobile phase: dichloromethane). Yield: 10.65 g (88% of theory).

HPLC (Method 3): Rₜ=6.03 min; LC-MS (Method 9): Rₜ=4.70 min MS (DCI): m/z=605 (M+NH₄)⁺. ¹H-NMR (200 MHz, CDCl₃): δ=1.38 (s, 9H), 2.97 (dd, 1H), 3.12 (dd, 1H), 4.50-4.70 (m, 1H), 5.00-5.10 (m, 4H), 5.22 (d, 1H), 6.64 (d, 1H), 7.28-7.36 (m, 711), 7.37-7.52 (m, 5H).

### Example 8A

### Benzyl 3-[2-benzyloxy-5-(4,4,5,5-tetramethyl-[1,3,2]dioxaborolan-2-yl)phenyl]-2(S)-tert-butoxycarbonylaminopropionate

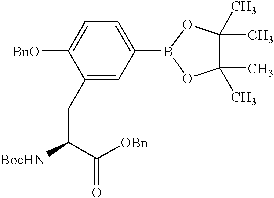

5.15 g (52.60 mmol) of potassium acetate are added to a solution of 10.30 g (17.53 mol) of benzyl 3-(2-benzyloxy-5-iodophenyl)-2(S)-tert-butoxycarbonylaminopropionate (Example 7A) in 70 ml of DMSO. The mixture is deoxygenated by passing argon through the vigorously stirred solution for 15 min. Then 5.17 g (20.16 mmol) of bis(pinacolato)diborane and 515 mg (0.70 mmol) of bis(diphenylphosphino)ferrocenepalladium(II) chloride are added. The mixture is then heated to 80° C. under a gentle stream of argon and after 6 h is cooled again. The mixture is purified by column chromatography on silica gel (mobile phase: dichloromethane). DMSO residues present are removed by Kugelrohr distillation. The residue is again purified by column chromatography on silica gel (mobile phase: cyclohexane:ethyl acetate 4:1).

Yield: 8.15 g (79% of theory). HLC (Method 3): Rₜ=6.26 min. LC-MS (Method 6): Rₜ=5.93 and 6.09 min. MS (EI): m/z=588 (M+H)⁺. ¹H-NMR (200 MHz, CDCl₃): δ=1.26 (s, 6H), 1.33 (s, 9H), 1.36 (s, 6H), 2.91-3.10 (m, 1H), 3.12-3.28 (m, 1H), 4.49-4.68 (m, 1H), 5.05 (dd, 2H), 5.11 (dd, 2H), 5.30 (d, 1H), 6.90 (d, 1H), 7.27-7.37 (m, 7H), 7.38-7.42 (m, 3H), 7.55-7.62 (m, 1H), 7.67 (dd, 1H).

### Example 9A

### 2(S)-Amino-3-(2-benzyloxy-5-iodophenyl)propionic acid hydrochloride

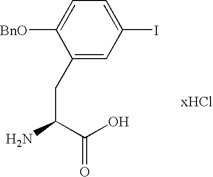

12 g (24.13 mmol) of 3-(2-benzyloxy-5-iodophenyl)-2(S)-tert-butoxycarbonylaminopropionic acid [Example (−)-6A] are put under argon into 60 ml of 4 M hydrochloric acid solution in dioxane and stirred at RT for 2 h. The reaction solution is concentrated and dried under high vacuum.

Yield: 10.47 g (100% of theory). HPLC (Method 3): Rₜ=4.10 min. MS (EI): m/z=398 (M+H—HCl)⁺. ¹H-NMR (200 MHz, CDCl₃): δ=3.17-3.31 (m, 1H), 3.33-3.47 (m, 1H), 4.22 (t, 1H), 5.13 (s, 2H), 6.69 (d, 1H), 7.24-7.40 (m, 2H), 7.41-7.45 (m, 2H), 7.48 (d, 1H), 7.52 (d, 1H), 7.60 (d, 1H), 8.66 (br,s, 2H).

### Example 10A

### 2(S)-Benzyloxycarbonylamino-3-(2-benzyloxy-5-iodophenyl)propionic acid

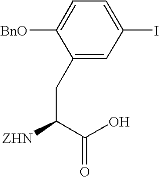

9.25 ml (53.09 mol) of N,N-diisopropylethylamine are added to a solution of 10.46 g (24.13 mmol) of 2(S)-amino-3-(2-benzyloxy-5-iodophenyl)propionic acid hydrochloride (Example 9A) in DMF. 6.615 g (26.54 mmol) of N-(benzyloxycarbonyl)succinimide (Z-OSuc) are added thereto. The resulting solution is stirred overnight and then evaporated in vacuo. The residue is taken up in dichloromethane and extracted twice each with 0.1 N hydrochloric acid solution and saturated aqueous sodium chloride solution. The organic phase is dried, filtered and concentrated. The mixture is purified by column chromatography on silica gel (mobile phase: cyclohexane/diethyl ether 9:1 to 8:2).

Yield: 8.30 g (65% of theory) HPLC (Method 3): Rₜ=5.01 min. MS (EI): m/z=532 (M+H)⁺. ¹H-NMR (200 MHz, DMSO): δ=3.14-3.3 (m, 2H), 4.25-4.45 (m, 1H), 4.97 (s, 2H), 5.14 (s, 2H), 6.88 (d, 1H), 7.20-7.56 (m, 12H), 7.62 (d, 1H), 12.73 (br,s, 1H).

### Example 11A

### (2-Trimethylsilyl)ethyl 2(S)-benzyloxycarbonylamino-3-(2-benzyloxy-5-iodophenyl)propionate

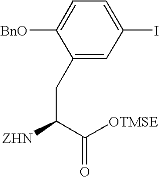

8.35 g (15.7 mmol) of 2(S)-benzyloxycarbonylamino-3-(2-benzyloxy-5-iodophenyl)propionic acid (Example 10A) are introduced into 150 ml of THF, and 2.14 g (18.07 mmol) of 2-trimethylsilylethanol and 250 mg (2.04 mmol) of 4-dimethylaminopyridine are added. The mixture is cooled to 0°, and 2.38 g (2.95 ml, 18.86 mmol) of N,N′-diisopropylcarbodiimide dissolved in 40 ml of THF are added. The mixture is stirred at RT overnight and evaporated in vacuo for working up. The residue is taken up in dichloromethane and extracted twice each with 0.1 N hydrochloric acid solution and saturated aqueous sodium chloride solution. The organic phase is dried, filtered and concentrated. The mixture is purified by column chromatography (silica gel, mobile phase: cyclohexane/diethyl ether 9:1 to 8:2).

Yield: 8.2 g (83% of theory). HPLC (Method 3): Rₜ=6.42 min MS (EI): m/z=532 (M+H)⁺. ¹H-NMR (300 MHz, CDCl₃): δ=0.01 (s, 9H), 0.88 (t, 2H), 2.96 (dd, 1H), 3.13 (dd, 1H), 4.04-4.17 (m, 2H), 4.51-4.62 (m. 1H), 4.95-5.05 (m, 4H), 5.44 (d, 1H), 6.64 (d, 1H), 7.25-7.33 (m, 7H), 7.37 (dd, 4H), 7.45 (dd, 1H).

### Example 12A

### 2-(Trimethylsilyl)ethyl 2(S)-benzyloxycarbonylamino-3-[4,4′-bisbenzyloxy-3′-(2(S)-benzyloxycarbonyl-2-tert-butoxycarbonylaminoethyl)biphenyl-3-yl]propionate

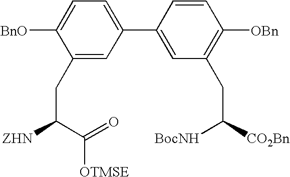 Method A:

45.8 mg (0.05 mmol) of bis(diphenylphosphino)ferrocenepalladium(II) chloride (PdCl₂(dppf)) and 0.325 g (1.0 mmol) of cesium carbonate are added to a solution of 0.316 g (0.5 mmol) of (2-trimethylsilyl)ethyl 2(S)-benzyloxycarbonylamino-3-(2-benzyloxy-5-iodophenyl)propionate (Example 11A) in 2.5 ml of degassed DMF under argon at RT. The reaction mixture is heated to 40° C. Over the course of 30 min, a solution of 0.294 g (0.5 mmol) of benzyl 3-[2-benzyloxy-5-(4,4,5,5-tetramethyl-[1,3,2]dioxaborolan-2-yl)phenyl]-2(S)-tert-butoxycarbonylaminopropionate (Example 8A) in 2.5 ml of degassed DMF is added dropwise. The reaction mixture is stirred at 40° C. for 4 h and at 50° C. for a further 2 h. The solvent is evaporated and the residue is taken up in ethyl acetate. The organic phase is extracted twice with water, dried over sodium sulfate and concentrated. The crude product is purified by chromatography on silica gel with dichloromethane/ethyl acetate (30/1). 0.320 g (66% of theory) of the product is obtained.

Method B:

A solution of 6.99 g (11.06 mmol) of (2-trimethylsilyl)ethyl 2(S)-benzyloxycarbonylamino-3-(2-benzyloxy-5-iodophenyl)propionate (Example 11A) and 6.50 g (11.06 mmol) of benzyl 3-[2-benzyloxy-5-(4,4,5,5-tetramethyl-[1,3,2]dioxaborolan-2-yl)phenyl]-2(S)-tert-butoxycarbonylaminopropionate (Example 8A) in 40 ml of DMF is degassed by passing argon through (about 30 min.). Then 812 mg (1.11 mmol) of bis(diphenylphosphino)ferrocenepalladium(II) chloride (PdCl₂(dppf)) and 7.21 g (22.13 mmol) of cesium carbonate are added thereto. A gentle stream of argon is passed over the reaction mixture, which is heated at 80° C. for 2.5 h. The mixture is cooled and purified by column chromatography on silica gel (mobile phase: cyclohexane/ethyl acetate 7:3). Before evaporation to dryness is complete, diisopropyl ether is added to the mixture. The resulting crystals are filtered off with suction and dried under high vacuum.

Yield: 6.54 g (61% of theory). HPLC (Method 3): Rₜ=7.65 min MS (EI): m/z=987 (M+Na), 965 (M+H)⁺. ¹H-NMR (200 MHz, CDCl₃): δ=0.00 (s, 9H), 0.90 (t, 2H), 1.37 (s, 9H), 3.02-3.35 (m, 4H) 4.06-4.25 (m, 2H), 4.55-4.73 (m, 2H), 4.98-5.18 (m, 8H), 5.40 (d, 1H), 5.63 (d, 1H), 6.88-7.00 (m, 2H), 7.19-7.39 (m, 20H), 7.42-7.53 (m, 4H).

### Example 13A

### N^{a}-(tert-Butoxycarbonyl)-N^{ε}(benzyloxycarbonyl)-(2S,4R)-hydroxyornithine lactone

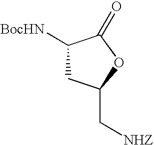

A solution of 7.60 g (17.3 mmol) of tert-butyl 5-benzyloxycarbonylamino-2(S)-tert-butoxycarbonylamino-4(R)-hydroxypentanoate (preparation described in Org. Lett. 2001, 3, 20, 3153-3155) in 516 ml of dichloromethane and 516 ml of trifluoroacetic acid is stirred at RT for 2 h. The solvent is evaporated. The remaining crude product is dissolved in 2.6 l of anhydrous methanol and, while stirring at 0° C., 6.3 g (28.8 mmol) of di-tert-butyl dicarbonate and 7.3 ml (52.43 mmol) of triethylamine are added. After 15 h, the reaction solution is evaporated and the residue is taken up in 1 l of ethyl acetate. After the phases have been separated, the organic phase is extracted twice with a 5% strength citric acid solution, twice with water and once with saturated aqueous sodium chloride solution, dried over sodium sulfate and concentrated. The crude product is purified by chromatography on silica gel with toluene/acetone (5/1). 4.92 g (78% of theory) of the product are obtained.

LC-HR-FT-ICR-MS (Method 13): calc. for C₁₈H₂₈N₃O₆ (M+NH₄)⁺382.19726 found 382.19703. ¹H-NMR (400 MHz, CDCl₃): δ=1.45 (s, 9H), 2.3-2.4 (mi, 1H), 2.45-2.55 (m, 1H), 3.3-3.4 (m, 1H), 3.5-3.6 (m, 1H), 4.17-4.28 (m, 1H), 4.7-4.8 (m, 1H), 5.0-5.15 (m, 4H), 7.3-7.4 (m, 5H).

### Example 14A

### 5-Benzyloxycarbonylamino-2(S)-tert-butoxycarbonylamino-4(R)-(tert-butyldimethylsilanyloxy)pentanoic acid

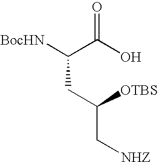 Method A:

2 ml of 1 M sodium hydroxide solution are added to a solution of 0.73 g (2 mmol) of N^{a}-(tert-butoxycarbonyl)-N^{ε(benzyloxycarbonyl)-(}2S,4R)-hydroxyornithine lactone (13A) in 50 ml of 1,4-dioxane at 0° C. The reaction solution is stirred for 2 h and then evaporated. The residue is taken up in 50 ml of dichloromethane. 1.12 ml (8 mmol) of triethylamine are added to this solution and, after a short time, 1.38 ml (6 mmol) of tert-butyldimethylsilyl trifluoromethanesulfonate are added dropwise. After stirring at RT for 3 h, the reaction mixture is diluted with dichloromethane. The organic phase is washed with 1 N sodium bicarbonate solution, dried over sodium sulfate and evaporated. The crude product is dissolved in 7.4 ml of 1,4-dioxane, and 36.2 ml of 0.1 N sodium hydroxide solution are added. After stirring at RT for 3 h, the reaction solution is evaporated, and the residue is taken up in water and ethyl acetate. The organic phase is extracted three times with ethyl acetate. The combined organic phases are dried over sodium sulfate and evaporated. 0.90 g (90% of theory) of the product is obtained.

Method B:

A solution of 14.0 g (38 mmol) of benzyl 2(S)-tert-butoxycarbonylamino-4(R)-hydroxy-5-nitropentanoate in 840 ml of ethanol/water 9/1 is mixed with 1.96 g of palladium on carbon (10%) and hydrogenated under atmospheric pressure at RT for 24 h. The mixture is filtered through kieselguhr, and the filtrate is mixed with 14.7 g (114 mmol) of diisopropylethylamine. Then 11.4 g (45.6 mmol) of N-(benzyloxycarbonyloxy)succinimide are added, and the mixture is stirred at RT for 4 h. The solution is concentrated, and the residue is taken up in dichloromethane and extracted twice with 0.1 N hydrochloric acid. The organic phase is separated off and made alkaline with 14.7 g (114 mmol) of diisopropylamine. The solution is cooled to 0° C., 30.1 g (114 mmol) of dimethyl-tert-butylsilyl trifluoromethanesulfonate are added, and the mixture is stirred at RT for 2.5 h. The organic phase is washed with saturated sodium bicarbonate solution, dried over sodium sulfate and evaporated. The residue is dissolved in 50 ml of dioxane, mixed with 200 ml of 0.1 N sodium hydroxide solution and stirred at RT for 3 h. After extraction several times with ethyl acetate, the collected organic phases are dried over sodium sulfate and concentrated in vacuo. The residue is chromatographed on silica gel (mobile phase: dichloromethane/ethanol 20/1, 9/1). 8.11 g (43% of theory) of the product are obtained.

MS (ESI): m/z=497 (M+H)⁺. ¹H-NMR (300 MHz, d₆-DMSO): δ=0.00 (s, 6H), 0.99 (s, 9H), 1.33 (s, 9H), 1.59 (m, 1H), 1.80 (m, 1H), 2.75-3.15 (m, 2H), 3.81 (m, 1H), 3.98 (m, 1H), 4.96 (m, 2H), 7.04 (d, 1H), 7.19 (m, 1H), 7.30 (m, 5H), 12.37 (br, s, 1H).

### Example 15A

### 2-(Trimethylsilyl)ethyl 3-[3′-(2(S)-amino-2-benzyloxycarbonylethyl)4,4′-bisbenzyloxybiphenyl-3-yl]-2(S)-benzyloxycarbonylaminopropionate hydrochloride

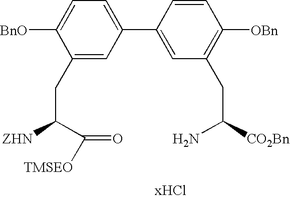

50 ml of a 4 M hydrochloric acid/dioxane solution are added over the course of about 20 min to a solution, cooled to 0° C., of 2.65 g (2.75 mmol) of 2-(trimethylsilyl)ethyl 2(S)-benzyloxycarbonylamino-3-[4,4′-bisbenzyloxy-3′-(2(S)-benzyloxycarbonyl-2-tert-butoxycarbonylaminoethyl)biphenyl-3-yl]propionate (Example 12A) in 50 ml of anhydrous dioxane. After stirring for 3 h, the reaction solution is evaporated and dried under high vacuum.

Yield: 100% of theory. HPLC (Method 3): Rₜ=5.96 min MS (EI): m/z=865 (M+H)⁺.

### Example 16A

### Benzyl 2(S)-[5-benzyloxycarbonylamino-2(S)-tert-butoxycarbonylamino-4(R)-(tert-butyldimethylsilyloxy)pentanoylamino]-3-{4,4′-bisbenzyloxy-3′-[2(S)-benzyloxycarbonylamino-2-(2-trimethylsilylethoxycarbonyl)ethyl]biphenyl-3-yl}propionate

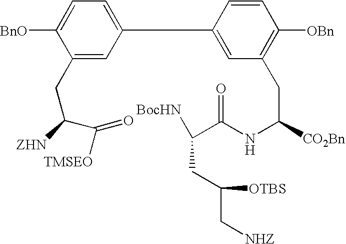

0.219 g (0.58 mmol) of HATU and 0.082 g (0.63 mmol) of N,N-diisopropylethylamine are added to a solution, cooled to 0° C. of 0.520 g (0.58 mmol) of (2-trimethylsilyl)ethyl 3-[3′-(2(S)-amino-2-benzyloxycarbonylethyl)-4,4′-bisbenzyloxybiphenyl-3-yl]-2(S)-benzyloxycarbonylaminopropionate hydrochloride (Example 15A) and 0.287 g (0.58 mmol) of 5-benzyloxycarbonylamino-2(S)-tert-butoxycarbonylamino-4(R)-(tert-butyldimethylsilyloxy)pentanoic acid (Example 14A) in 7.3 ml of anhydrous DMF. After stirring at 0° C. for 30 min, an additional 0.164 g (1.26 mmol) of N,N-diisopropylethylamine is added. The reaction mixture is stirred at RT for 15 h. The solvent is then evaporated, and the residue is taken up in ethyl acetate. The organic phase is washed three times with water and once with saturated aqueous sodium chloride solution, dried over sodium sulfate and concentrated. The crude product is purified by chromatography on silica gel with dichloromethane/ethyl acetate (gradient 30/1→20/1→10/1). 533 mg (66% of theory) of the product are obtained.

LC-MS (ESI, Method 12): m/z=1342 (M+H)⁺, 1365 (M+Na)⁺.

### Example 17A

### 2(S)-Benzyloxycarbonylamino-3-{4,4′-bisbenzyloxy-3′-[2(S)-benzyloxycarbonyl-2-(5-benzyloxycarbonylamino-2(S)-tert-butoxycarbonylamino-4(R)-hydroxypentanoylamino)ethyl]biphenyl-3-yl}propionic acid

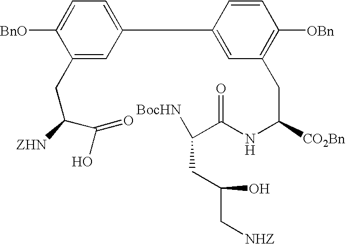 Method A:

0.80 ml of a 1.0 M solution of tetrabutylammonium fluoride in THF is added to a solution of 0.360 g (0.27 mmol) of benzyl 2(S)-[5-benzyloxycarbonylamino-2(S)-tert-butoxycarbonylamino-4(R)-(tert-butyldimethylsilyloxy)pentanoylamino]-3-{4,4′-bisbenzyloxy-3′-[2(S)-benzyloxycarbonylamino-2-(2-trimethylsilylethoxycarbonyl)ethyl]biphenyl-3-yl}propionate (Example 16A) in 22.5 ml of anhydrous DMF. After stirring at RT for 1 h, the reaction mixture is cooled to 0° C., and water is added. After addition of ethyl acetate, the phases are separated. The organic phase is washed with a 1.0 M solution of potassium bisulfate, dried over sodium sulfate and evaporated. 0.331 g of the crude product is obtained. The crude product is reacted without further purification.

LC-MS (ESI, Method 10): m/z=1129 (M+H)⁺. LC-HR-FT-ICR-MS: calc. for C₆₅H₆₉N₄O₁₄ (M+H)⁺1129.48048 found 1129.48123.

Method B:

1.8 ml of 1N tetrabutylammonium fluoride in THF are added dropwise to a solution of 800 mg (0.6 mmol) of benzyl 2(S)-[5-benzyloxycarbonylamino-2(S)-tert-butoxycarbonylamino-4(R)-(tert-butyldimethylsilyloxy)pentanoylamino]-3-{4,4′-bisbenzyloxy-3′-[2(S)-benzyloxycarbonylamino-2-(2-trimethylsilylethoxycarbonyl)ethyl]biphenyl-3-yl}propionate (Example 16A) in 26 ml of absolute DMF at RT. After 25 min at RT, the mixture is cooled to 0° C. and a large amount of ice-water is added. Ethyl acetate and some 1N hydrochloric acid solution are immediately added. The organic phase is dried with magnesium sulfate, concentrated and dried under high vacuum for 1 h. The crude product is reacted without further purification.

### Example 18A

### Benzyl 2(S)-(5-benzyloxycarbonylamino-2(S)-tert-butoxycarbonylamino-4(R)-hydroxypentanoylamino)-3-[4,4′-bisbenzyloxy-3′-(2(S)-benzyloxycarbonylamino-2-pentafluorophenyloxycarbonylethyl)biphenyl-3-yl]propionate

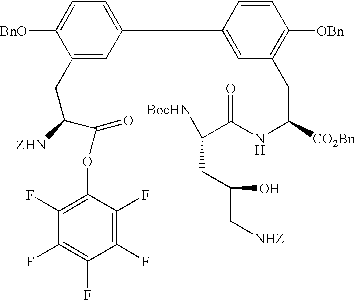 Method A:

90 mg of pentafluorophenol (0.49 mmol), dissolved in a little dichloromethane, and 1.1 mg of 4-dimethylaminopyridine (10 μM) and 19.4 mg (0.10 mmol) of EDC are added to a solution, cooled to −25° C., of 104 mg (92 μmol) of 2(S)-benzyloxycarbonylamino-3-{4,4′-bisbenzyloxy-3′-[2(S)-benzyloxycarbonyl-2-(5-benzyloxycarbonylamino-2(S)-tert-butoxycarbonylamino-4(R)-hydroxypentanoylamino)ethyl]biphenyl-3-yl}propionic acid (Example 17A) in 3 ml of dichloromethane under argon. After stirring for 15 h, the reaction mixture is concentrated. The crude product is reacted without further purification.

LC-MS (ESI, Method 11): m/z=1317 (M+Na)⁺, 1295 (M+H)⁺. LC-HR-FT-ICR-MS: calc. for C₇₁H₆₈F₅N₄O₁₄ (M+H)⁺1295.46467 found 1295.46430.

Method B:

691 mg (crude mixture, approx. 0.6 mmol) of 2(S)-benzyloxycarbonylamino-3-{4,4′-bisbenzyloxy-3′-[2(S)-benzyloxycarbonyl-2-(5-benzyloxycarbonylamino-2(S)-tert-butoxycarbonylamino-4(R)-hydroxypentanoylamino)ethyl]biphenyl-3-yl}propionic acid (Example 17A) are introduced into 25 ml of dichloromethane, and 547.6 mg (2.98 mmol) of pentafluorophenol, dissolved in 6 ml of dichloromethane, are added. 7.3 mg (0.06 mmol) of DMAP are added, and the mixture is cooled to −25° C. (ethanol/carbon dioxide bath). At −25° C., 148 mg (0.774 mmol) of EDC are added. The mixture slowly warms to RT overnight. The reaction mixture is concentrated in vacuo and briefly dried under high vacuum. The crude product is reacted without further purification.

### Example 19A

### Benzyl 5,17-bisbenzyloxy-14(S)-benzyloxycarbonylamino-11(S)-(3-benzyloxycarbonylamino-2(R)-hydroxypropyl)-10,13-dioxo-9,12-diazatricyclo[14.3.1.1^{2,6}]-henicosa-1(19),2,4,6(21),16(20),17-hexaene-8(S)-carboxylate

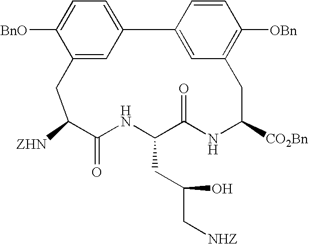 Method A:

4 ml of a 4 M hydrochloric acid solution in 1,4-dioxane are added to a solution of 119.3 mg of benzyl 2(S)-(5-benzyloxycarbonylamino-2(S)-tert-butoxycarbonylamino-4(R)-hydroxypentanoylamino)-3-[4,4′-bisbenzyloxy-3′-(2(S)-benzyloxycarbonylamino-2-pentafluorophenyloxycarbonylethyl)biphenyl-3-yl]propionate (Example 18A) in 2.7 ml of 1,4-dioxane. Until the reaction is complete, a further 1.5 ml of 4 M hydrochloric acid solution in 1,4-dioxane is added. The reaction solution is evaporated and codistilled with chloroform twice. The crude product (LC-HR-FT-ICR-MS, Method 13: calc. for C₆₆H₆₀F₅N₄O₁₂ (M+H)⁺1195.41224, found 1195.41419) is dissolved in 100 ml of chloroform and added dropwise over the course of 3 h to a very efficiently stirred suspension of 200 ml of chloroform and 100 ml of saturated aqueous sodium bicarbonate solution. The reaction mixture is vigorously stirred for 2 h. After the two phases have been separated, the aqueous phase is extracted with chloroform. The combined organic phases are washed with 5% strength aqueous citric acid solution, dried over magnesium sulfate and evaporated to dryness. The crude product is washed with acetonitrile and dried under high vacuum.

Yield: 60.5 mg (65% of theory) LC-MS (ESI, Method 11): m/z=1011 (M+H)⁺.

Method B:

About 0.595 mmol of benzyl 2(S)-(5-benzyloxycarbonylamino-2(S)-tert-butoxycarbonylamino-4(R)-hydroxypentanoylamino)-3-[4,4′-bisbenzyloxy-3′-(2(S)-benzyloxycarbonylamino-2-pentafluorophenyloxycarbonylethyl)biphenyl-3-yl]propionate (Example 18A) are dissolved in 8 ml of dioxane and then, at 0° C., 16 ml of 4 N hydrochloric acid solution in dioxane are added dropwise. After 45 min, 6 ml of 4 N hydrochloric acid solution in dioxane are again added, and after 15 min a further 8 ml are added. The mixture is stirred at 0° C. for 30 min before the reaction solution is concentrated under mild conditions, codistilled with chloroform (twice) and briefly dried under high vacuum. The crude product (732 mg, 0.59 mmol) is dissolved in 1000 ml of chloroform, and a solution of 6 ml of triethylamine in 50 ml of chloroform is added dropwise. The mixture is stirred at RT overnight. The mixture is worked up by evaporating under mild conditions in vacuo and stirring the residue in acetonitrile. The resulting crystals are filtered off with suction, washed with acetonitrile and dried under high vacuum.

Yield: 360 mg (60% of theory). BPLC (Method 3): Rₜ=5.59 min. ¹H-NMR (400 MHz, d₆-DMSO): δ=1.52-1.65 (m, 1H), 1.73-1.84 (ni, 1H), 2.82-3.01 (m, 31), 3.02-3.11 (m, 1H), 3.46 (s, 1H), 3.57-3.68 (m, 1H), 4.47-4.56 (m, 1H), 4.64-4.71 (m, 1H), 4.73-4.85 (m, 2H), 4.88-5.00 (m, 4H), 5.09 (s, 2H), 5.14-5.20 (m, 4H), 6.29 (d, 1H), 7.00-7.11 (m, 4H), 7.21-7.40 (m, 20H), 7.41-7.48 (m, 9H), 8.77 (d, 1H), 8.87 (d, 1H).

### Example 20A

### 14(S)-Amino-11(S)-(3-amino-2(R)-hydroxypropyl)-5,17-dihydroxy-10,13-dioxo-9,12-diazatricyclo[14.3.1.1^{2,6}]henicosa-1(19),2,4,6(21),16(20),17-hexaene-8(S)-carboxylic acid dihydrochloride

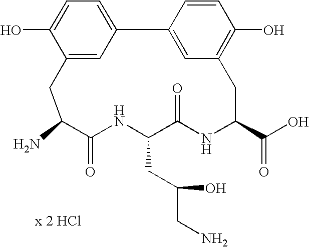 Method A:

A solution of 10 mg (9.9 μM) of benzyl 5,17-bisbenzyloxy-14(S)-benzyloxycarbonylamino-11(S)-(3-benzyloxycarbonylamino-2(R)-hydroxypropyl)-10,13-dioxo-9,12-diazatricyclo[14.3.1.1^{2,6}]henicosa-1(19),2,4,6(21),16(20),17-hexaene-8(S)-carboxylate (Example 19A) and 50 μl of formic acid in 10 ml of ethanol is vigorously stirred in the presence of 10 mg of Pd/C under hydrogen at atmospheric pressure for 16 h. The reaction solution is evaporated, and the residue is taken up in 1 N hydrochloric acid solution and filtered. The crude product is purified on an RP 18 cartridge with acetonitrile/water. 2 mg (42.8% of theory) of the product are obtained.

Method B:

200 mg (0.20 mmol) of benzyl 5,17-bisbenzyloxy-14(S)-benzyloxycarbonylamino-11(S)-(3-benzyloxycarbonylamino-2(R)-hydroxypropyl)-10,13-dioxo-9,12-diazatricyclo[14.3.1.1^{2,6}]henicosa-1(19),2,4,6(21),16(20),17-hexaene-8(S)-carboxylate (Example 19A) are put into 220 ml of an acetic acid/water/ethanol 4:1:1 mixture (ethanol can be replaced by THF). 73 mg of 10% palladium/carbon (10% Pd/C) are added, and then hydrogenation is carried out under atmospheric pressure for 15 h. The reaction mixture is filtered through prewashed kieselguhr, and the filtrate is concentrated in vacuo. The residue is mixed with 4.95 ml of 0.1 N aqueous hydrochloric acid and concentrated. The residue is stirred with 10 ml of diethyl ether and decantered. The remaining solid is dried under high vacuum.

Yield: 103 mg (95% of theory). HPLC (Method 3): Rₜ=3.04 min; LC-MS (Method 6): Rₜ=0.38 min MS (EI): m/z=473 (M+H)⁺. ¹H-NMR (400 MHz, D₂O): δ=2.06-2.20 (m, 1H), 2.74-2.89 (m, 1H), 2.94-3.05 (m, 1H), 3.12-3.25 (m, 2H), 3.53 (d, 1H), 3.61-3.72 (m, 1H), 3.97-4.07 (m, 1H), 4.53 (s, 1H), 4.61 (d, 1H), 4.76-4.91 (m, 12H), 7.01-7.05 (m, 2H), 7.07 (s, 1H), 7.40-7.45 (m, 2H), 7.51 (d, 1H).

### Example 21A

### (8S,11S,14S)-14-[(Tert-butoxycarbonyl)amino]-11-{(2R)-3-[(tert-butoxycarbonyl)amino]-2-hydroxypropyl}-5,17-dihydroxy-10,13-dioxo-9,12-diazatricyclo[14.3.1.1^{2,6}]henicosa-1(20),2(21),3,5,16,18-hexaene-8-carboxylic acid

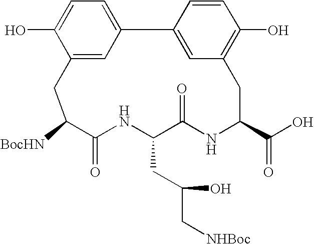 Method A:

5.2 mg (9.5 μmol) of 14(S)-amino-11(S)-(3-amino-2(R)-hydroxypropyl)-5,17-dihydroxy-10,13-dioxo-9,12-diazatricyclo[14.3.1.1^{2,6}]henicosa-1(19),2,4,6(21),16(20),17-hexaene-8(S)-carboxylic acid dihydrochloride (Example 20A) are dissolved in dry methanol (analytical grade, 0.5 ml) under argon. While stirring vigorously at room temperature, firstly an aqueous sodium bicarbonate solution (1 M, 100 μl) and then a methanolic solution of di-tert-butyl carbonate (0.1 M, 570 μl, 57 μmol) are added dropwise. Complete conversion is reached after about 1-2 days. The reaction mixture is evaporated in vacuo and dried under high vacuum. The resulting crude product is purified by gel chromatography [Sephadex LH-20; methanol/1 M sodium bicarbonate solution (1:0.0001)]. 5.3 mg (83% of theory) of product are obtained.

HPLC/UV-Vis (Method 14) Rₜ=7.4 min. λₘₐₓ (qualitative)=˜193 nm(s), 206 (sh), 269 (m), ˜284 (sh) (H₂O/acetonitrile+0.01% TFA [4:6]). LC-HR-FT-ICR-MS: calc. C₃₃H₄₄N₄O₁₁[M+H]⁺673.3079 found 673.3082.

Method B:

50 mg (0.09 mmol) of 14(S)-amino-11(S)-(3-amino-2(R)-hydroxypropyl)-5,17-dihydroxy-10,13-dioxo-9,12-diazatricyclo[14.3.1.1^{2,6}]henicosa-1(19),2,4,6(21),16(20),17-hexaene-8(S)-carboxylic acid dihydrochloride (Example 20A) are introduced into 8 ml of a methanol/water (9:1) mixture. To this are added 1 ml of 1 N sodium bicarbonate solution and then 80 mg (0.37 mmol) of di-tert-butyl dicarbonate in 2 ml of methanol/water (9:1). The mixture is stirred at RT overnight. The solution is worked up by mixing with 60 ml of ethyl acetate and 30 ml of water. The organic phase is washed once with 0.1 normal hydrochloric acid, dried and concentrated in vacuo.

Yield: 49 mg (79% of theory). LC-MS (Method 9): Rₜ=2.56 min. MS (EI): m/z=673 (M+H)⁺.

### Example 22A

### tert-Butyl (2R)-3-[(8S,11S,14S)-8-(aminocarbonyl)-14-[(tert-butoxycarbonyl)-amino]-5,17-dihydroxy-10,13-dioxo-9,12-diazatricyclo[14.3.1.1^{2,6}]henicosa-1(20),2(21),3,5,16,18-hexaene-11-yl]-2-hydroxypropylcarbamate

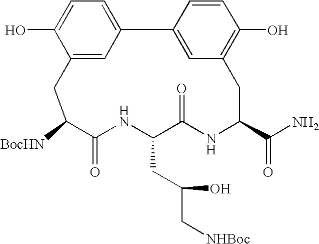 Method A:

4.1 mg (6.1 μmol) of (8S,11S,14S)-14-[(tert-butoxycarbonyl)amino]-11-{(2R)-3-[(tert-butoxycarbonyl)amino]-2-hydroxypropyl}-5,17-dihydroxy- 10,13-dioxo-9,12-diazatricyclo[14.3.1.1^{2,6}]henicosa-1(20),2(21),3,5,16,18-hexaene-8-carboxylic acid (Example 21A) are dissolved in dry N,N-dimethylformamide (analytical grade, 0.5 ml) under a protective atmosphere of argon gas. Addition of solid sodium disulfite (6.1 μmol) is followed by dropwise addition at RT of a freshly prepared solution of diisopropylethylamine (7.9 mg, 61 μmol), ammonium chloride (1.6 mg, 30 μmol) and HATU (4.6 mg, 12.2 μmol) in dimethylformamide (0.5 ml, solution A). Solution A must be added twice more (after a reaction time of 1.5 h and after a reaction time of 2 h) until conversion of precursor is complete. The mixture is stirred for a further 20 min, and then the reaction is stopped by adding water (0.5 ml). The reaction mixture is frozen and then freeze dried. The resulting crude product is purified by gel chromatography [Sephadex LH-20; methanol/acetic acid (1:0.0001) doped with sodium disulfite].

Yield: 2.2 mg (52% of theory). HPLC-UV-Vis (Method 14): Rₜ=7.06 min. λₘₐₓ (qualitative)=˜202 nm (s), 268 (m), ˜285 (sh), (H₂O/acetonitrile+0.01% TFA [4:6]). LC-HR-FT-ICR-MS (Method 13): calc. for C₃₃H₄₆N₅O₁₀ [M+H]⁺672.3239 found 672.3239.

Method B:

49 mg (0.07 mmol) of (8S,11S,14S)-14-[(tert-butoxycarbonyl)amino]-11-{(2R)-3-[(tert-butoxycarbonyl)amino]-2-hydroxypropyl}-5,17-dihydroxy-10,13-dioxo-9,12-diazatricyclo[14.3.1.1^{2,6}]henicosa-1(20),2(21),3,5,16,18-hexaene-8-carboxylic acid (Example 21A) are dissolved in 1 ml of DMF under argon and cooled to 0° C. Then 42 mg (0.11 mmol) of HATU are added, and the mixture is stirred at 0° C. for 10 min. 1.46 ml (0.73 mmol) of a 0.5 molar solution of ammonia in dioxane are added dropwise, and the mixture is stirred at RT overnight. After about 18 h, the same amounts of reagents are added once again. After 3 days, the mixture is concentrated in vacuo and purified by preparative RP-HPLC.

Yield: 16 mg (33% of theory). HPLC (Method 3): Rₜ=3.83 min.

### Example 23A

### tert-Butyl (2R)-3-[(8S,11S,14S)-8-[(benzylamino)carbonyl]-14-[(tert-butoxycarbonyl)amino]-5,17-dihydroxy-10,13-dioxo-9,12-diazatricyclo[14.3.1.1^{2,6}]-henicosa-1(20),2(21),3,5,16,18-hexaene-11-yl]-2-hydroxypropylcarbamate

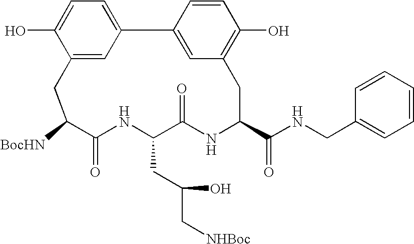

7.9 mg (0.021 mmol) of HATU are added to a solution, cooled to 0° C., of 7 mg (0.01 mmol) of ((8S,11S,14S)-14-[(tert-butoxycarbonyl)amino]-11-{(2R)-3-[(tert-butoxycarbonyl)amino]-2-hydroxypropyl}-5,17-dihydroxy-10,13-dioxo-9,12-diazatricyclo[14.3.1.1^{2,6}]henicosa-1(20),2(21),3,5,16,18-hexaene-8-carboxylic acid (Example 21A) in 0.5 ml of absolute DMF under argon. After 10 min at 0° C., 2.3 mg (0.021 mmol) of benzylamine are added, and the mixture is stirred at RT overnight. The reaction mixture is concentrated in vacuo, and the residue is separated by preparative RP-HPLC.

Yield: 1.5 mg (18.9% of theory). LC-MS (Method 6): Rₜ=4.4 min. MS (ESI-pos): m/z=785 (M+Na)⁺, 762 (M+H)⁺.

### Example 24A

### tert-Butyl (2R)-3-[(8S,11S,14S)-14-[(tert-butoxycarbonyl)amino]-5,17-dihydroxy-8-{[(2-hydroxyethyl)(methyl)amino]carbonyl}-10,13-dioxo-9,12-diazatricyclo[14.3.1.1^{2,6}]henicosa-1(20),2(21),3,5,16,18-hexaene-11-yl]-2-hydroxypropylcarbamate

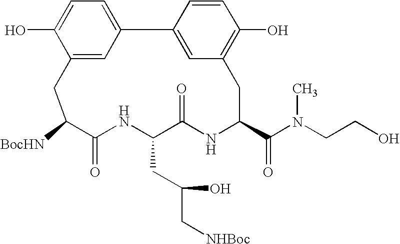

15 mg (0.022 mmol) of (8S,11S,14S)-14-[(tert-butoxycarbonyl)amino]-1-{(2R)-3-[(tert-butoxycarbonyl)amino]-2-hydroxypropyl}-5,17-dihydroxy- 10,13-dioxo-9,12-diazatricyclo[14.3.1.1^{2,6}]henicosa-1(20),2(21),3,5,16,18-hexaene-8-carboxylic acid (Example 21A) are dissolved in 0.5 ml of DMF under argon and cooled to 0° C. 10.2 mg (0.027 mmol) of HATU and 8.64 mg (0.067 mmol) of N,N-diisopropylethylamine are added thereto, and the mixture is stirred at 0° C. for 10 min. 3.34 mg (0.045 mmol) of 2-methylaminoethanol are added, and the mixture is stirred at RT overnight. The reaction mixture is concentrated and purified by Gilson HPLC.

Yield: 3.8 mg (23% of theory). LC-MS (Method 21): Rₜ=3.90 min.

Examples 25A to 32A listed in the following table can be prepared in analogy to Example 24A.

| Example | | Analytical |
|---|---|---|
| No. | Structure | data |
| 25A | 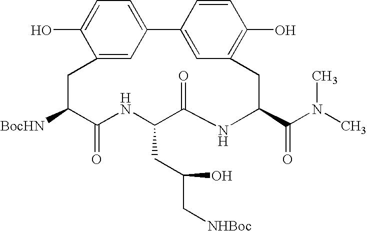 | HPLC (Method 3): Rₜ = 3.15 min. |
| 26A | 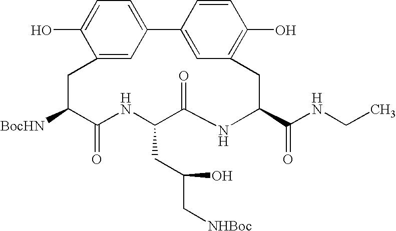 | HPLC (Method 3): Rₜ = 3.18 min. |
| 27A | 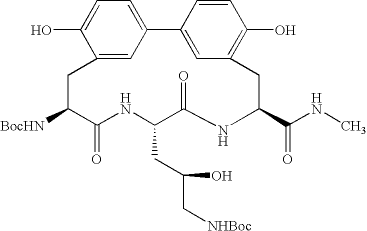 | HPLC (Method 3): Rₜ = 3.10 min. |
| 28A | 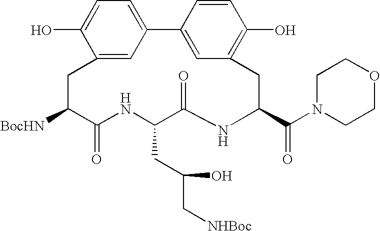 | LC-MS (Method 21): Rₜ = 3.97 min. |
| 29A | 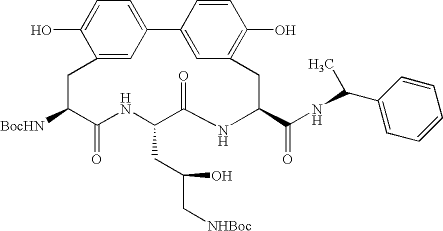 | HPLC (Method 4): Rₜ = 4.15 min. |
| 30A | 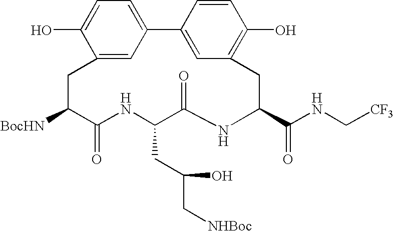 | HPLC (Method 3): Rₜ = 3.42 min. |
| 31A | 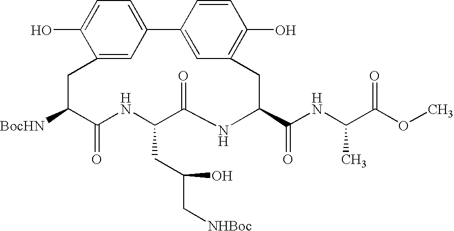 | LC-MS (Method 15): Rₜ = 2.18 min. MS (EI): m/z = 834 (M + H)⁺ |
| 32A | 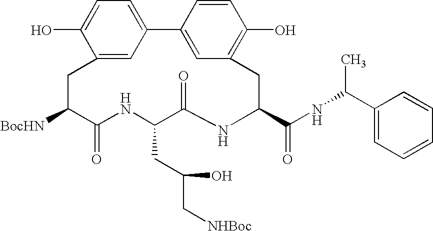 | HPLC (Method 4): Rₜ = 4.16 min. |

Examples 33A and 34A listed in the following table can be prepared in analogy to Example 24A using 2 eq of HATU and 3 eq of amine.

| Example | | Analytical |
|---|---|---|
| No. | Structure | data |
| 33A | 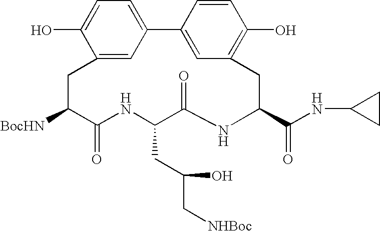 | HPLC (Method 3): Rₜ = 3.18 min. |
| 34A | 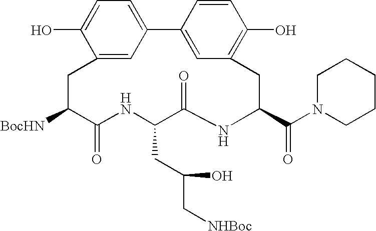 | HPLC (Method 3): Rₜ = 3.37 min. |

Examples 35A and 36A listed in the following table can be prepared in analogy to Example 24A using 2 eq of HATU, 2 eq of amine and without addition of DIPEA.

| Example | | Analytical |
|---|---|---|
| No. | Structure | data |
| 35A | 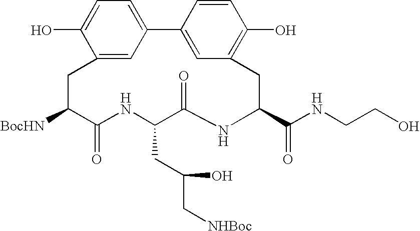 | HPLC (Method 3): Rₜ = 3.04 min. |
| 36A | 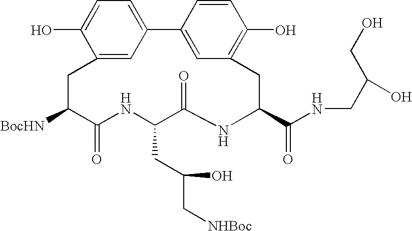 | HPLC (Method 1): Rₜ = 1.75 min. |

### Example 37A

### Benzyl 2-(benzyloxy)-N-(tert-butoxycarbonyl)-5-iodo-L-phenylalanyl-L-phenylalaninate

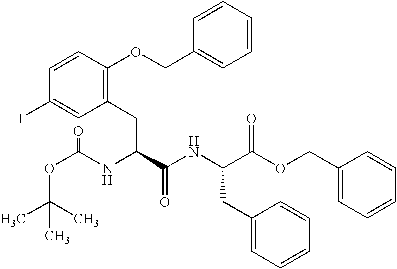

0.4 g (0.8 mmol) of 2-(benzyloxy)-N-(tert-butoxycarbonyl)-5-iodo-L-phenylalanine (Example 6A) and 0.282 g (0.970 mmol, 1.2 eq) of L-phenylalanine benzyl ester hydrochloride are introduced into 6 ml of DMF under argon and, at RT, 0.382 g (1.01 mmol, 1.25 eq) of HATU and 0.49 ml (0.36 mg, 2.8 mmol, 3.5 eq) of diisopropylethylamine are successively added. The mixture is stirred at RT for 12 hours. After addition of 150 ml of water, the product separates out in the form of white crystals. The crystals are filtered off with suction, washed with water and dried in vacuo.

Yield: 0.669 g (quant.) LC-MS (Method 15): Rₜ=3.11 min. MS (EI): m/z=735 (M+H)⁺

Examples 38A to 41A listed in the following table can be prepared in analogy to Example 37A.

| Example | | Analytical |
|---|---|---|
| No. | Structure | data |
| 38A | 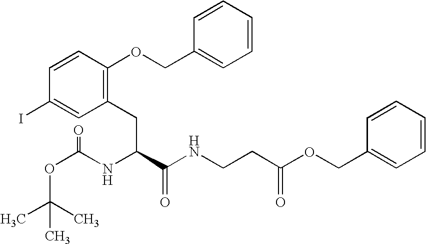 | LC-MS (Method 15): Rₜ = 2.86 min. MS (EI): m/z = 659 (M + H)⁺ |
| 39A | 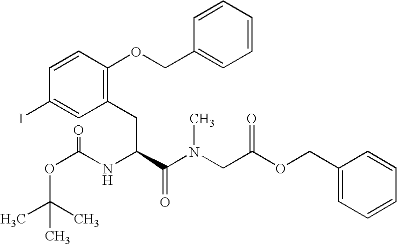 | LC-MS (Method 15): Rₜ = 2.96 min. MS (EI): m/z = 659 (M + H)⁺ |
| 40A | 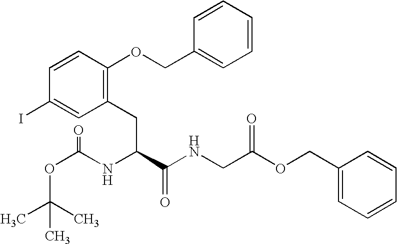 | LC-MS (Method 15): Rₜ = 2.85 min. MS (EI): m/z = 644 (M + H)⁺ |
| 41A | 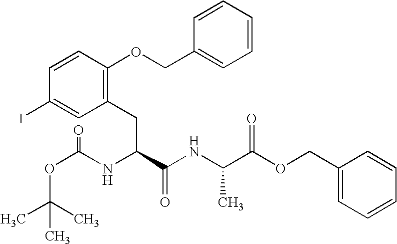 | LC-MS (Method 15): Rₜ = 2.93 min. MS (EI): m/z = 659 (M + H)⁺ |

### Example 42A

### 2-(Trimethylsilyl)ethyl 2-(S)-benzyloxycarbonylamino-3-[3′[-2-[tert-butoxycarbonylamino(3-amino-[1-(S)-benzyloxy-1-oxo-2-phenylethyl]-3-oxopropyl)]-4,4′-bis(benzyloxy)-1,1′-biphenyl-3-yl]]propanoate

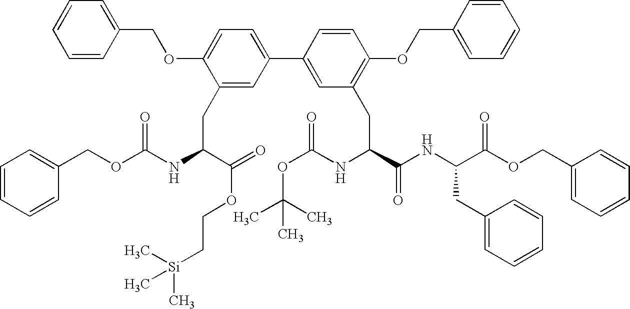

0.593 g (0.939 mmol) of 2-(trimethylsilyl)ethyl 2-(benzyloxy)-N-[(benzyloxy)carbonyl]-5-(4,4,5,5-tetramethyl-1,3,2-dioxaborolan-2-yl)-L-phenylalaninate (Example 84A) and 0.734 g (0.939 mmol) of benzyl 2-(benzyloxy)-N-(tert-butoxycarbonyl)-5-iodo-L-phenylalanyl-L-phenylalaninate (Example 37A) are dissolved in 6 ml of DMSO under argon. The resulting solution is flushed with argon for 30 min. Then 0.069 g (0.094 mmol, 0.1 eq) of bis(diphenylphosphino)ferrocenepalladium(II) chloride and 0.612 g (1.88 mmol, 2.0 eq) of cesium carbonate are added. After flushing with argon for 10 minutes, the mixture is heated at 80° C. for 3 days, continuing to flush with argon. After cooling to RT, the crude solution is purified by chromatography on silica gel (cyclohexane/ethyl acetate 2:1). The concentrated product-containing fractions are then purified by preparative RP-HPLC.

Yield: 0.367 g (29% of theory) LC-MS (Method 15): Rₜ=3.50 min.

Examples 43A to 46A listed in the following table can be prepared in analogy to Example 42A.

| Example | | Analytical |
|---|---|---|
| No. | Structure | data |
| 43A | 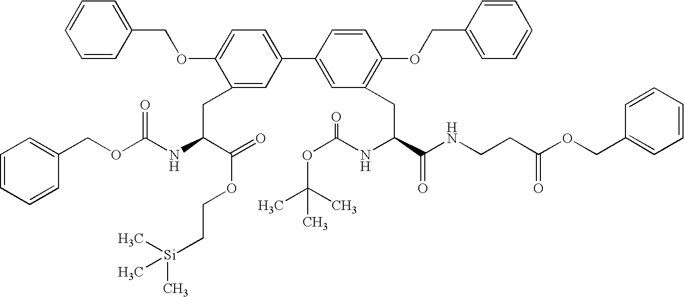 | LC-MS (Method 15): Rₜ = 3.39 min. MS (EI): m/z = 1036 (M + H)⁺ |
| 44A | 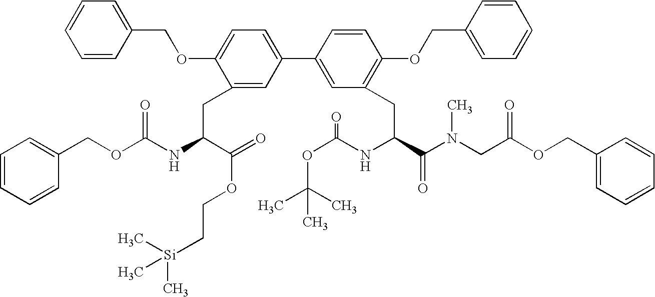 | LC-MS (Method 15): Rₜ = 3.42 min. MS (EI): m/z = 1036 (M + H)⁺ |
| 45A | 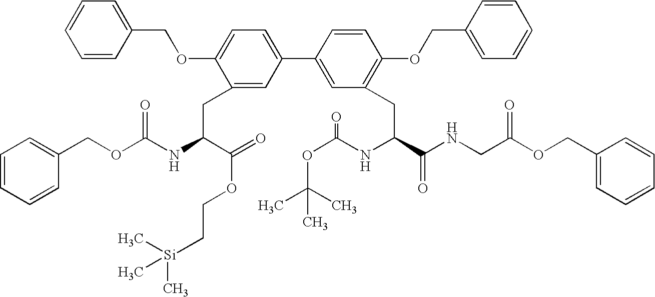 | LC-MS (Method 15): Rₜ = 3.38 min. MS (EI): m/z = 1022 (M + H)⁺ |
| 46A | 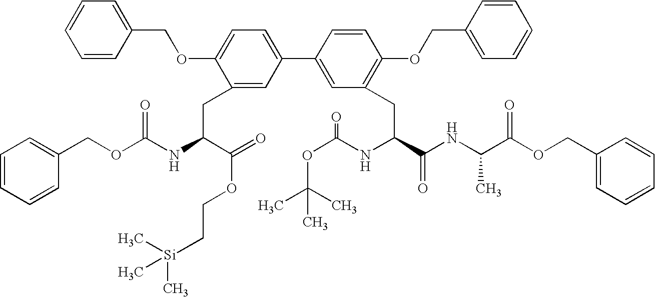 | LC-MS (Method 15): Rₜ = 3.40 min. MS (EI): m/z = 1036 (M + H)⁺ |

### Example 47A

### 2-(Trimethylsilyl)ethyl 2-(S)-benzyloxycarbonylamino-3-[3′[-2-[amino(3-amino-[1-(S)-benzyloxy-1-oxo-2-phenylethyl]-3-oxopropyl)]-4,4′-bis(benzyloxy)-1,1′-biphenyl-3-yl]]propanoate

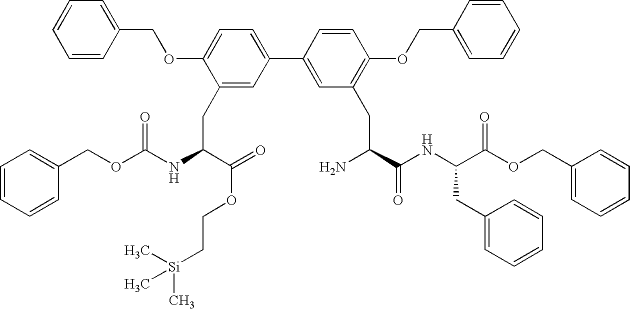

0.37 g (0.27 mmol) of 2-(trimethylsilyl)ethyl 2-(S)-benzyloxycarbonylamino-3-[3′[-2-[tert-butoxycarbonylamino(3-amino-[1-(S)-benzyloxy-1-oxo-2-phenylethyl]-3-oxopropyl)]-4,4′-bis(benzyloxy)-1,1′-biphenyl-3-yl]]propanoate (Example 42A) is dissolved in 10 ml of a 4 M solution of hydrogen chloride in dioxane under argon and stirred at RT for 3 h. The solution is concentrated in a rotary evaporator and dried in vacuo. The crude product is reacted further without further characterization.

Examples 48A to 51A listed in the following table can be prepared in analogy to Example 47A.

| Example | |
|---|---|
| No. | Structure |
| 48A | 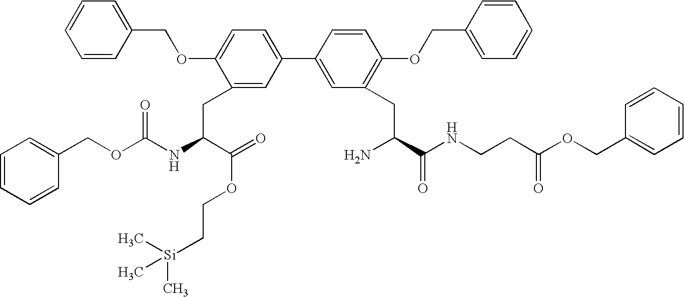 |
| 49A | 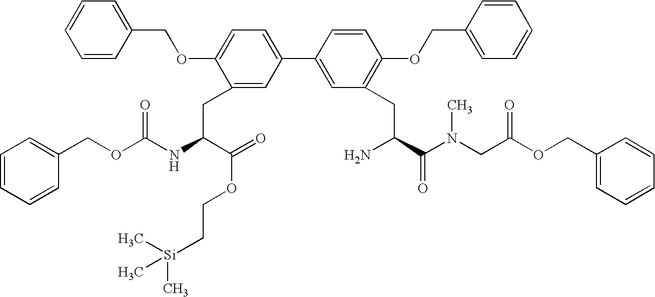 |
| 50A | 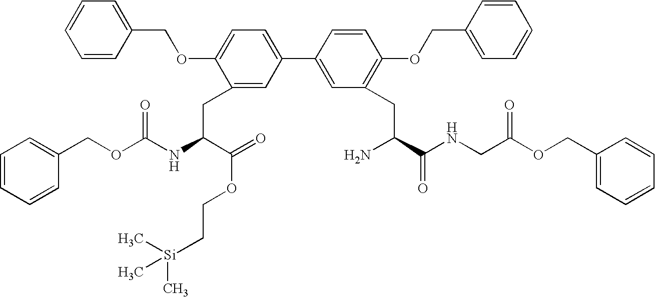 |
| 51A | 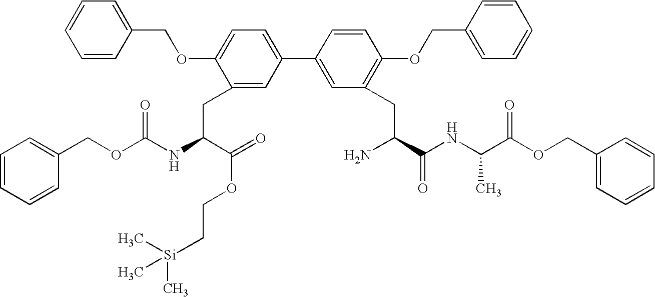 |

### Example 52A

### 2-(Trimethylsilyl)ethyl 2-(S)-benzyloxycarbonylamino-3-[3′[-2-[5-benzyloxycarbonylamino-2(S)-tert-butoxycarbonylamino-4(R)-(tert-butyldimethyl-silyloxy)pentanoylamino(3-amino-[1-(S)-benzyloxy-1-oxo-2-phenylethyl]-3-oxopropyl)]-4,4′-bis(benzyloxy)-1,1′-biphenyl-3-yl]]propanoate

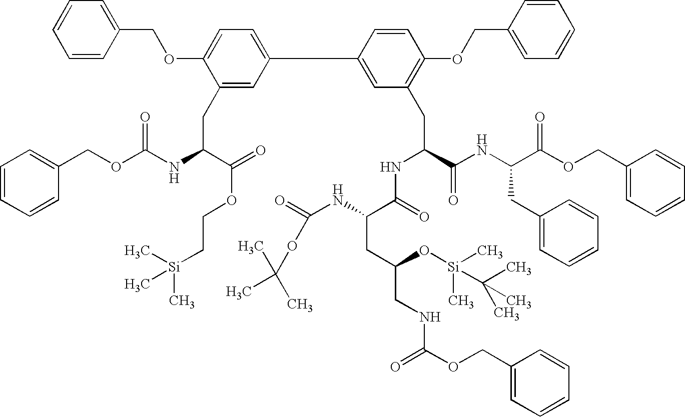

0.27 g (0.27 mmol) of 2-(trimethylsilyl)ethyl 2-(S)-benzyloxycarbonylamino-3-[3′[-2-[amino(3-amino-[1-(S)-benzyloxy-1-oxo-2-phenylethyl]-3-oxopropyl)]-4,4′-bis(benzyloxy)-1,1′-biphenyl-3-yl]]propanoate (Example 47A) and 0.16 g (0.32 mmol, 1.2 eq) of 5-benzyloxycarbonylamino-2(S)-tert-butoxycarbonylamino-4(R)-(tert-butyldimethylsilyloxy)pentanoic acid are dissolved in 5 ml of anhydrous DMF under argon. At RT, 0.13 g (0.34 mmol, 1.25 eq) of HATU and 0.16 ml (0.12 g, 0.95 mmol, 3.5 eq) of N,N-diisopropylethylamine are added. The reaction mixture is stirred at RT for 12 h. The reaction mixture is purified directly by preparative RP-HPLC and is reacted without further characterization.

Yield: 0.288 g (71% of theory).

Example 53A to 56A listed in the following table can be prepared in analogy to Example 52A.

| Example | | Analytical |
|---|---|---|
| No. | Structure | data |
| 53A | 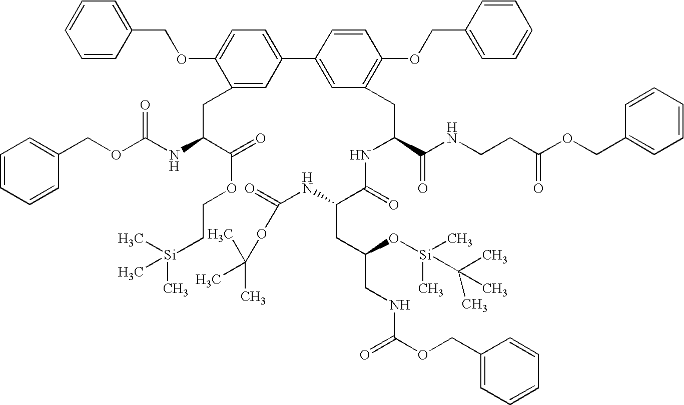 | LC-MS (Method 15): Rₜ = 3.84 min. MS (EI): m/z = 1415 (M + H)⁺ |
| 54A | 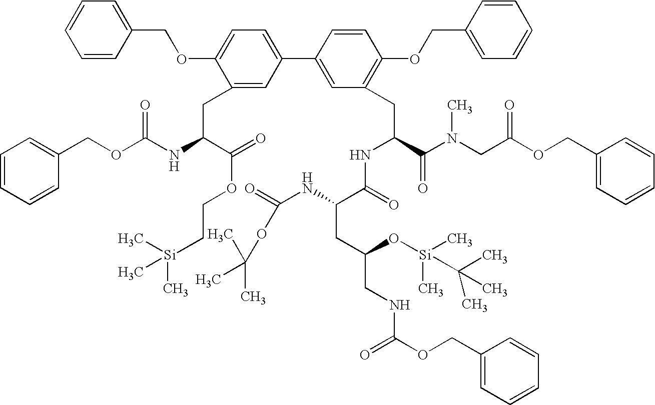 | LC-MS (Method 15): Rₜ = 3.92 min. MS (EI): m/z = 1415 (M + H)⁺ |
| 55A | 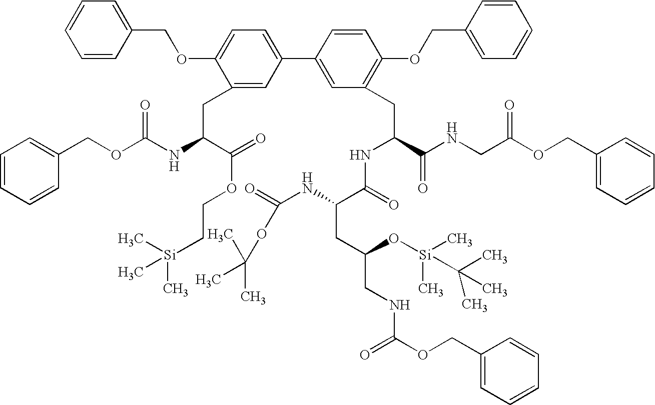 | LC-MS (Method 15): Rₜ = 3.97 min. MS (EI): m/z = 1401 (M + H)⁺ |
| 56A | 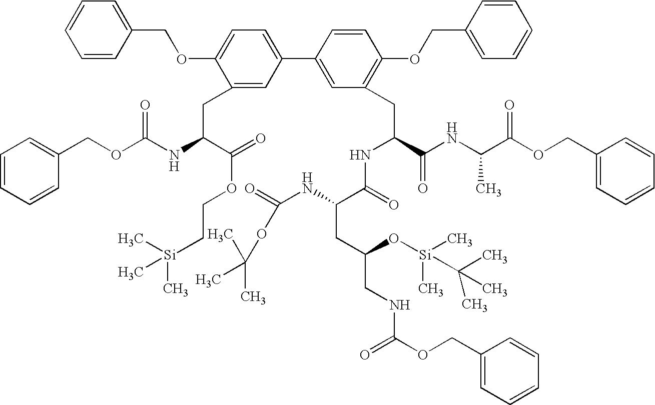 | LC-MS (Method 16): Rₜ = 2.98 min. MS (EI): m/z = 1415 (M + H)⁺ |

### Example 57A

### 2-(S)-Benzyloxycarbonylarnino-3-[3′[-2-[5-benzyloxycarbonylamino-2(S)-tert-butoxycarbonylamino-4(R)-(hydroxyoxy)pentanoylamino(3-amino-[1-(S)-benzyloxy-1-oxo-2-phenylethyl]-3-oxopropyl)]-4,4′-bis(benzyloxy)-1,1′-biphenyl-3-yl]]propionic acid

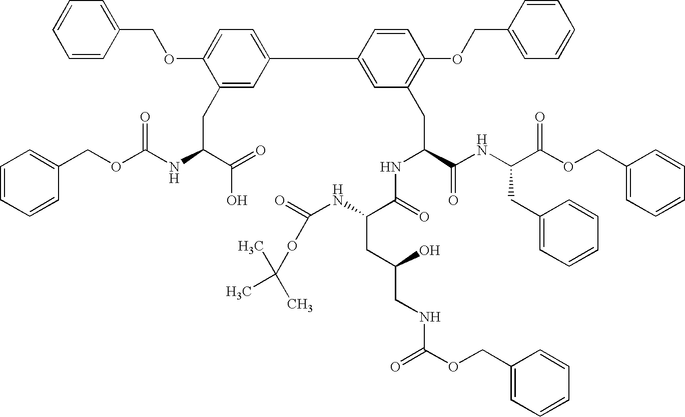

1.2 ml of a 1.0 M solution of tetrabutylammonium fluoride in THF (1.2 mmol, 6.3 eq) are added to a solution of 0.29 g (0.19 mmol) of 2-(trimethylsilyl)ethyl 2-(S)-benzyloxycarbonylamino-3[3′[-2-[5-benzyloxycarbonylamino-2(S)-tert-butoxycarbonylamino-4(R)-(tert-butyldimethylsilyloxy)pentanoylamino(3-amino-[1-(S)-benzyloxy-1-oxo-2-phenyl ethyl]-3-oxopropyl)]-4,4′-bis(benzyloxy)-1,1′-biphenyl-3-yl]]propanoate (Example 52A) in 3 ml of DMF. After stirring at RT for 4 h, the reaction mixture is cooled to 0° C., and 50 ml of water are added. After addition of 50 ml of ethyl acetate and 1 ml of 1 N aqueous hydrochloric acid, the phases are separated. The aqueous phase is extracted several times with ethyl acetate. After the organic phase has been dried over magnesium sulfate it is concentrated in vacuo and dried under high vacuum. The crude product is reacted without further purification.

Examples 58A to 61A listed in the following table can be prepared in analogy to Example 57A.

| Example | |
|---|---|
| No. | Structure |
| 58A | 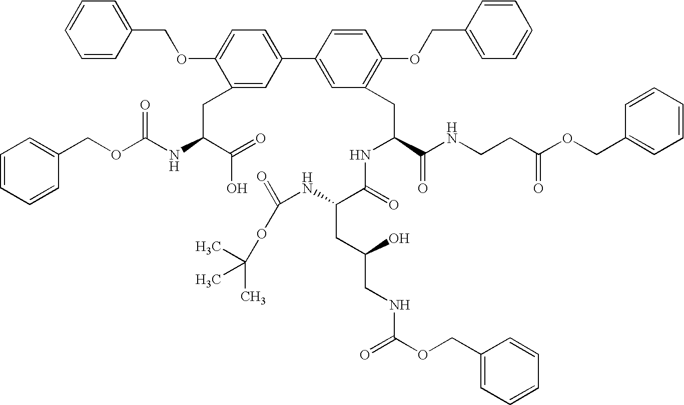 |
| 59A | 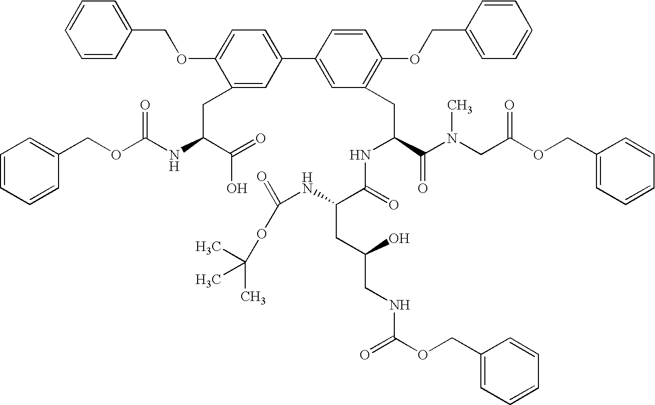 |
| 60A | 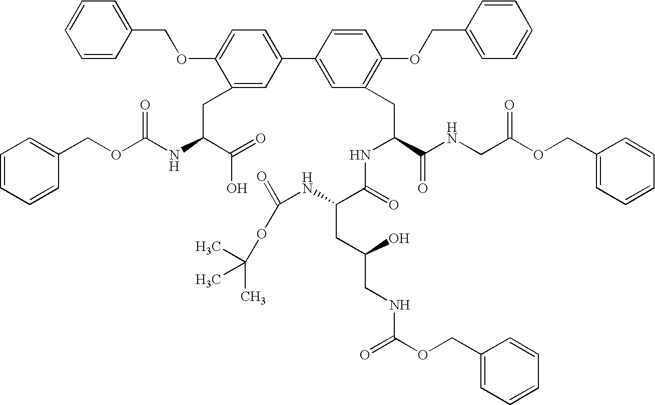 |
| 61A | 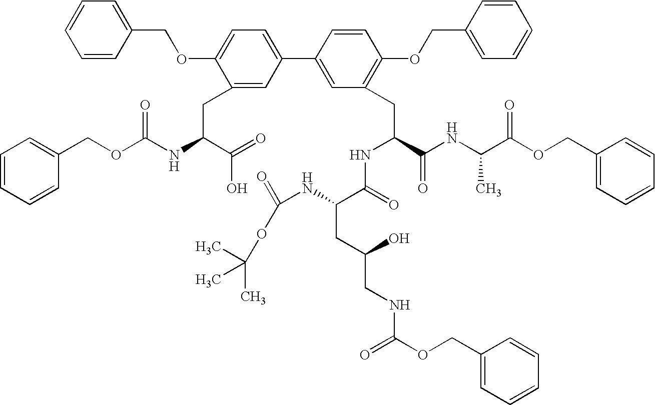 |

### Example 62A

### Pentafluorophenyl 2-(S)-benzyloxycarbonylamino-3-[3′[-2-[5-benzyloxycarbonylamino-2(S)-tert-butoxycarbonylamino-4(R)-(hydroxyoxy)pentanoylamino(3-amino-[1-(S)-benzyloxy-1-oxo-2-phenylethyl]-3-oxopropyl)]-4,4′-bis(benzyloxy)-1,1′-biphenyl-3-yl]]propionate

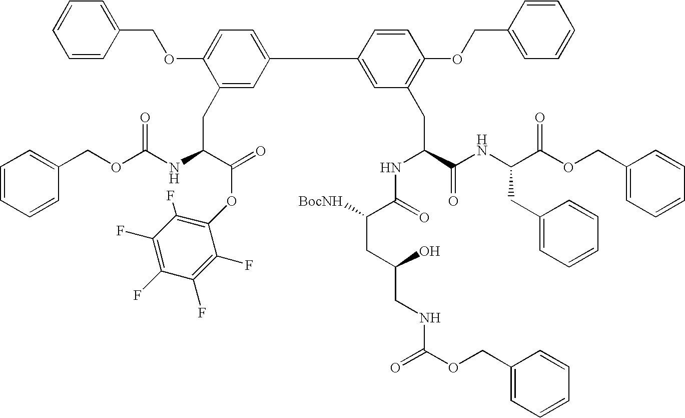

0.25 g (crude mixture, about 0.19 mmol) of 2-(S)-benzyloxycarbonylamino-3-[3′[-2-[5-benzyloxycarbonylamino-2(S)-tert-butoxycarbonylamino-4(R)-(hydroxyoxy)pentanoylamino(3-amino-[1-(S)-benzyloxy-1-oxo-2-phenylethyl]-3-oxopropyl)]-4,4′-bis(benzyloxy)-1,1′-biphenyl-3-yl]]propionic acid (Example 57A) are introduced into 4 ml of DCM, and 0.18 g (0.97 mmol, 5.0 eq) of pentafluorophenol and 0.02 g (0.02 mmol, 0.1 eq) of DMAP are added. The mixture is cooled to −25° C., and 0.048 g (0.25 mmol, 1.3 eq) of EDC is added. The mixture is slowly warmed to RT overnight. The reaction mixture is concentrated in vacuo and briefly dried under high vacuum. The crude product is reacted without further purification.

Examples 63A to 66A listed in the following table can be prepared in analogy to Example 62A.

| Example | |
|---|---|
| No. | Structure |
| 63A | 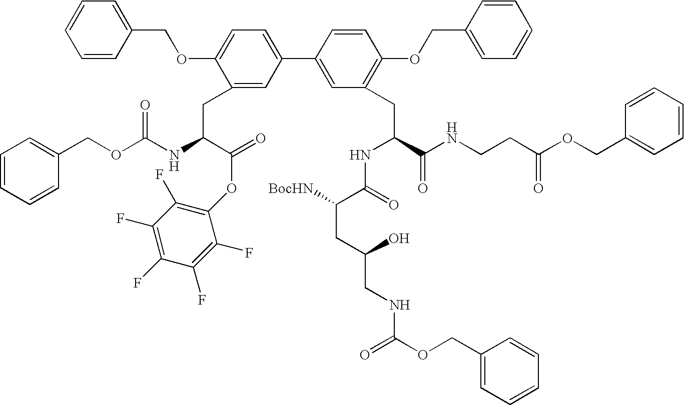 |
| 64A | 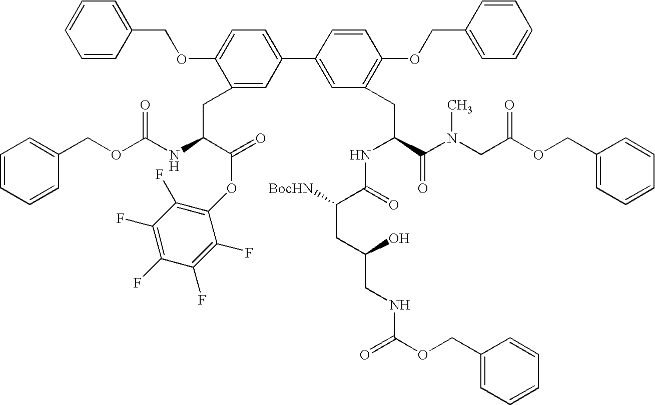 |
| 65A | 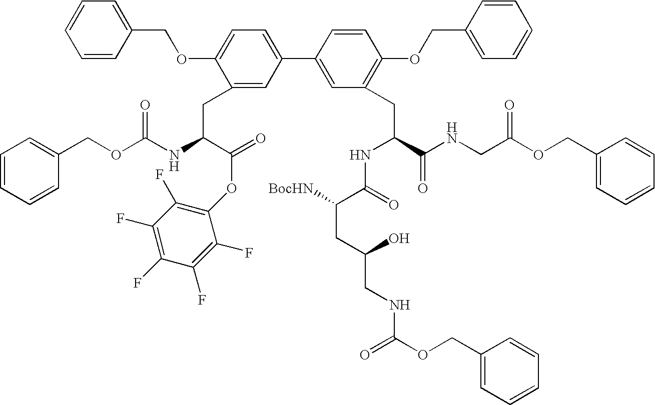 |
| 66A | 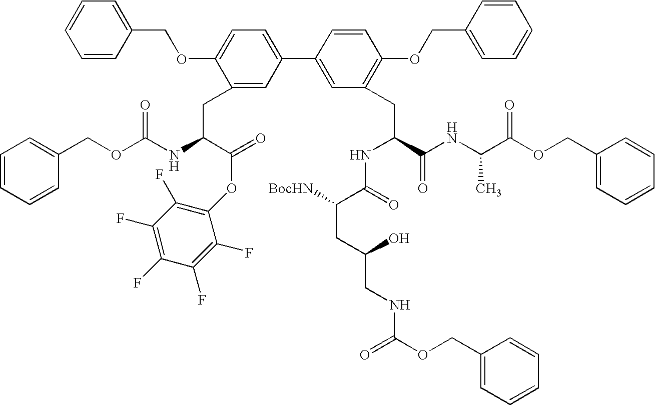 |

### Example 67A

### Pentafluorophenyl 2-(S)-benzyloxycarbonylamino-3-[3′[-2-[5-benzyloxycarbonylamino-2(S)-amino-4(R)-(hydroxyoxy)pentanoylamino(3-amino-[1-(S)-benzyloxy-1-oxo-2-phenylethyl]-3-oxopropyl)]-4,4′-bis(benzyloxy)-1,1′-biphenyl-3-yl]]propionate

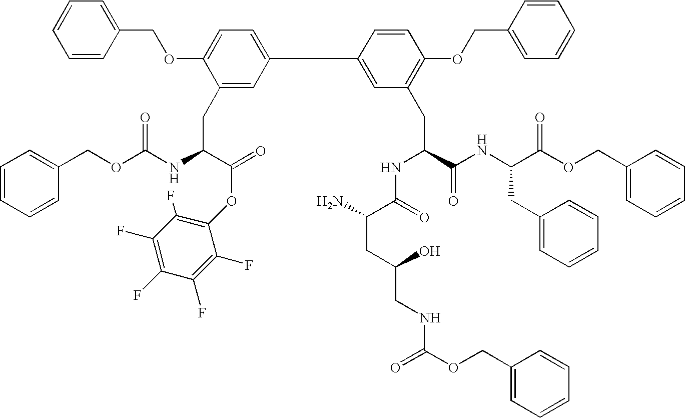

0.28 g (0.19 mmol) of pentafluorophenyl 2-(S)-benzyloxycarbonylamino-3-[3′[-2-[5-benzyloxycarbonylamino-2(S)-tert-butoxycarbonylamino-4(R)-(hydroxyoxy)pentanoylamino(3-amino-[1-(S)-benzyloxy-1-oxo-2-phenylethyl]-3-oxopropyl)]-4,4′-bis(benzyloxy)-1,1′-biphenyl-3-yl]]propionate (Example 62A) are dissolved in 4 ml of a 4 M hydrogen chloride solution in dioxane at RT. After 3 h at RT, the reaction solution is concentrated at 30° C. in vacuo and dried under high vacuum. The crude product is reacted without further purification.

Examples 68A to 71A listed in the following table can be prepared in analogy to Example 67A.

| Example | |
|---|---|
| No. | Structure |
| 68A | 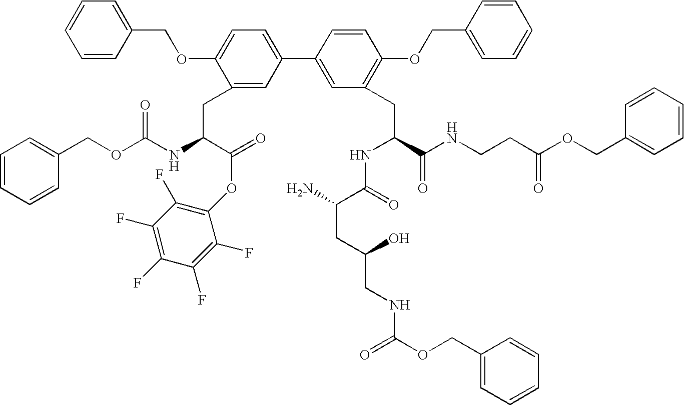 |
| 69A | 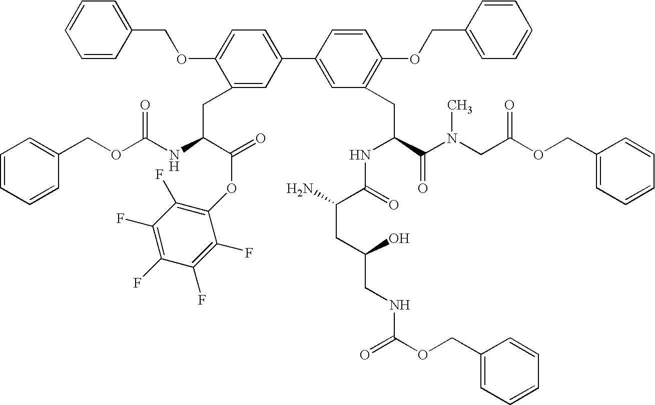 |
| 70A | 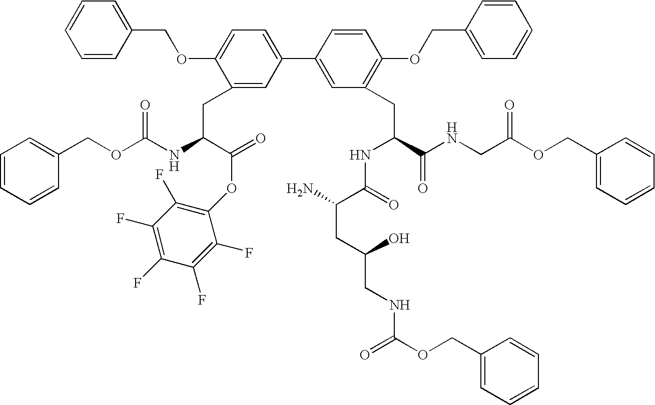 |
| 71A | 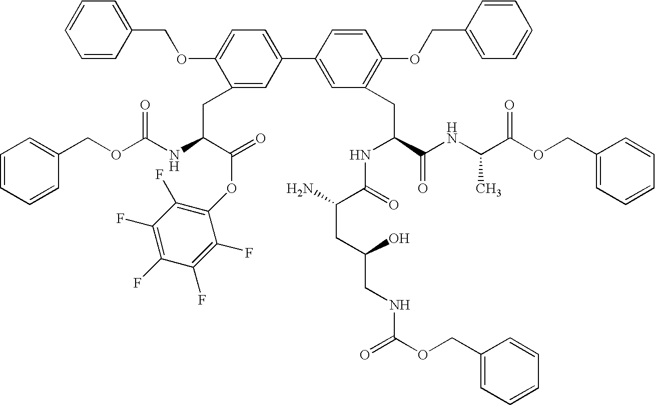 |

### Example 72A

### Benzyl N-{[(8S,11S,14S)-5,17-bis(benzyloxy)-14-{[(benzyloxy)carbonyl]amino}-11-((2R)-3-{[(benzyloxy)carbonyl]amino}-2-hydroxypropyl)-10,13-dioxo-9,12-diazatricyclo[14.3.1.1^{2,6}]henicosa-1(20),2(21),3,5,16,18-hexaene-8-yl]carbonyl}-L-phenylalaninate

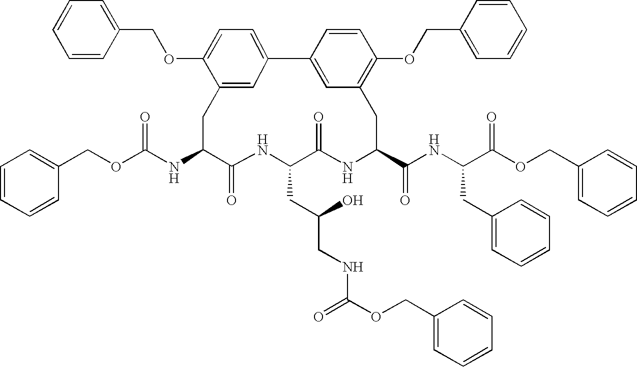

0.26 g (0.19 mmol) of pentafluorophenyl 2-(S)-benzyloxycarbonylamino-3-[3′[-2-[5-benzyloxycarbonylamino-2(S)-amino-4(R)-(hydroxyoxy)pentanoylamino(3-amino-[1-(S)-benzyloxy-1-oxo-2-phenylethyl]-3-oxopropyl)]-4,4′-bis(benzyloxy)-1,1′-biphenyl-3-yl]]propionate (Example 67A) are dissolved in 200 ml of chloroform and added dropwise over the course of 4 h to a solution of 2000 ml of chloroform and saturated aqueous sodium bicarbonate solution at RT. Stirring is continued for 1 h after addition is complete. The phases are then separated. The aqueous phase is washed twice with 500 ml of DCM. The combined organic phases are washed with 2000 ml of 0.1 M aqueous hydrochloric acid, dried over sodium sulfate and concentrated in vacuo. The residue is suspended in 15 ml of acetonitrile:mnethanol (2:1) and stirred at RT for 1 h. The undissolved solid is filtered off and dried in vacuo. The solid is boiled in methanol for 15 min for further purification. The product is obtained by renewed filtration and drying in vacuo.

Yield: 0.022 g (10% of theory). LC-MS (Method 15): Rₜ=3.13 min. MS (EI): m/z=1158 (M+H)⁺

Examples 73A to 76A listed in the following table can be prepared in analogy to Example 72A.

| Example | | Analytical |
|---|---|---|
| No. | Structure | data |
| 73A | 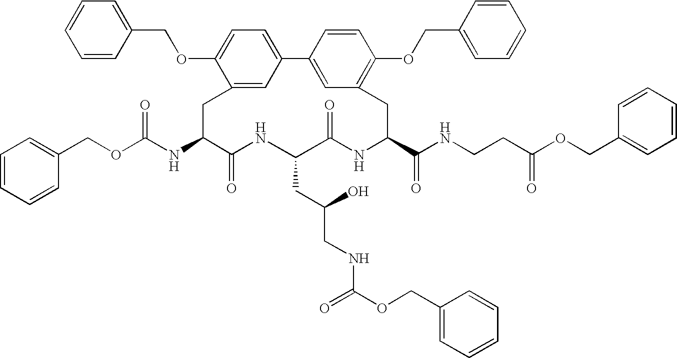 | LC-MS (Method 15): Rₜ = 2.97 min. MS (EI): m/z = 1082 (M + H)⁺ |
| 74A | 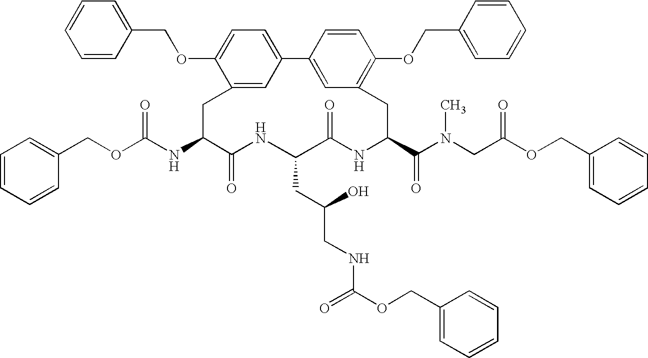 | LC-MS (Method 15): Rₜ = 3.00 min. MS (EI): m/z = 1082 (M + H)⁺ |
| 75A | 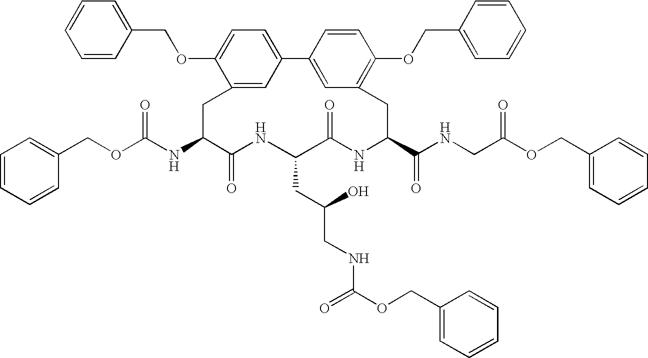 | LC/MS (Method 15): Rₜ = 2.94 min. MS (EI): m/z = 1068 (M + H)⁺ |
| 76A | 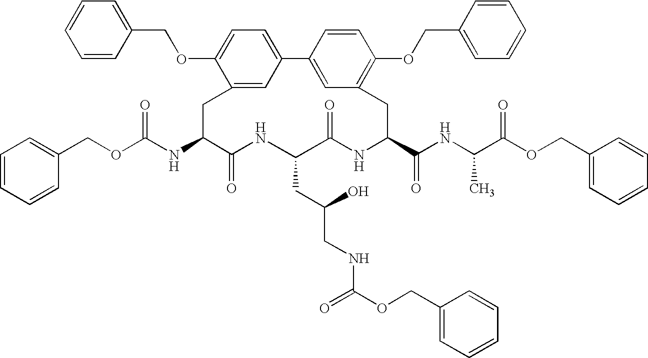 | LC/MS (Method 15): Rₜ = 2.95 min. MS (EI): m/z = 1083 (M + H)⁺ |

### Example 77A

### Benzyl 2(S)-[S-benzyloxycarbonylamino-2(S)-tert-butoxycarbonylamino-pentanoylamino]-3-{4,4′-bisbenzyloxy-3′-[2(S)-benzyloxycarbonylamino-2-(2-trimethylsilylethoxycarbonyl)ethyl]-biphenyl-3-yl}propionate

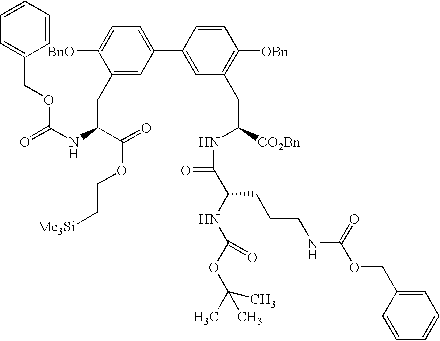

Preparation takes place in analogy to Example 16A from 0.47 g (0.51 mmol) of the compound from Example 15A and 0.19 g (0.51 mmol) of N_{α}-Boc-N_{δ}-Z-L-ornithine with 0.19 g (0.51 mmol) of HATU and 0.35 ml (1.65 mmol) of N,N-diisopropylethylamine in 5.55 ml of dry DMF.

Yield: 0.58 g (92% of theory). LC-MS (Method 18): Rₜ=3.46 min. MS: m/z=1212 (M+H)⁺

### Example 78A

### 2(S)-Benzyloxycarbonylamino-3-{4,4′-bisbenzyloxy-3′-[2(S)-benzyloxycarbonyl-2-(5-benzyloxycarbonylamino)-2(S)-tert-butoxycarbonylaminopentanoylamino)ethyl]biphenyl-3-yl}propionic acid

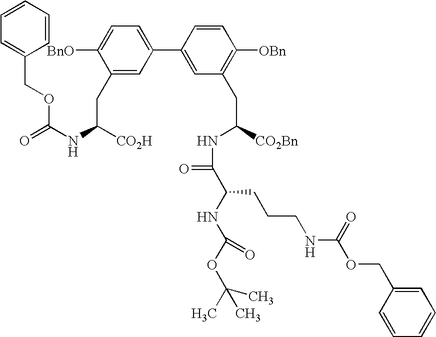

Preparation takes place in analogy to Example 17A from 0.82 g (0.68 mmol) of the compound from Example 77A with 2 eq (1.3 ml) of tetrabutylammonium fluoride (1 M in THF) in 30 ml dry DMF.

Yield: 772 mg (94% of theory). LC-MS (Method 20): Rₜ=1.62 min. MS: m/z=1112 (M+H)⁺

### Example 79A

### Benzyl 2(S)-(5-benzyloxycarbonylamino-2(S)-tert-butoxycarbonylamino-pentanoylamino)-3-[4,4′-bisbenzyloxy-3′-(2(S)-benzyloxycarbonylamino-2-pentafluorophenyloxycarbonylethyl)biphenyl-3-yl]propionate

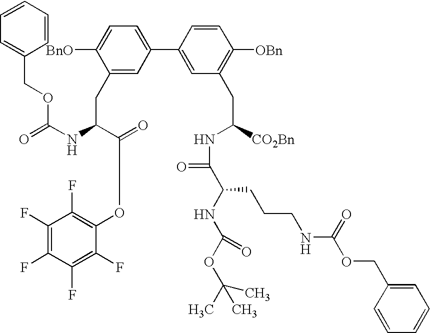

Preparation takes place in analogy to Example 18A (Method A) from 422 mg (0.38 mmol) of the compound from Example 78A and 349 mg (1.9 mmol) of pentafluorophenol with 80 mg (0.42 mmol) of EDCI and 4.63 mg (0.04 mmol) of DMAP in 4 ml of dichloromethane.

Yield: 502 mg (95% of theory). LC-MS (Method 20): Rₜ=3.13 min. MS: m/z=1278 (M+H)⁺

### Example 80A

### Benzyl 2(S)-(5-benzyloxycarbonylamino-2(S)-aminopentanoylamino)-3-[4,4′-bisbenzyloxy-3′-(2-(S)-benzyloxycarbonylamino-2-pentafluorophenyloxycarbonylethyl)biphenyl-3-yl]propionate hydrochloride

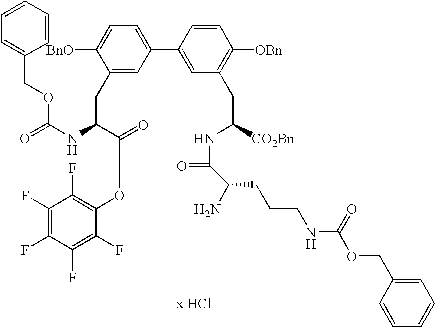

5 ml of 4 M dioxane/hydrogen chloride solution are added to 215 mg (0.17 mmol) of the compound from Example 79A while stirring in an ice bath. The mixture is stirred for one hour and evaporated to constant weight in vacuo.

Yield: 200 mg (92% of theory). LC-MS (Method 20): Rₜ=4.25 min. MS: m/z=1178 (M+H)⁺

### Example 81A

### Benzyl 5,17-bisbenzyloxy-14(S)-benzyloxycarbonylamino-11(S)-(3-benzyloxycarbonylaminopropyl)-10,13-dioxo-9,12-diazatricyclo[14.3.1.1^{2,6}]-henicosa-1(19),2,4,6(21),16(20),17-hexaene-8(S)-carboxylate

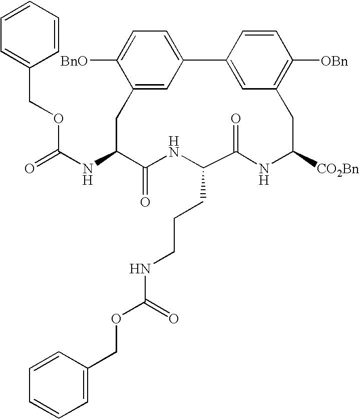

1.35 g (0.91 mmol) of the compound from Example 80A are introduced into 3 l of chloroform and, while stirring vigorously, 2.54 ml (18.2 mmol) of triethylamine in 50 ml of chloroform are added over the course of 20 min at RT. The mixture is stirred overnight and evaporated to dryness in vacuo. The residue is stirred with 5 ml of acetonitrile, filtered and dried to constant weight of the residue.

Yield: 890 mg (93% of theory). LC-MS (Method 20): Rₜ=5.10 min. MS: m/z=994 (M+H)⁺

### Example 82A

### (8S,11S,14S)-14-Amino-11-(3-aminopropyl)-5,17-dihydroxy-10,13-dioxo-9,12-diazatricyclo[14.3.1.1^{2,6}]-henicosa-1(20),2(21),3,5,6,18-hexaene-8-carboxylic acid dihydrochloride

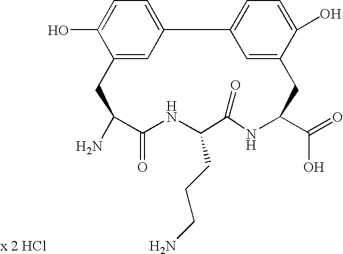

50 mg (0.05 mmol) of the compound from Example 81A are suspended in 50 ml of glacial acetic acid/water/ethanol (4/1/1), mixed with 30 mg of Pd/C (10%) catalyst and hydrogenated at RT for 20 hours. After the catalyst has been removed by filtration through kieselguhr, the filtrate is evaporated to dryness in vacuo and, while stirring, 2.5 ml of 0.1 N hydrochloric acid are added. The mixture is evaporated to dryness in vacuo and dried to constant weight.

Yield: 17 mg (63% of theory). TLC (methanol/dichloromethane/25% strength ammonia=5/3/2): R_{f}=0.6 LC-MS (Method 9): Rₜ=0.28 min. MS: m/z=457 (M+H)⁺

### Example 83A

### (8S,11S,14S)-14-[(tert-Butoxycarbonyl)amino-11-[3-[(tert-butoxycarbonyl)-amino]propyl}-5,17-dihydroxy-10,13-dioxo-9,12-diazatricyclo[14.3.1.1^{2,6}]-henicosa-1(20),2(21),3,5,16,18-hexaene-8-carboxylic acid

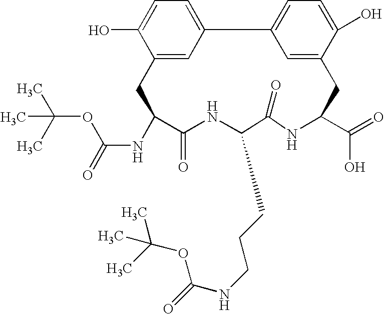

225 mg (0.42 mmol) of the compound from Example 82A are dissolved in 2.25 ml of water and 2.25 ml of 1 N sodium hydroxide solution, cooled in an ice bath and, while stirring, 278 mg (1.27 mmol) of di-tert-butyl dicarbonate are added. The temperature is raised briefly after the addition to 30° C., and reaction is allowed to continue at RT overnight. The mixture is acidified to about pH=5 with 0.1 N hydrochloric acid and cautiously evaporated to dryness in vacuo at RT. The residue is stirred with diethyl ether, filtered and dried to constant weight thereof.

Yield: 259 mg (93% of theory). LC-MS (Method 18): Rₜ=1.96 min. MS: m/z=656 (M+H)⁺

### Example 84A

### 2-(Trimethylsilyl)ethyl 2-(benzyloxy)-N-[(benzyloxy)carbonyl]-4-(4,4,5,5-tetramethyl-1,3,2-dioxaborolan-2-yl)-L-phenylalaninate

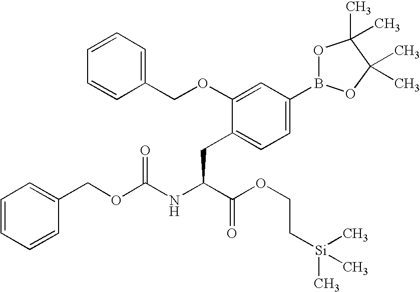

0.924 g (3.64 mmol, 1.15 eq) of 4,4,4′,4′,5,5,5′,5′-octamethyl-2,2′-bi-1,3,2-dioxaborolane, 0.932 g (9.50 mmol, 3 eq) of potassium acetate and 0.116 g (0.160 mmol, 0.05 eq) of bis(diphenylphosphino)ferrocenepalladium(II) chloride are added at RT to a degassed solution of 2.00 g (3.17 mmol) of (2-trimethylsilyl)ethyl 2(S)-benzyloxycarbonylamino-3-(2-benzyloxy-5-iodophenyl)propionate (Example 11A) in 20 ml of DMF. The mixture is stirred at 80° C. for 6 hours. It is taken up in water and ethyl acetate, the phases are separated, and the aqueous phase is washed several times with ethyl acetate. The combined organic phases are dried over sodium sulfate and concentrated in vacuo. The crude product is purified by chromatography on silica gel (cyclohexane/ethyl acetate 10: 1).

Yield: 1.12 g (56% of theory). LC-MS (Method 22): Rₜ=4.50 min. MS (EI): m/z=632 (M+H)⁺¹H-NMR (200 MHz, CDCl₃): δ=0.92 (dd, 2H), 1.31 (s, 12H), 2.95-3.95 (m, 2H), 4.11 (m_{c}, 2H), 4.55 (11 (m_{c}, 1H), 4.99 (s, 2H), 5.08 (s, 2H), 5.53 (d, 1H), 6.90 (d, 1H), 7.15-7.47 (m, 10H), 7.58 (d, 1H), 7.67 (dd, 1H).

Examples 85A to 87A listed in the following table can be prepared in analogy to Example 37A.

| Example | | Analytical |
|---|---|---|
| No. | Structure | data |
| 85A | 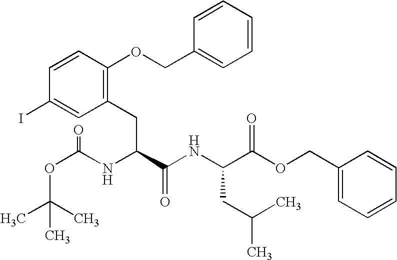 | LC-MS (Method 15): Rₜ = 3.12 min. MS (EI): m/z = 701 (M + H)⁺ |
| 86A | 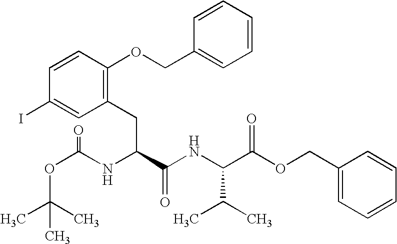 | LC-MS (Method 15): Rₜ = 3.08 min. MS (EI): m/z = 687 (M + H)⁺ |
| 87A | 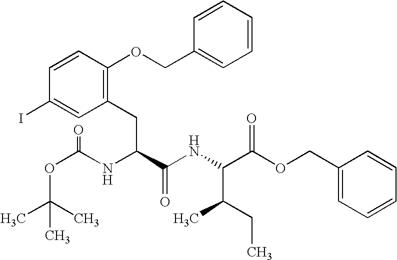 | LC-MS (Method 15): Rₜ = 3.14 min. MS (EI): m/z = 701 (M + H)⁺ |

Examples 88A to 90A listed in the following table can be prepared in analogy to Example 42A.

| Example | | Analytical |
|---|---|---|
| No. | Structure | data |
| 88A | 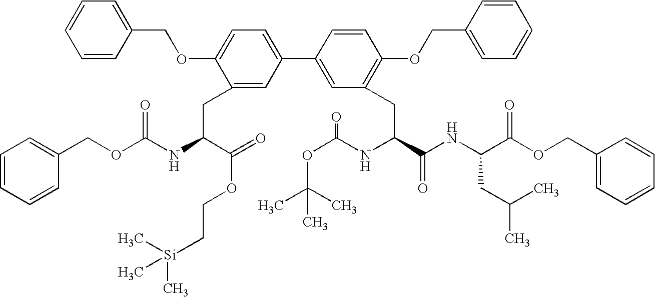 | LC-MS (Method 16): Rₜ = 2.59 min. MS (EI): m/z = 1078 (M + H)⁺ |
| 89A | 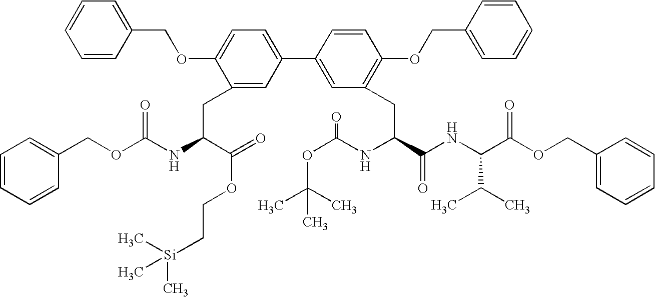 | LC-MS (Method 15): Rₜ = 3.49 min. MS (EI): m/z = 1064 (M + H)⁺ |
| 90A | 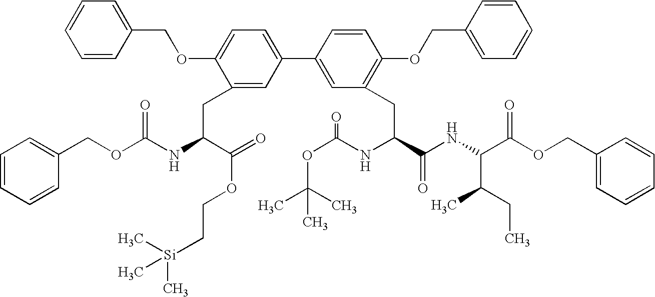 | LC-MS (Method 15): Rₜ = 3.55 min. MS (EI): m/z = 1078 (M + H)⁺ |

Examples 91A to 93A listed in the following table can be prepared in analogy to Example 47A.

| Example | | Analytical |
|---|---|---|
| No. | Structure | data |
| 91A | 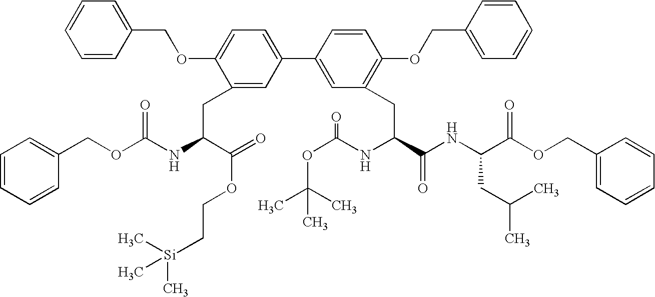 | LC-MS (Method 16): Rₜ = 2.59 min. MS (EI): m/z = 1078 (M + H)⁺ |
| 92A | 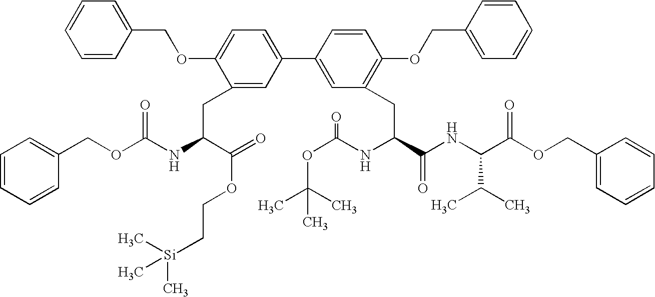 | — |
| 93A | 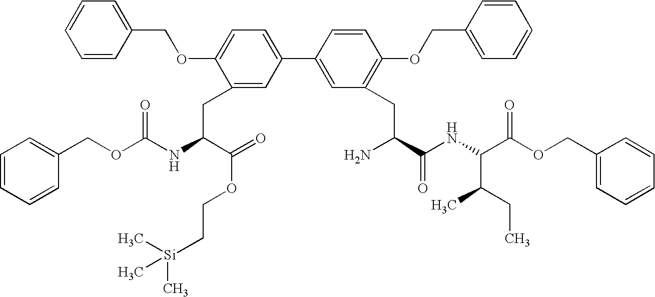 | — |

Example 94A to 96A listed in the following table can be prepared in analogy to Example 52A.

| Example | | |
|---|---|---|
| No. | Structure | Analytical data |
| 94A | 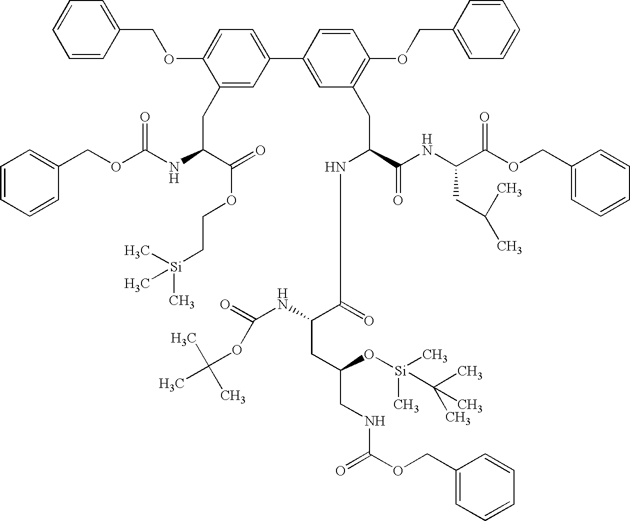 | LC-MS (Method 16): Rₜ = 3.40 min. MS (EI): m/z = 1457 (M+H)⁺ |
| 95A | 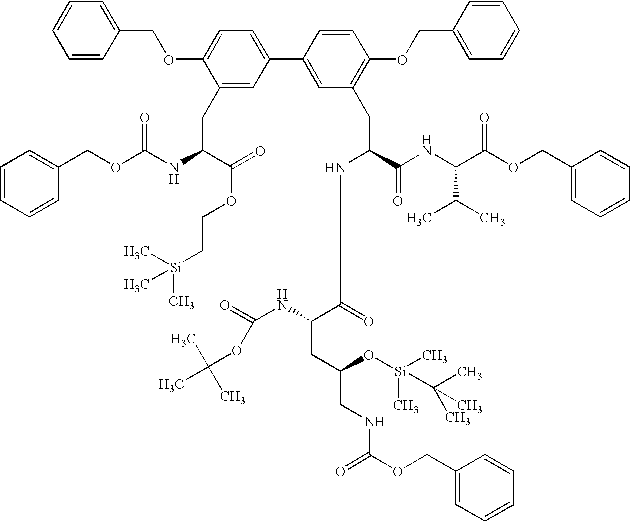 | LC-MS (Method 16): Rₜ = 3.17 min MS (EI): m/z = 1442 (M+H)⁺ |
| 96A | 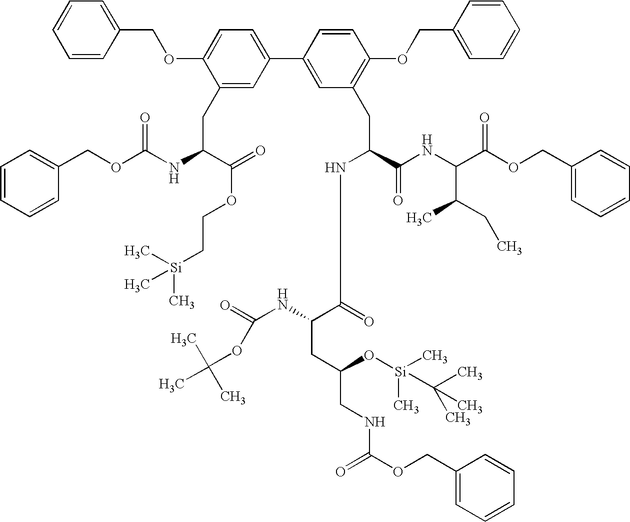 | LC-MS (Method 16): Rₜ = 3.33 min MS (EI): m/z = 1457 (M+H)⁺ |

Examples 97A to 99A listed in the following table can be prepared in analogy to Example 57A.

| Example | |
|---|---|
| No. | Structure |
| 97A | 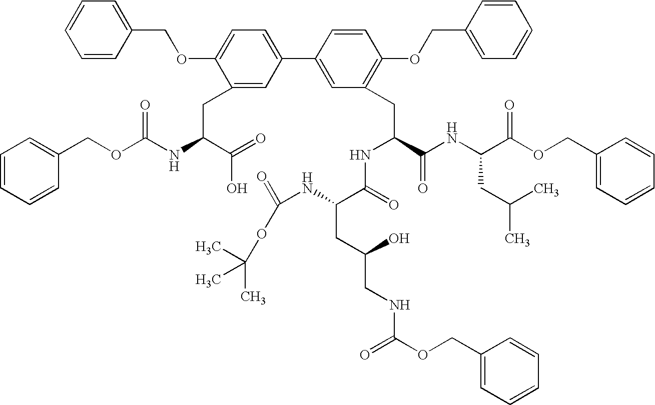 |
| 98A | 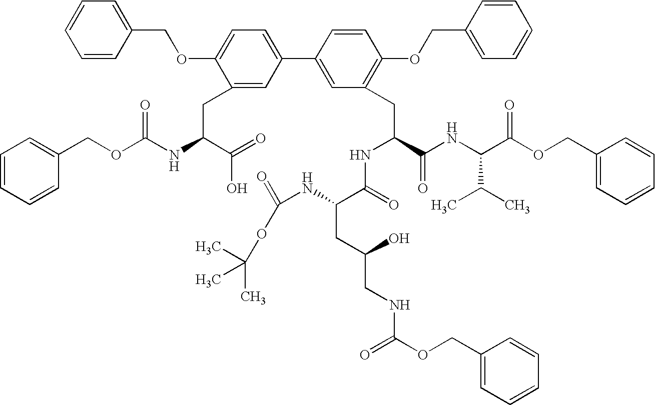 |
| 99A | 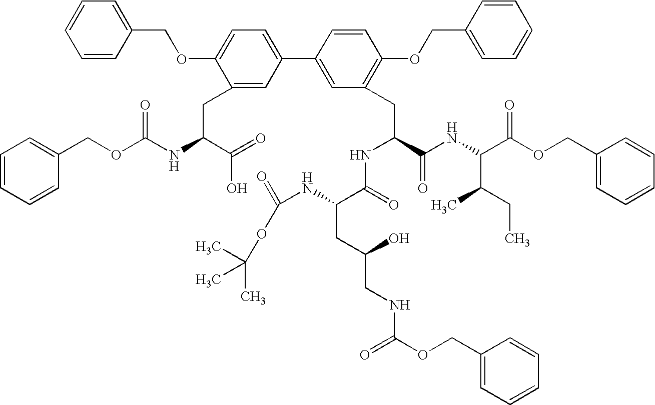 |

Examples 100A to 102A listed in the following table can be prepared in analogy to Example 62A.

| Example | |
|---|---|
| No. | Structure |
| 100A | 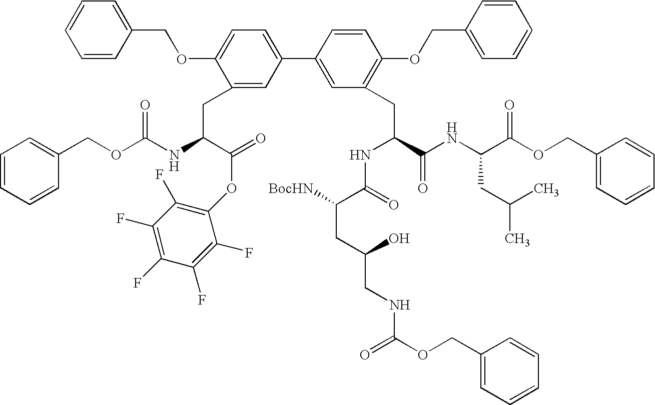 |
| 101A | 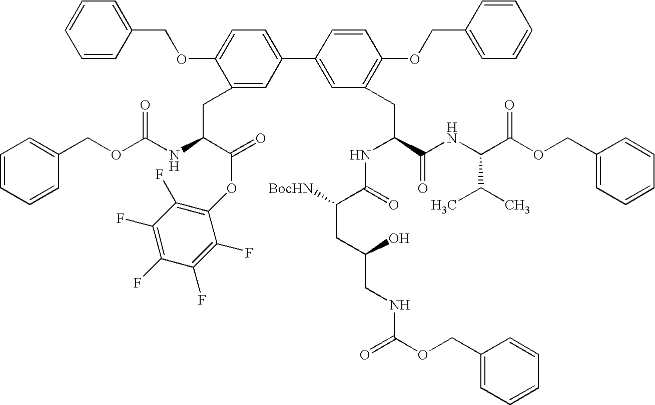 |
| 102A | 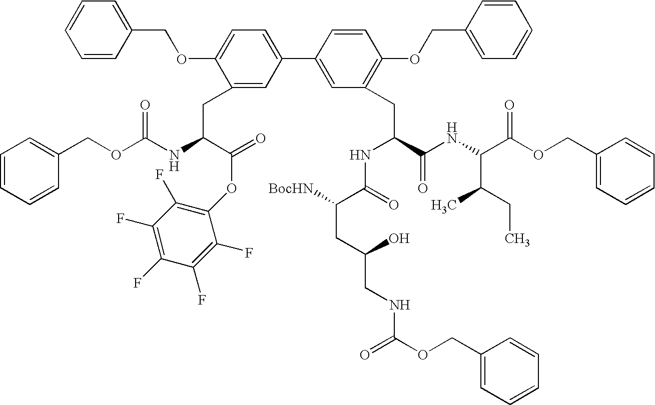 |

Examples 103A to 105A listed in the following table can be prepared in analogy to Example 67A.

| Example | |
|---|---|
| No. | Structure |
| 103A | 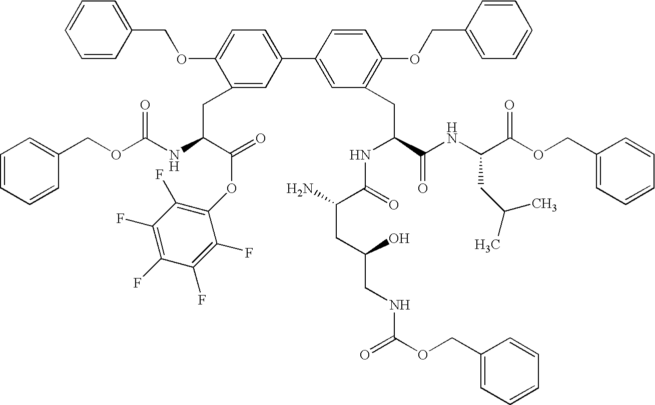 |
| 104A | 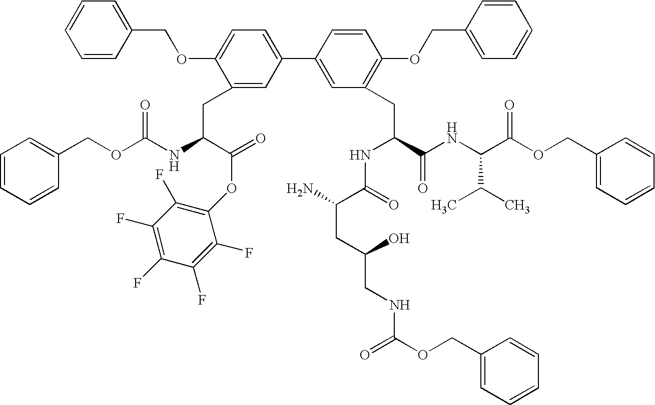 |
| 105A | 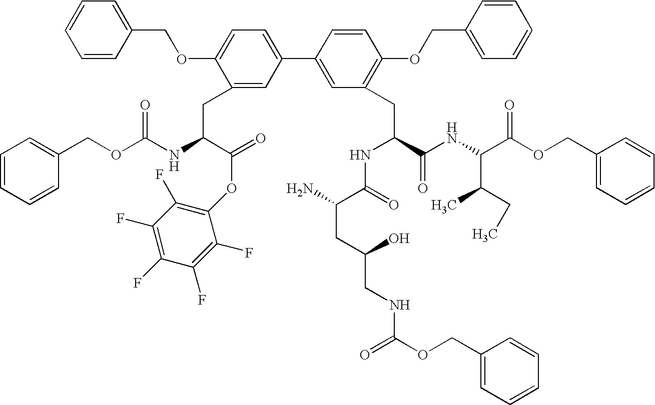 |

Examples 106A to 108A listed in the following table can be prepared in analogy to Example 72A.

| Example | | Analytical |
|---|---|---|
| No. | Structure | data |
| 106A | 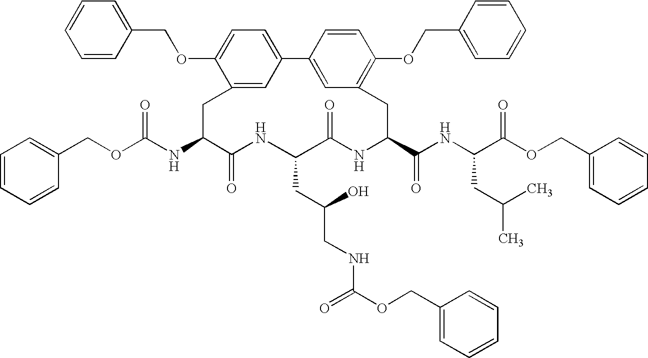 | LC-MS (Method 15): Rₜ = 3.10 min. MS (EI): m/z = 1124 (M + H)⁺ |
| 107A | 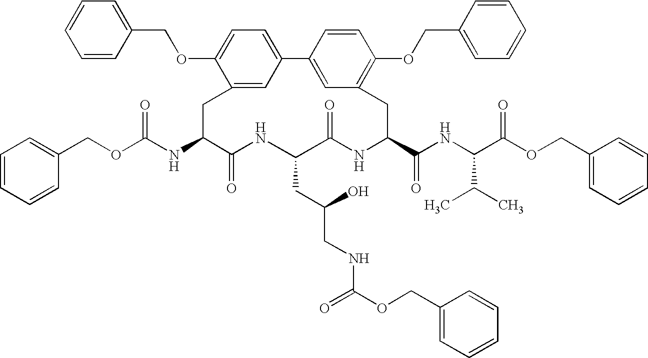 | LC-MS (Method 24): Rₜ = 3.31 min. MS (EI): m/z = 1110 (M + H)⁺ |
| 108A | 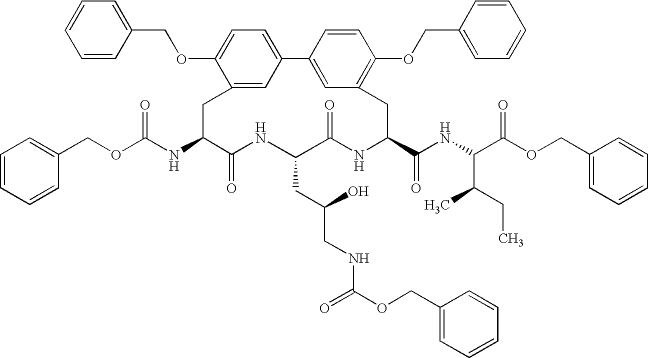 | LC-MS (Method 24): Rₜ = 3.32 min. MS (EI): m/z = 1124 (M + H)⁺ |

Example 109A detailed in the following table can be prepared in analogy to Example 24A.

| Example | | Analytical |
|---|---|---|
| No. | Structure | data |
| 109A | 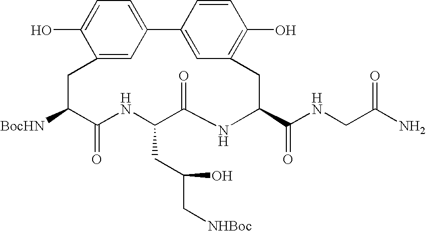 | LC-MS (Method 24): Rₜ = 1.94 min. MS (EI): m/z = 729 (M + H)⁺ |

### Example 110A

### 2-(Benzyloxy)-N-(tert-butoxycarbonyl)iodo-N-methyl-L-phenylalanine

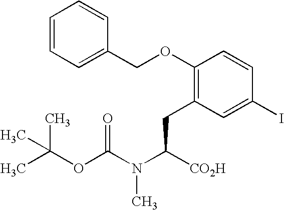

Under an argon atmosphere, 500 mg (1 mmol) of the compound from Example 6A are dissolved in 20 ml of THF, 90.5 mg (3.02 mmol) of sodium hydride and 0.51 ml (1141.6 mg; 8.04 mmol) of methyl iodide (80% pure) are added, and the mixture is stirred at room temperature overnight. It is diluted with 25 ml of ethyl acetate and 25 ml of water and adjusted to pH=9 with 0.1 N hydrochloric acid. The mixture is concentrated to a small volume in vacuo. 10 ml of ethyl acetate and 10 ml of water are added, the mixture is shaken vigorously, and the organic phase is separated off. Drying with sodium sulfate and concentration in vacuo result in 140 mg of product (19% of theory).

The aqueous phase is acidified (pH=3) and extracted three times with 20 ml of ethyl acetate. Concentration in vacuo and drying in vacuo result in 351 mg of product (68% of theory).

LC-MS (Method 17): Rₜ=3.9 min. MS (EI): m/z=511 (M+H)⁺

### Example 111A

### Benzyl 2-(benzyloxy)-N-(tert-butoxycarbonyl)-5-iodo-N-methyl-L-phenylalaninate

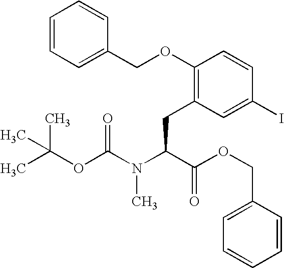

Preparation takes place in analogy to Example 7A from 350 mg (0.68 mmol) of the compound from Example 110A, 8.29 mg (0.07 mmol) of DMAP, 148 mg (1.37 mmol) of benzyl alcohol and 157.46 mg (0.82 mmol) of EDC in 3 ml of acetonitrile.

Yield: 382 mg (93% of theory). LC-MS (Method 17): Rₜ=4.8 min. MS (EI): m/z=601 (M+H)⁺

### Example 112A

### Benzyl 2-(benzyloxy)-N-(tert-butoxycarbonyl)-N-methyl-5-(4,4,5,5-tetramethyl-1,3,2-dioxaborolan-2-yl)-L-phenylalaninate

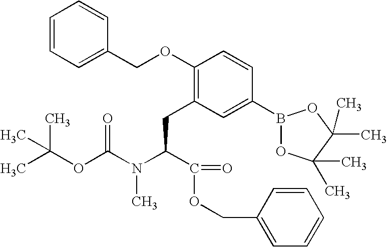

In analogy to Example 8A, 380 mg (0.63 mmol) of the compound from Example 111A are. introduced into 4 ml of DMF in a heat-dried flask and, while stirring at room temperature, 184.5 mg (0.73 mmol) of 4,4,4′,4′,5,5,5′,5′-octamethyl-2,2′-bi-1,3,2-dioxaborolane, 186 mg (1.9 mmol) of potassium acetate and 23.15 mg (0.03 mmol) of bis(diphenylphosphino)ferrocenepalladium(II) chloride are added. Reaction is allowed to take place at 80° C. for 4 h. The product is obtained after workup and chromatography (silica gel 60, mobile phase: cyclohexane/ethyl acetate=4/1).

Yield: 196 mg LC-MS (Method 17): Rₜ=4.9 min. MS (EI): m/z=601 (M+H)⁺

### Example 113A

### 2-(Trimethylsilyl)ethyl 2(S)-benzyloxycarbonylamino-3-[4,4′-bisbenzyloxy-3′-(2(S)-benzyloxycarbonyl-(2-tert-butoxycarbonyl-2-methyl)aminoethyl)biphenyl-3-yl]propionate

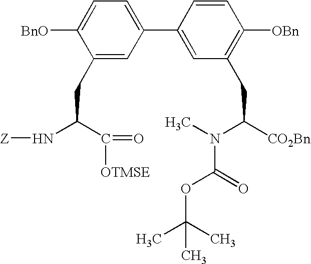

Preparation takes place in analogy to Example 12A (Method B) from 190 mg (0.32 mmol) of the compound from Example 112A, 199.5 mg (0.32 mmol) of the compound from Example 11A, 195.5 mg (0.63 mmol) of cesium carbonate and 23.15 mg (0.03 mmol) of bis(diphenylphosphino)ferrocenepalladium(II) chloride in 1.5 ml of DMF under an argon atmosphere.

Yield: 212 mg (66% of theory). LC-MS (Method 25): Rₜ=4.86 min. MS (EI): m/z=978 (M+H)⁺

### Example 114A

### 2-(Trimethylsilyl)ethyl 2(S)-benzyloxycarbonylamino-3-[4,4′-bisbenzyloxy-3′-(2(S)-benzyloxycarbonyl-2-methylaminoethylbiphenyl-3-yl]propionate hydrochloride

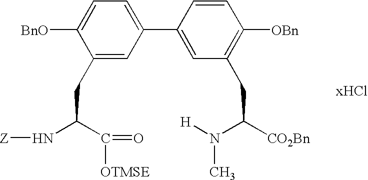

Preparation takes place in analogy to Example 15A from 930 mg (0.95 mmol) of the compound from Example 113A and 22.14 ml of a 4 M dioxane/hydrogen chloride solution in 15 ml of dioxane.

Yield: 915 mg (78% of theory). LC-MS (Method 25): Rₜ=2.53 min. MS (EI): m/z=878 (M+H)⁺

### Example 115A

### Benzyl 2(S)-{methyl-[5-benzyloxycarbonylamino-2(S)-tert-butoxycarbonylamino-4(R)-(tert-butyldimethylsilyloxy)pentanoyl]amino}-3-{4,4′-bisbenzyloxy-3′-[2(S)-benzyloxycarbonylamino-2-(2-trimethylsilylethoxycarbonyl)ethyl]-biphenyl-3-yl}propionate

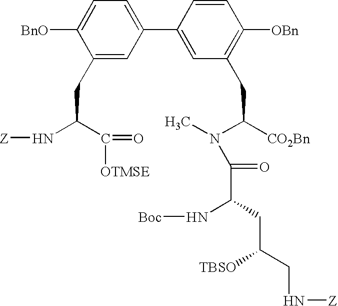

Preparation takes place in analogy to Example 16A from 922 mg (1.01 mmol) of the compound from Example 114A, 0.5 g (1.01 mmol) of the compound from Example 14A, 421 mg (1.11 mmol) of HATU and 0.7 ml (518 mg; 3.27 mmol) of DIPEA in 4.2 ml of DMF.

Yield: 703 mg (51% of theory). LC-MS (Method 16): Rₜ=3.17 min. MS (EI): m/z=1356 (M+H)⁺

### Example 116A

### 2(S)-Benzyloxycarbonylamino-3-{4,4′-bisbenzyloxy-3′-[2(S)-benzyloxycarbonyl-2-{methyl-(5-benzyloxycarbonylamino-2(S)-tert-butoxycarbonylamino-4(R)-hydroxypentanoyl)amino}ethyl]biphenyl-3-yl}propionic acid

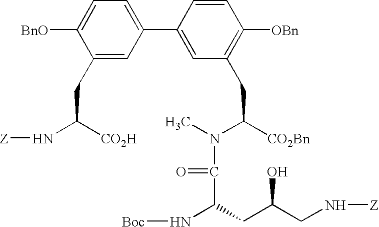

Preparation takes place in analogy to Example 17A from 360 mg (0.27 mmol) of the compound from Example 115A and 0.8 ml (3 eq) of 1 M tetrabutylammonium fluoride solution (THF) in 20 ml of DMF.

Yield: 159 mg (53% of theory). LC-MS (Method 23): Rₜ=3.19 min. MS (EI): m/z=1142 (M+H)⁺

### Example 117A

### Benzyl 2(S)-[methyl-(5-benzyloxycarbonylamino)-2(S)-tert-butoxycarbonylamino-4(R)-hydroxypentanoyl]amino-3-[4,4′-bisbenzyloxy-3′-(2(S)-benzyloxycarbonylamino-2-pentafluorophenyloxycarbonylethyl)biphenyl-3-yl]propionate

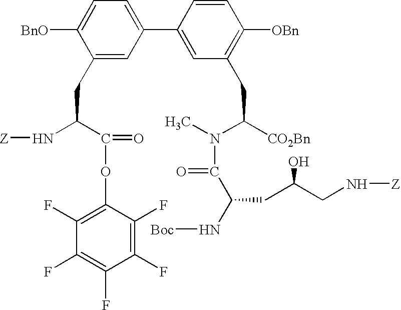

Preparation takes place in analogy to Example 18A (Method A) from 330 mg (0.29 mmol) of the compound from Example 116A, 265.6 mg (1.44 mmol) of pentafluorophenol, 3.53 mg (0.03 mmol) of DMAP and 60.87 mg (0.32 mmol) of EDC in 10 ml of dichloromethane.

Yield: 271 mg (69% of theory). LC-MS (Method 23): Rₜ=3.38 min. MS (EI): m/z=1308 (M+H)⁺

### Example 118A

### Benzyl 2(S)-[methyl-(5-benzyloxycarbonylamino)-2(S)-amino-4(R)-hydroxypentanoyl]amino-3-[4,4′-bisbenzyloxy-3′-(2(S)-benzyloxycarbonylamino-2-pentafluorophenyloxycarbonylethyl)biphenyl-3-yl]propionate hydrochloride

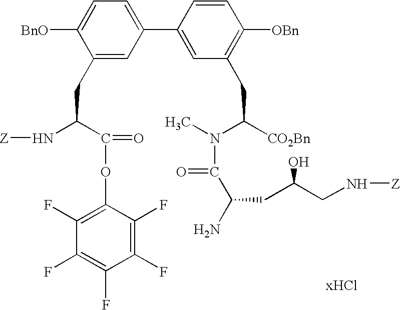

130 mg (0.1 mmol) of the compound from Example 117A are dissolved in 0.5 ml of dioxane, and 5 ml of 4 M dioxane/hydrogen chloride solution are cautiously added (ice bath). After 30 minutes, reaction is allowed to continue at room temperature for a further 2 h. The mixture is evaporated to dryness in vacuo and dried to constant weight under high vacuum.

Yield: 130 mg (70% of theory). LC-MS (Method 15): Rₜ=2.68 min. MS (EI): m/z=1208 (M+H)⁺

### Example 119A

### Benzyl (8S,11S,14S)-5,17-bis(benzyloxy)-14-{[(benzyloxy)carbonyl]amino}-11-((2R)-3-{[(benzyloxy)carbonyl]amino}-2-hydroxypropyl-9-methyl-10,13-dioxo-9,12-diazatricyclo[14.3.1.1^{2,6}]henicosa-1(20),2(21),3,5,16,18-hexaene-8-carboxylate

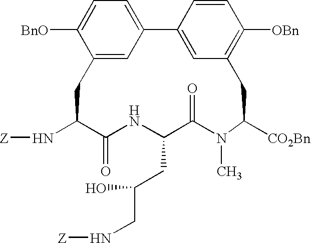

130 mg (0.1 mmol) of the compound from Example 118A are introduced into 220 ml of dry chloroform. While stirring at room temperature, 23 ml (20 eq) of triethylamine in 5 ml of dichloromethane are added over the course of 20 minutes. The mixture is stirred overnight. It is then evaporated to dryness in vacuo. The residue is stirred with acetonitrile. Drying of the residue results in 44 mg of product. Further product (30 mg) is obtained from the mother liquor by RP-HPLC.

Yield: 74 mg (69% of theory). LC-MS (Method 15): Rₜ=3.13 min. MS (EI): m/z=1024 (M+H)⁺

### Example 120A

### (8S,11S,14S)-14-Amino-11-[(2R)-3-amino-2-hydroxypropyl]-5,17-dihydroxy-9-methyl-10,13-dioxo-9,12-diazatricyclo[14.3.1.1^{2,6}]henicosa-1(20),2(21),3,5,16,18-hexaenecarboxylic acid ditrifluoroacetate

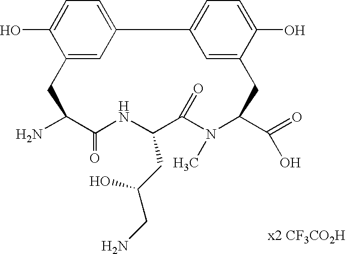

33 mg (0.032 mmol) of the compound from Example 119A are cautiously treated with dilute trifluoroacetic acid. The resulting clear solution is then lyophilized.

Yield: 23 mg (quantitative) LC-MS (Method 15): Rₜ=0.92 min. MS (EI): m/z=486 (M+H)⁺

### Example 121A

### (8S,11S,14S)-5,17-Bis(benzyloxy)-14-{[benzyloxycarbonyl]amino}-11-(2R)-3-{[benzyloxycarbonyl]amino}-2-hydroxypropyl-9-methyl-10,13-dioxo-9,12-diazatricyclo[14.3.1.1^{2,6}]henicosa-1(20),2(21),3,5,16,18-hexaene-8-carboxylic acid

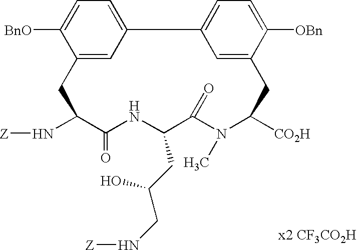

37 mg (0.04 mmol) of the compound from Example 119A are dissolved in 2 ml of THF, 0.14 ml of 1 N lithium hydroxide solution is added, and the mixture is stirred at room temperature for 3 h. It is then acidified with 1 N hydrochloric acid and evaporated to dryness under high vacuum.

Yield: 33 mg (71% of theory). LC-MS (Method 23): Rₜ=2.90 min. MS (EI): m/z=934 (M+H)⁺

### Example 122A

### (8S,11S,14S)-5,17-Bis(benzyloxy)-14-{[benzyloxycarbonyl]amino}-11-(2R)-3-{[benzyloxycarbonyl]amino}-2-hydroxypropyl-9-methyl-10,13-dioxo-9,12-diazatricyclo[14.3. 1.1^{2,6}]henicosa-1(20),2(21),3,5,16,18-hexaene-8-carboxamide

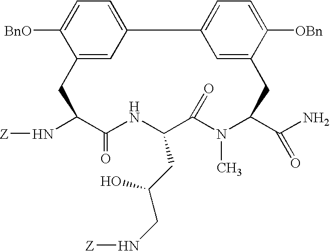

30 mg (0.03 mmol) of the compound from Example 121A are dissolved in 1 ml of DMF, and 0.01 ml (3 eq) of triethylamine is added. After the reaction solution has been cooled in an ice bath, 8.76 mg (2 eq) of isobutyl chloroformate are added, and the reaction is allowed to take place for 30 minutes. After stirring at room temperature for a further hour, 0.64 ml (10 eq) of 0.5 N dioxane/ammonia solution is added, and the mixture is stirred overnight. The residue after concentration in vacuo is purified by RP-HPLC.

Yield: 11 mg (37% of theory). LC-MS (Method 23): Rₜ=2.91 min. MS (EI): m/z=934 (M+H)⁺

Examples 123A to 129A listed in the following table are prepared from the appropriate precursors in analogy to the methods detailed above for Examples 115A to 122A:

| | | Preparation | |
|---|---|---|---|
| Example | | analogous | Analytical |
| No. | Structure | to | data |
| 123A | 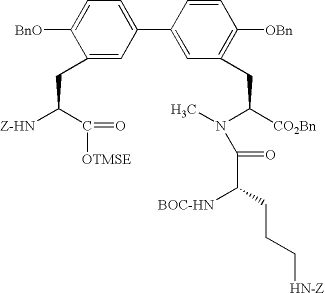 | 115A | LC-MS (Method 25): Rₜ = 4.85 min. MS (EI): m/z = 1226 (M + H)⁺ |
| 124A | 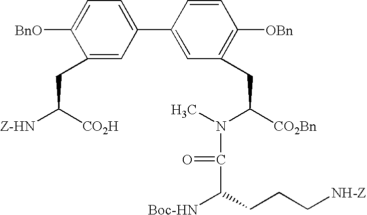 | 116A | LC-MS (Method 25): Rₜ = 2.04 min. MS (EI): m/z = 1126 (M + H)⁺ |
| 125A | 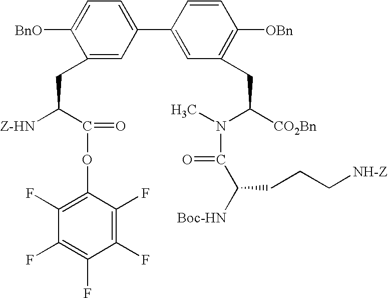 | 117A | LC-MS (Method 25): Rₜ = 3.79 min. MS (EI): m/z = 1292 (M + H)⁺ |
| 126A | 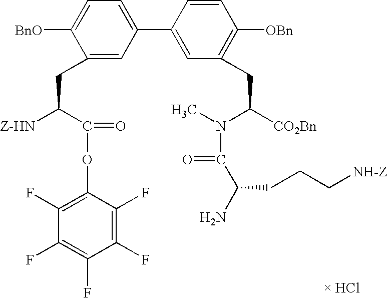 | 118A | LC-MS (Method 25): Rₜ = 3.72 min. MS (EI): m/z = 1192 (M + H)⁺ |
| 127A | 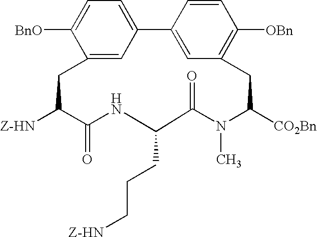 | 119A | LC-MS (Method 25): Rₜ = 4.39 min. MS (EI): m/z = 1008 (M + H)⁺ |
| 128A | 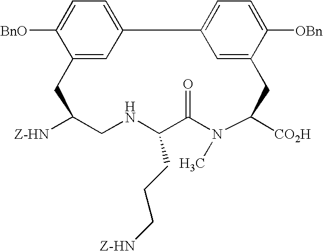 | 121A | LC-MS (Method 26): Rₜ = 3.64 min. MS (EI): m/z = 918 (M + H)⁺ |
| 129A | 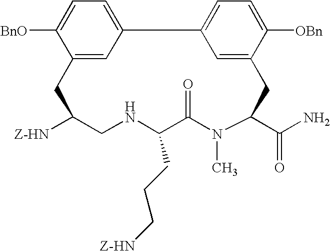 | 122A | LC-MS (Method 25): Rₜ = 3.8 min. MS (EI): m/z = 917 (M + H)⁺ |

### Example 130A

### Benzyl 2(S)-tert-butoxycarbonylamino-5-nitro-4-oxopentanoate

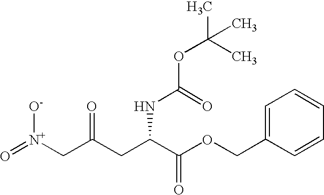

A solution A of 10 g (30.9 mmol) of 2(S)-tert-butoxycarbonylaminosuccinic acid 1-benzyl ester and 5.27 g (32.5 mmol) of 1,1′-carbonyldiimidazole in 100 ml of tetrahydrofuran is stirred at RT for 5 h. 18.8 g (30.9 mmol) of nitromethane are added dropwise to a solution B of 3.2 g (34.2 mmol) of potassium tert-butoxide in 100 ml of tetrahydrofuran at 0° C. Solution B is stirred while warming to RT, and then solution A is added dropwise at RT. The resulting mixture is stirred at RT for 16 h and adjusted to pH 2 with 20% strength hydrochloric acid. The solvent is evaporated. The remaining crude product is taken up in ethyl acetate/water. After separation of the phases, the organic phase is extracted twice with water, dried over sodium sulfate and concentrated. 13 g (99% of theory) of the product are obtained.

MS (ESI): m/z=334 (M+H)⁺¹H-NMR (300 MHz, d₆-DMSO): δ=1.37 (s, 9H), 2.91 (m, 1H), 3.13 (m, 1H), 4.44 (m, 1H), 5.12 (s, 2H), 5.81 (m, 2H), 7.2-7.5 (m, 5H).

### Example 131A

### Benzyl 2(S)-tert-butoxycarbonylamino-4(R)-hydroxy-5-nitropentanoate

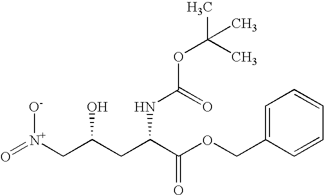

A solution of 11.3 g (30.8 mmol) of benzyl 2(S)-tert-butoxycarbonylamino-5-nitro-4-oxopentanoate in 300 ml of tetrahydrofuran is cooled to −78° C., 30.8 ml of a 1M solution of L-Selectrid® in tetrahydrofuran are added dropwise, and the mixture is stirred at −78° C. for 1 h. After warming to RT, saturated ammonium chloride solution is cautiously added to the solution. The reaction solution is concentrated, and the residue is taken up in water and ethyl acetate. The aqueous phase is extracted three times with ethyl acetate. The combined organic phases are dried over sodium sulfate and evaporated. The crude product is prepurified on silica gel 60 (mobile phase: cyclohexane/ethyl acetate 10/1), and the collected fractions are concentrated and stirred with cyclohexane/ethyl acetate 5/1. The remaining crystals are filtered off with suction and dried. 2.34 g (21% of theory) of the desired diastereomer are obtained. Chromatographic separation of the mother liquor on Lichrospher Diol 10 μM (mobile phase: ethanol/iso-hexane 5/95) results in a further 0.8 g (6.7%) of the product.

MS (ESI): m/z=369 (M+H)⁺¹H-NMR (300 MHz, d₆-DMSO): δ=1.38 (s, 9H), 1.77 (m, 1H), 1.97 (m, 1H), 4.10-4.44 (m, 3H), 4.67 (m, 1H), 5.12 (m, 2H), 5.49 (d, 1H), 7.25-7.45 (m, 5H).

### EXEMPLARY EMBODIMENTS

The synthesis of exemplary embodiments can start from partially protected biphenomycin derivatives (such as, for example, 21A).

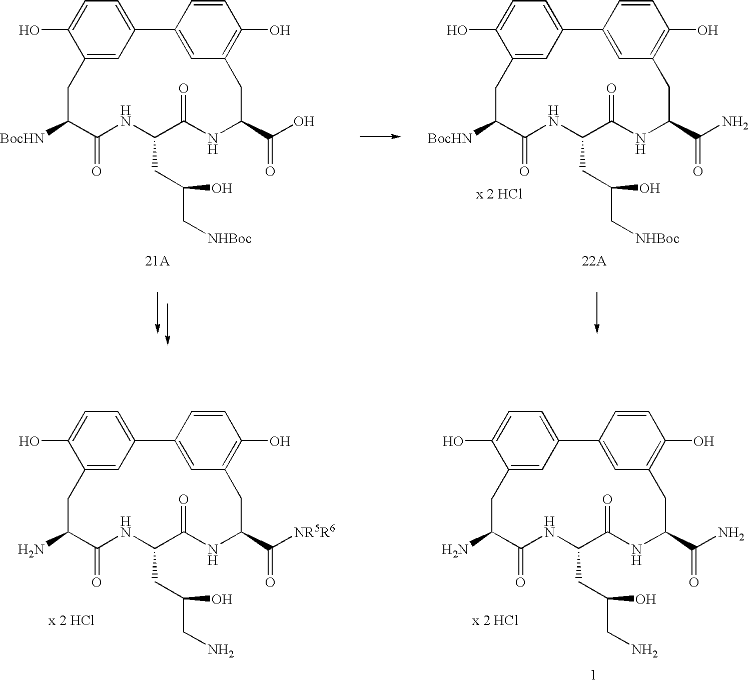

### Example 1

### (8S,11S,14S)-14-Amino-11-[(2R)-3-amino-2-hydroxypropyl]-5,17-dihydroxy-10,13-dioxo-9,12-diazatricyclo[14.3.1.1^{2,6}]henicosa-1(20),2(21),3,5,16,18-hexaene-8-carboxamide dihydrochloride

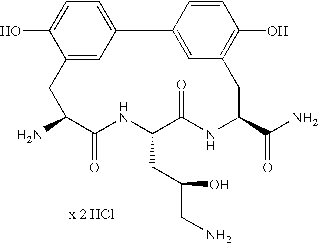 Method A:

A 4 M solution of hydrochloric acid gas in dioxane (1.0 ml) is added dropwise to a solution of 2.15 mg (3.2 μmol) of tert-butyl(2R)-3-[(8S,11S,14S)-8-(aminocarbonyl)-14-[(tert-butoxycarbonyl)amino]-5,17-dihydroxy-10,13-dioxo-9,12-diazatricyclo[14.3.1.1^{2,6}]henicosa-1(20),2(21),3,5,16,18-hexaene-11-yl]-2-hydroxypropylcarbamate (Example 22A) in dry dioxane (analytical grade, 1.0 ml) under argon. Complete conversion is reached after about 30 min. The reaction mixture is frozen and freeze dried to remove solvents. Purification takes place by gel chromatography [Sephadex LH-20; methanol/concentrated hydrochloric acid (1:0.0001) doped with sodium disulfite], resulting in 1.4 mg (80% of theory) of product.

HPLC-UV-Vis (Method 14): Rₜ=3.09 min. λₘₐₓ (qualitative)=˜204 nm (s), 269 (m), ˜285 (sh) (H₂O/acetonitrile+0.01% TFA [7:3]). ¹H-NMR (500 MHz, CD₃OD): δ=1.79 (ddd, 1H, J=13.6, 9.2, 5.9 Hz), 1.99 (ddd, 1H, J=13.6, 9.6, 4.0 Hz), 2.82 (dd, 1H, J=12.8, 9.6 Hz), 2.87 (dd, 1H, J=17.1, 12.1 Hz), 3.04 (dd, 1H, J=12.8, 2.9 Hz), 3.11 (dd, 1H, J=14.8, 3.0 Hz), 3.38 (dd, 1H, J=16.9, 1.9 Hz), 3.57 (dd, 1H, J=11.7, 5.4 Hz), 3.92 (tt, 1H, J=9.4, 3.5 Hz), 4.23 (dd, 1H, J=4.9, 3.0 Hz), 4.90 (m, 1H), 4.91 (m, 1H), 6.79 (d, 1H, J=8.3 Hz), 6.85 (d, 1H, J=8.4 Hz), 7.10 (d, 1H, J=2.3 Hz), 7.25 (dd, 1H, J=8.3, 2.3 Hz), 7.36 (dd, 1H, J=8.5, 2.4 Hz), 7.44 (d, 1H, J=2.1 Hz). ¹³C NMR (125.5 MHz, CD₃OD): δ=30.3, 30.8, 39.5, 45.4, 50.6, 53.8, 55.3, 65.3, 115.6, 116.3, 120.8, 125.3, 126.2, 126.8, 127.0, 130.9, 132.7, 133.5, 155.0, 155.7, 168.4, 172.8, 177.0. LC-HR-FT-ICR-MS (Method 13): calc. for C₂₃H₃₀N₅O₆ [M+H]⁺ 472.2191 found 472.2191.

Method B:

Under argon, 14.8 mg (0.02 mmol) of tert-butyl (2R)-3-[(8S, 11S,14S)-8-(aminocarbonyl)-14-[(tert-butoxycarbonyl)amino]-5,17-dihydroxy-10,13-dioxo-9,12-diazatricyclo[14.3.1.1^{2,6}]henicosa-1(20),2(21),3,5,16,18-hexaene-11-yl]-2-hydroxypropylcarbamate (Example 22A) are introduced into 0.5 ml of dioxane. The mixture is cooled to 0° C., and 0.8 ml of 4 M hydrochloric acid solution in dioxane is 10 added dropwise. After 45 min, the mixture is concentrated in vacuo, and the residue is taken up twice more in dioxane and again concentrated in vacuo. The product is dried under high vacuum.

Yield: 12 mg (100% of theory). HPLC (Method 8): Rₜ=4.87 min. MS (EI): m/z=472 (M+H-2HCl)⁺. ¹H-NMR (400 MHz, D₂O): δ=0.58-0.67 (m, 2H), 1.65-1.86 (m, 3H), 1.88-1.98 (m, 1H), 2.03-2.13 (m, 1H), 2.87-3.02 (m, 4H), 3.09-3.19 (m, 2H), 3.38 (d, 1H), 3.59-3.69 (m, 2H), 3.88-3.96 (m, 1H), 4.46-4.51 (m, 1H), 4.85-5.01 (m, 5H), 6.98 (dd, 2H), 7.05 (dd, 1H), 7.36 (s, 1H), 7.43 (dd, 1H), 7.50 (dd, 1H).

### Example 2

### (8S,11S,14S)-14-Amino-11-[(2R)-3-amino-2-hydroxypropyl]-N-benzyl-5,17-dihydroxy-10,13-dioxo-9,12-diazatricyclo[14.3.1.1^{2,6}]henicosa-1(20),2(21),3,5,16,18-hexaene-8-carboxamide dihydrochloride

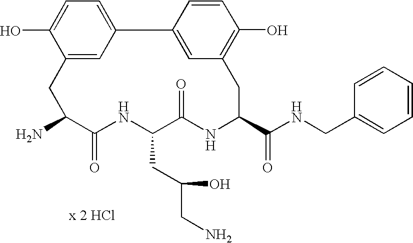

0.5 ml of 4 N hydrochloric acid solution in dioxane is added dropwise to a solution of tert-butyl (2R)-3-[(8S,11S,14S)-8-[(benzylamino)carbonyl]-14-[(tert-butoxycarbonyl)amino]-5,17-dihydroxy-10,13-dioxo-9,12-diazatricyclo[14.3.1.1^{2,6}]-henicosa-1(20),2(21),3,5,16,18-hexaene-11-yl]-2-hydroxypropylcarbamate (Example 23A) in 0.5 ml of 1,4-dioxane while cooling in ice. The ice cooling is removed and the mixture is stirred at RT for 2 h before being concentrated in vacuo and dried under high vacuum. The residue is taken up in a mixture of dichloromethane and methanol, and the solvents are evaporated off overnight.

LC-MS (Method 7): Rₜ=2.02 min. MS (ESI-pos): m/z=562 (M+H-2HCl)⁺. ¹H-NMR (400MHZ, D₂O): δ=1.70-1.81 (m, 1H), 1.82-1.91 (m, 1H), 2.71-2.84 (m, 2H1), 2.89-2.97 (m, 2H), 3.18 (d, 1H), 3.42-3.53 (m, 1H), 3.67-3.73 (m, 1H), 4.21-4.26 (m, 1H), 4.29 (d, 1H), 4.27-4.33 (m, 1H), 4.34 (d, 1H), 6.80-6.83 (m, 2H), 6.89 (s, 1H), 7.19-7.24 (m, 4H), 7.26-7.31 (m, 3H), 7.35 (d, 1H).

Examples 3 to 14 listed in the following table can be prepared in analogy to Example 1.

| Example | | Analytical |
|---|---|---|
| No. | Structure | data |
| 3 | 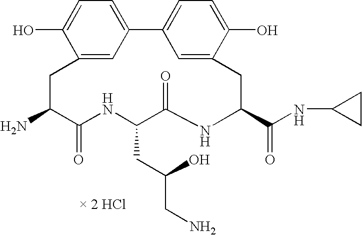 | LC-MS (Method 20): Rₜ = 1.13 min. MS (ESIpos): m/z = 512 (M + H)⁺ |
| 4 | 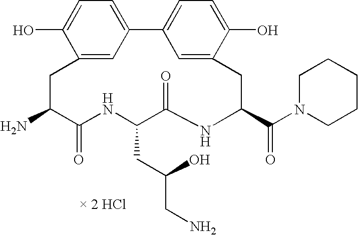 | LC-MS (Method 20): Rₜ = 2.09 min. MS (ESIpos): m/z = 540 (M + H)⁺ |
| 5 | 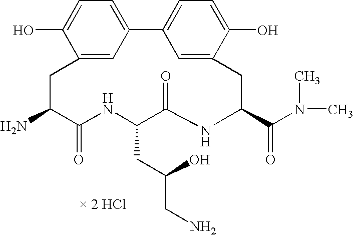 | LC-MS (Method 20): Rₜ = 1.44 min. MS (ESIpos): m/z = 500 (M + H)⁺ |
| 6 | 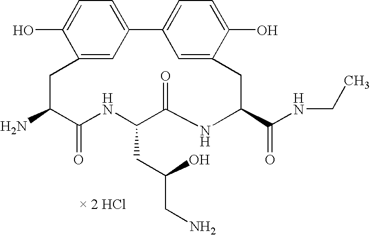 | LC-MS (Method 20): Rₜ = 0.35 min. MS (ESIpos): m/z = 500 (M + H)⁺ |
| 7 | 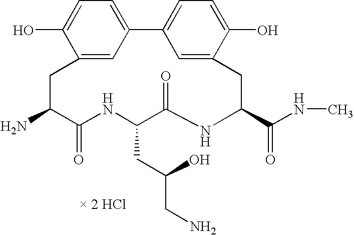 | LC-MS (Method 20): Rₜ = 0.32 min. MS (ESIpos): m/z = 486 (M + H)⁺ |
| 8 | 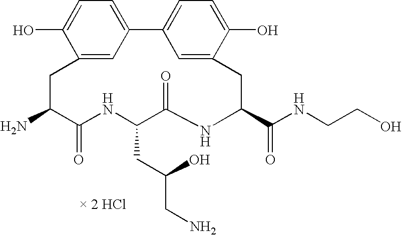 | LC-MS (Method 20): Rₜ = 0.35 min. MS (ESIpos): m/z = 516 (M + H)⁺ |
| 9 | 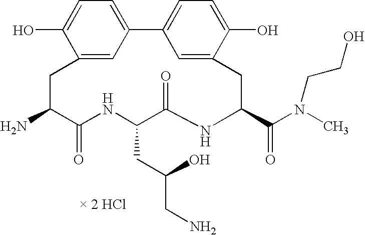 | LC-MS (Method 21): Rₜ = 2.79 min. MS (ESIpos): m/z = 530 (M + H)⁺ |
| 10 | 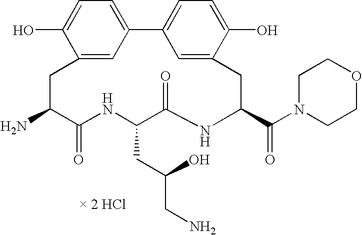 | LC-MS (Method 21): Rₜ = 2.85 min. MS (ESIpos): m/z = 542 (M + H)⁺ |
| 11 | 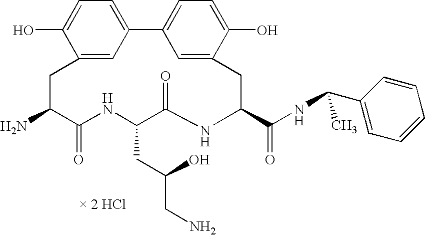 | LC-MS (Method 21): Rₜ = 3.09 min. MS (ESIpos): m/z = 576 (M + H)⁺ |
| 12 | 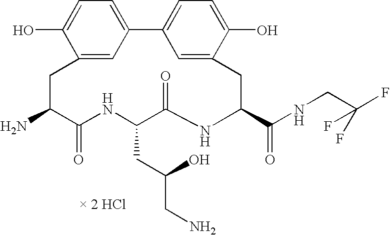 | LC-MS (Method 21): Rₜ = 2.88 min. MS (ESIpos): m/z = 554 (M + H)⁺ |
| 13 | 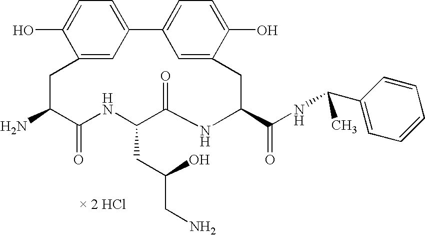 | LC-MS (Method 21): Rₜ = 3.10 min. MS (ESIpos): m/z = 576 (M + H)⁺ |
| 14 | 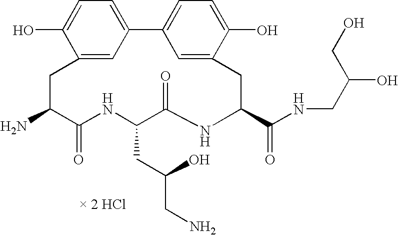 | ¹H-NMR(400 MHz, D₂O): 1.78-1.88(m, 1H), 1.93-2.00(m, 1H), 2.78-2.88(m, 2H), 2.98-3.06(m, 2H), 3.17-3.30(m, 2H), 3.33(d, 1H), 3.42-3.57 (m, 3H), 3.73-3.84 (m, 1H), 4.68-4.82 (m, 2H), 6.86(d, 1H), 6.87(d, 1H), 7.24 (m, 1H), 7.32(d, 1H), 7.40(d, 1H). MS (EI): m/z = 546 (M + H)⁺, 568 (M + Na)⁺ |

### Example 15

### N-{[(8S,11S,14S)-14-Amino-11-[(2R)-3-amino-2-hydroxypropyl]-5,17-dihydroxy-10,13-dioxo-9,12-diazatricyclo[14.3.1.1^{2,6}]henicosa-1(20),2(21),3,5,16,18-hexaen-8-yl]carbonyl}-L-phenylalanine dihydrochloride

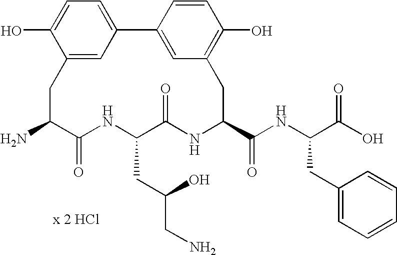

0.02 g (0.02 mmol) of benzyl N-{[(8S,11S,14S)-5,17-bis(benzyloxy)-14-{[(benzyloxy)carbonyl]amino}-11-((2R)-3-{[(benzyloxy)carbonyl]amino}-2-hydroxypropyl)-10,13-dioxo-9,12-diazatricyclo[14.3.1.1^{2,6}]henicosa-1(20),2(21),3,5,16,18-hexaen-8-yl]carbonyl}-L-phenylalaninate are suspended in 6 ml of acetic acid:water:ethanol (4:1:1), and 0.01 g of Pd/C is added. Hydrogenation is carried out under atmospheric pressure with vigorous stirring for 48 h. The reaction solution is filtered. The residue is mixed with 0.25 ml of 0.1 N hydrochloric acid. Concentration in a rotary. evaporator is followed by drying in vacuo. Further purification is achieved by stirring in isopropanol:diethyl ether (1:1).

Yield: 0.0037 g (28% of theory). LC-MS (Method 15): Rₜ=1.27 min. MS (EI): m/z=620 (M+H)⁺.

Examples 16 and 17 listed in the following table can be prepared in analogy to Example 15.

| Example | | Analytical |
|---|---|---|
| No. | Structure | data |
| 16 | 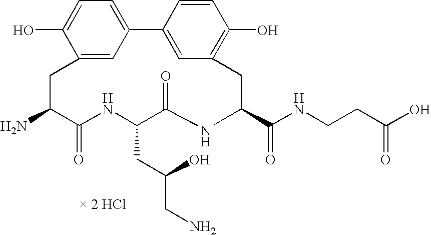 | LC-MS (Method 15): Rₜ = 0.701 min. MS (EI): m/z = 544 (M + H)⁺ |
| 17 | 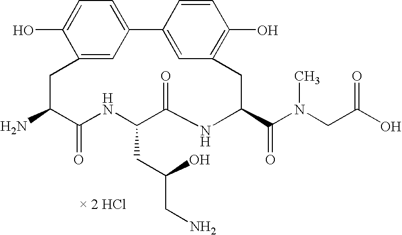 | LC-MS (Method 17): Rₜ = 1.55 min. MS (EI): m/z = 544 (M + H)⁺ |

The L-ornithine-containing amides (Examples 18 to 24) listed in the following table can be prepared starting from (8S,11S,14S)-14-[(tert-butoxycarbonyl)amino-11-[3-[(tert-butoxycarbonyl)amino]propyl}-5,17-dihydroxy-10,13-dioxo-9,12-diazatricyclo[14.3.1.1^{2,6}]henicosa-1(20),2(21),3,5,16,18-hexaene-8-carboxylic acid (Example 83A).

| Example | | Analytical |
|---|---|---|
| No. | Structure | data |
| 18 | 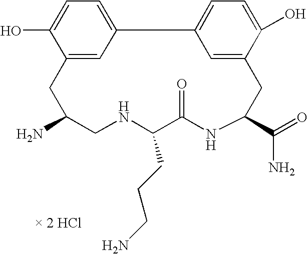 | LC-MS (Method 20): Rₜ = 0.33 min. MS (EI): m/z = 456 (M + H)⁺ |
| 19 | 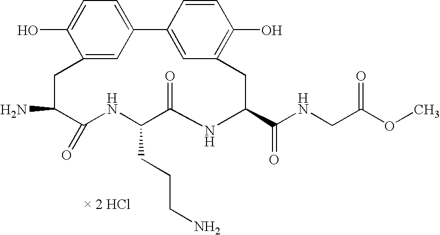 | LC-MS (Method 19): Rₜ = 1.54 min. MS (EI): m/z = 514 (M + H)⁺ |
| 20 | 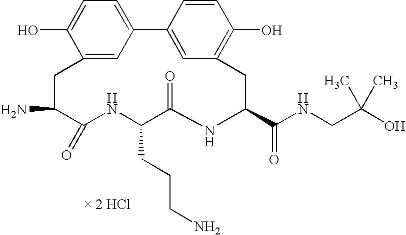 | LC-MS (Method 18): Rₜ = 0.66 min. MS (EI): m/z = 528 (M + H)⁺ |
| 21 | 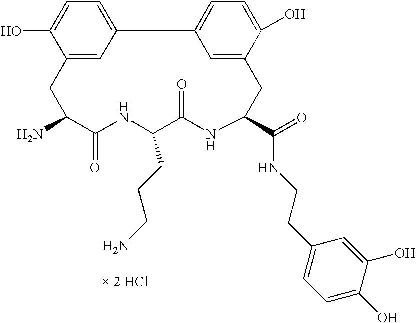 | LC-MS (Method 19): Rₜ = 1.6 min. MS (EI): m/z = 592 (M + H)⁺ |
| 22 | 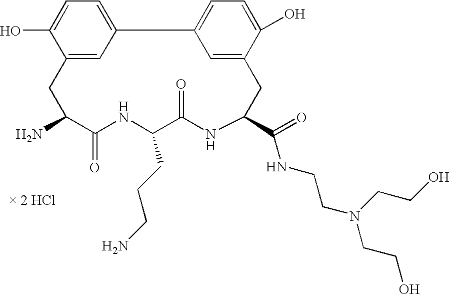 | MS (EI): m/z = 587 (M + H)⁺ |
| 23 | 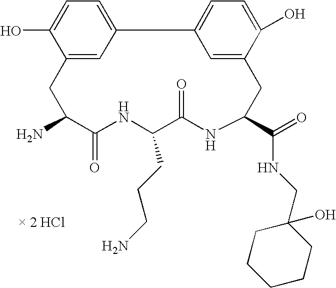 | LC-MS (Method 18): Rₜ = 1.21 min. MS (EI): m/z = 568 (M + H)⁺ |
| 24 | 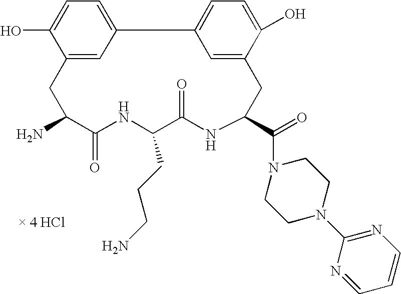 | LC-MS (Method 18): Rₜ = 1.27 min. MS (EI): m/z = 603 (M + H)⁺ |

Examples 25 and 26 listed in the following table can be prepared in analogy to Example 15.

| Example | | Analytical |
|---|---|---|
| No. | Structure | data |
| 25 | 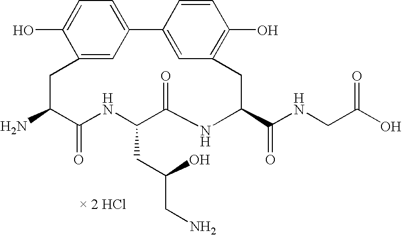 | LC-MS (Method 22): Rₜ = 0.30 min. MS (EI): m/z = 530 (M + H)⁺ |
| 26 | 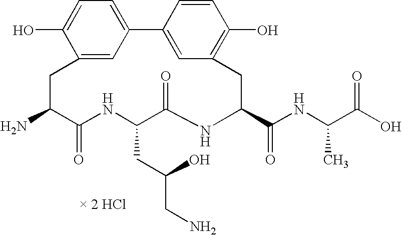 | LC-MS (Method 15): Rₜ = 0.88 min. MS (EI): m/z = 544 (M + H)⁺ |

The L-ornithine-containing amides (Examples 27 to 33) listed in the following table can be prepared starting from (8S,11S,14S)-14-[(tert-butoxycarbonyl)amino-11-[3-[(tert-butoxycarbonyl)amino]propyl}-5,17-dihydroxy-10,13-dioxo-9,12-diazatricyclo[14.3.1.1^{2,6}]henicosa-1(20),2(21),3,5,16,18-hexaene-8-carboxylic acid (Example 83A).

| Example | | Analytical |
|---|---|---|
| No. | Structure | data |
| 27 | 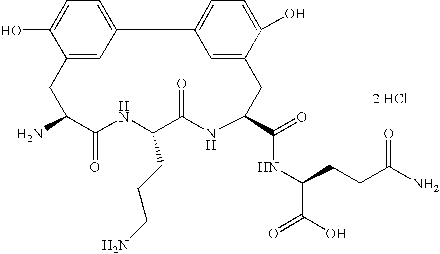 | LC-MS (Method 15): Rₜ = 0.72 min. MS (EI): m/z = 584 (M + H)⁺ |
| 28 | 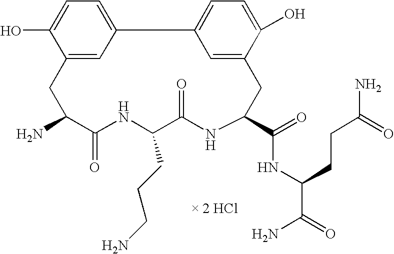 | LC-MS (Method 15): Rₜ = 0.69 min. MS (EI): m/z = 583 (M + H)⁺ |
| 29 | 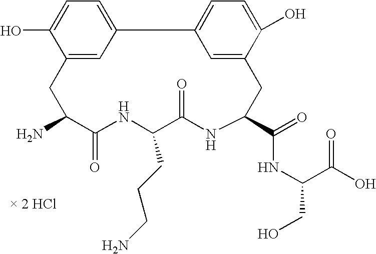 | LC-MS (Method 15): Rₜ = 0.72 min. MS (EI): m/z = 543 (M + H)⁺ |
| 30 | 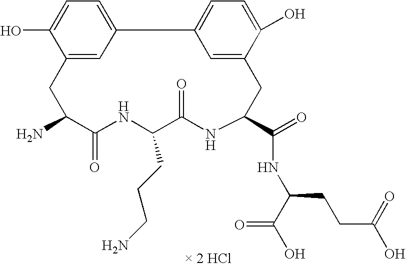 | LC-MS (Method 15): Rₜ = 0.83 min. MS (EI): m/z = 585 (M + H)⁺ |
| 31 | 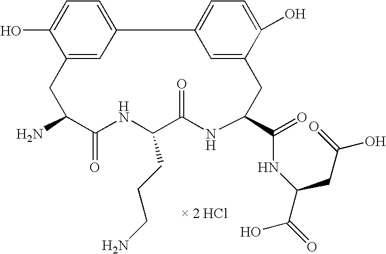 | LC-MS (Method 23): Rₜ = 1.04 min. MS (EI): m/z = 571 (M + H)⁺ |
| 32 | 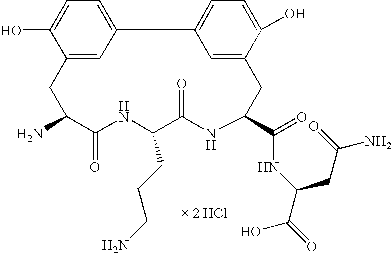 | LC-MS (Method 23): Rₜ = 1.00 min. MS (EI): m/z = 570 (M + H)⁺ |
| 33 | 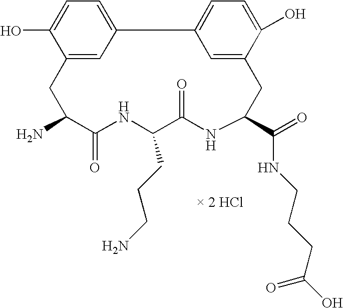 | LC-MS (Method 24): Rₜ = 0.27 min. MS (EI): m/z = 541 (M + H)⁺ |

### Example 34

### (8S,11S,14S)-14-Amino-11-[(2R)-3-amino-2-hydroxypropyl]-5,17-dihydroxy-9-methyl-10,13-dioxo-9,12-diazatricyclo[14.3.1.1^{2,6}]henicosa-1(20),2(21),3,15,16,18-hexaenecarboxamide dihydrochloride

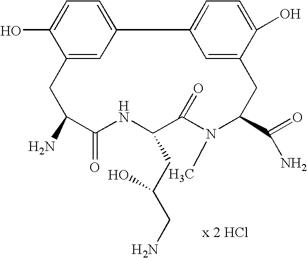

11 mg (0.01 mmol) of the compound from Example 122A are dissolved in 10 ml of glacial acetic acid/ethanol/water (4/1/1), 6 mg of Pd—C (10%) catalyst are added, and the mixture is hydrogenated at room temperature overnight. After removal of the catalyst by filtration, the residue is evaporated to dryness in vacuo, 0.1 N hydrochloric acid is added, and the mixture is again evaporated to dryness.

Yield: 7 mg (96% of theory). MS (EI): m/z=485 (M+H)⁺.

Example 35 detailed in the following table can be prepared in analogy to the method for Example 34:

| Example | | Analytical |
|---|---|---|
| No. | Structure | data |
| 35 | 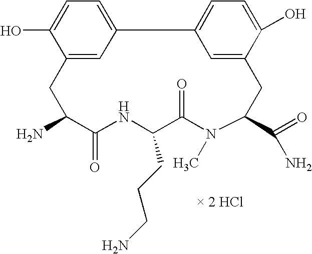 | LC-MS (Method 22): Rₜ = 1.46 min. MS (EI): m/z = 469 (M + H)⁺ |

Examples 36 and 37 listed in the following table can be prepared in analogy to Example 1.

| Example | | Analytical |
|---|---|---|
| No. | Structure | data |
| 36 | 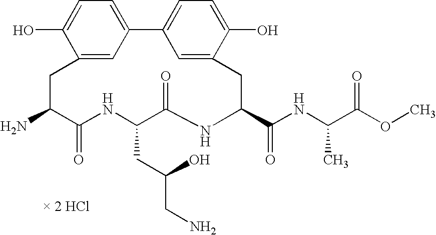 | LC-MS (Method 15): Rₜ = 1.52 min. MS (EI): m/z = 558 (M + H)⁺ |
| 37 | 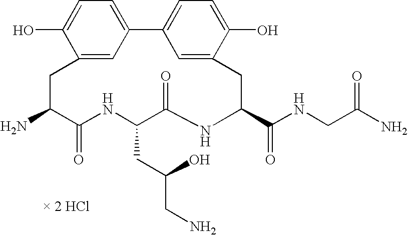 | LC-MS (Method 24): Rₜ = 0.42 min. MS (EI): m/z = 529 (M + H)⁺ |

Examples 38 to 40 listed in the following table can be prepared in analogy to Example 15.

| Example | | Analytical |
|---|---|---|
| No. | Structure | data |
| 38 | 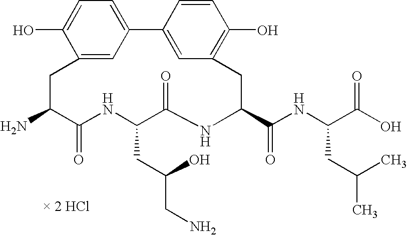 | LC-MS (Method 23): Rₜ = 0.95 min. MS (EI): m/z = 586 (M + H)⁺ |
| 39 | 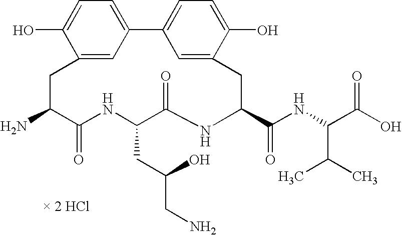 | LC-MS (Method 24): Rₜ = 0.80 min. MS (EI): m/z = 572 (M + H)⁺ |
| 40 | 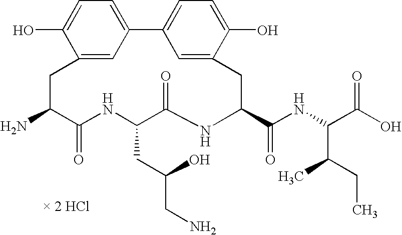 | LC-MS (Method 24): Rₜ = 0.94 min. MS (EI): m/z = 586 (M + H)⁺ |

A. Assessment of the Physiological Activity

The in vitro effect of the compounds of the invention can be shown in the following assays:

In vitro Transcription-translation with E. coli Extracts

An S30 extract is prepared by harvesting logarithmically growing Escherichia coli MRE 600 (M. Müller; University Freiburg), washing and employing them as described for the in vitro transcription-translation assay (Müller, M. and Blobel, G. Proc Natl Acad Sci USA (1984) 81, pp. 7421-7425).

1 μl of cAMP (11.25 mg/ml) are additionally added per 50 μl of reaction mix to the reaction mix for the in vitro transcription-translation assay. The assay mixture amounts to 105 μl, with 5 μl of the substance to be tested being introduced in 5% strength DMSO. 1 μg/100 μl of mixture of the plasmid pBESTLuc (Promega, Germany) are used as transcription template. After incubation at 30° C. for 60 min, 50 μl of luciferin solution (20 mM tricine, 2.67 mM MgSO4, 0.1 mM EDTA, 33.3 mM DTT pH 7.8, 270 μM CoA, 470 μM luciferin, 530 μM ATP) are added, and the resulting bioluminescence is measured in a luminometer for 1 minute. The IC₅₀ is indicated by the concentration of an inhibitor which leads to 50% inhibition of the translation of firefly luciferase.

In vitro Transcription-translation with S. aureus Extracts

Construction of an S. aureus Luciferase Reporter Plasmid

A reporter plasmid which can be used in an in vitro transcription-translation assay for S. aureus is constructed by using the plasmid pBESTluc (Promega Corporation, USA). The E. coli tac promoter present in this plasmid in front of the firefly luciferase is replaced by the capAl promoter with appropriate Shine-Dalgarno sequence from S. aureus. The primers CAPFor 5′-CGGCCAAGCTTACTCGGATCCAGAGTTTGCAAAATATACAGGGGATTATATATAATGGAAAACAAGAA AGGAAAATAGGAGGTTTATATGGAAGACGCCA-3′ and CAPRev 5′-GTCATCGTCGGGAAGACCTG-3′ are used for this. The primer CAPFor contains the capAl promoter, the ribosome binding site and the 5′ region of the luciferase gene. After PCR using pBESTluc as template it is possible to isolate a PCR product which contains the firefly luciferase gene with the fused capAl promoter. This is, after restriction with Clal and HindIII, ligated into the vector pBESTluc which has likewise been digested with ClaI and HindIII. The resulting plasmid pla is able to replicate in E. coli and be used as template in the S. aureus in vitro transcription-translation assay.

Preparation of S30 Extracts from S. aureus

Six liters of BHI medium are inoculated with a 250 ml overnight culture of an S. aureus strain and allowed to grow at 37° C. until the OD600 nm is 2-4. The cells are harvested by centrifugation and washed in 500 ml of cold buffer A (10 mM Tris acetate, pH 8.0, 14 mM Mg acetate, 1 mM DTT, 1 M KCl). After renewed centrifugation, the cells are washed in 250 ml of cold buffer A with 50 mM KCl, and the resulting pellets are frozen at −20° C. for 60 min. The pellets are thawed on ice in 30 to 60 min and taken up to a total volume of 99 ml in buffer B (10 mM Tris acetate, pH 8.0, 20 mM Mg acetate, 1 mM DTT, 50 mM KCl). 1.5 ml portions of lysostaphin (0.8 mg/ml) in buffer B are introduced into 3 precooled centrifuge cups and each mixed with 33 ml of the cell suspension. The samples are incubated at 37° C., shaking occasionally, for 45 to 60 min, before 150 μl of a 0.5 M DTT solution are added. The lyzed cells are centrifuged at 30 000×g and 4° C. for 30 min. The cell pellet is taken up in buffer B and then centrifuged again under the same conditions, and the collected supernatants are combined. The supernatants are centrifuged again under the same conditions, and 0.25 volume of buffer C (670 mM Tris acetate, pH 8.0, 20 mM Mg acetate, 7 mM Na₃ phosphenolpyruvate, 7 mM DTT, 5.5 mM ATP, 70 μM amino acids (complete from Promega), 75 μg of pyruvate kinase (Sigma, Germany)/ml are added to the upper 2/3 of the supernatant. The samples are incubated at 37° C. for 30 min. The supernatants are dialyzed against 2 l of dialysis buffer (10 mM Tris acetate, pH 8.0, 14 mM Mg acetate, 1 mM DTT, 60 mM K acetate) in a dialysis tube with a 3500 Da cut-off with one buffer change at 4° C. overnight. The dialysate is concentrated to a protein concentration of about 10 mg/ml by covering the dialysis tube with cold PEG 8000 powder (Sigma, Germany) at 4° C. The S30 extracts can be stored in aliquots at −70° C.

Determination of the IC₅₀ in the S. aureus in vitro Transcription-translation Assay

Inhibition of protein biosynthesis of the compounds can be shown in an in vitro transcription-translation assay. The assay is based on the cell-free transcription and translation of firefly luciferase using the reporter plasmid pla as template and cell-free S30 extracts obtained from S. aureus. The activity of the resulting luciferase can be detected by luminescence measurement.

The amount of S30 extract or plasmid pla to be employed must be tested anew for each preparation in order to ensure an optimal concentration in the assay. 3 μl of the substance to be tested, dissolved in 5% DMSO, are introduced into an MTP. Then 10 μl of a suitably concentrated plasmid solution pla are added. Then 46 μl of a mixture of 23 μl of premix (500 mM K acetate, 87.5 mM Tris acetate, pH 8.0, 67.5 mM ammonium acetate, 5 mM DTT, 50 μg of folic acid/ml, 87.5 mg of PEG 8000/ml, 5 mM ATP, 1.25 mM each NTP, 20 μM each amino acid, 50 mM PEP (Na₃ salt), 2.5 mM cAMP, 250 μg of each E. coli tRNA/ml) and 23 μl of a suitable amount of S. aureus S30 extract are added and mixed. After incubation at 30° C. for 60 min, 50 μl of luciferin solution (20 mM tricine, 2.67 mM MgSO₄, 0.1 mM EDTA, 33.3 mM DTT pH 7.8, 270 μM CoA, 470 μM luciferin, 530 μM ATP) are, and the resulting bioluminescence is measured in a luminometer for 1 min. The IC₅₀ is indicated as the concentration of an inhibitor which leads to 50% inhibition of the translation of firefly luciferase.

Determination of the Minimum Inhibitory Concentration (MIC):

The minimum inhibitory concentration (MIC) is the minimum concentration of an antibiotic with which the growth of a test microbe is inhibited over 18-24 h. The inhibitor concentration can in these cases be determined by standard microbiological methods (see, for example, The National Committee for Clinical Laboratory Standards. Methods for dilution antimicrobial susceptibility tests for bacteria that grow aerobically; approved standard-fifth edition. NCCLS document M7-A5 [ISBN 1-56238-394-9]. NCCLS, 940 West Valley Road, Suite 1400, Wayne, Pa. 19087-1898 USA, 2000). The MIC of the compounds of the invention is determined in the liquid dilution test on the 96-well microtiter plate scale. The bacterial microbes are cultivated in a minimal medium (18.5 mM Na₂HPO₄, 5.7 mM KH₂PO₄, 9.3 mM NH₄Cl, 2.8 mM MgSO₄, 17.1 mM NaCl, 0.033 μg/ml thiamine hydrochloride, 1.2 μg/ml nicotinic acid, 0.003 μg/ml biotin, 1% glucose, 25 μg/ml of each proteinogenic amino acid with the exception of phenylalanine; [H.-P. Kroll; unpublished]) with addition of 0.4% BH broth (test medium). In the case of Enterococcus faecalis ICB 27159, heat-inactivated fetal calf serum (FCS; GibcoBRL, Germany) is added to the test medium in a final concentration of 10%. Overnight cultures of the test microbes are diluted to an OD₅₇₈ of 0.001 (to 0.01 in the case of Enterococci) in fresh test medium, and incubated 1:1 with dilutions of the test substances (1:2 dilution steps) in test medium (150 μl final volume). The cultures are incubated at 37° C. for 18-24 hours; Enterococci in the presence of 5% CO₂.

The lowest substance concentration in each case at which bacterial growth was no longer visible is defined as the MIC. The MIC values in μM of some compounds of the invention for a series of test microbes are listed by way of example in the table below. The compounds show a graded antibacterial effect against most of the test microbes.

TABLE A

| | | | | | | IC50 | IC50 | IC50 |
|---|---|---|---|---|---|---|---|---|
| | MIC | MIC | MIC | MIC | MIC | E. coli | S. aureus | S. aureus |
| Ex. | S. aureus | S. aureus | S. aureus | E. faecalis | B. catarrhalis | MRE600 | 133 | RN4220 |
| No. | 133 | RN4220 | 25701 | ICB27159 | M3 | Translation | Translation | Translation |
| 1 | 0.2 | 0.1 | 6.25 | 6.25 | 1.56 | 0.15 | 0.9 | 0.5 |
| 2 | 25 | 12.5 | 50 | 25 | 25 | 0.55 | 1.3-4.5 | 3.4 |
| 37 | 0.8 | — | — | — | — | — | 0.5 | — |

All concentration data in μM.

Systemic Infection with S. aureus 133

The suitability of the compounds of the invention for treating bacterial infections can be shown in various animal models. For this purpose, the animals are generally infected with a suitable virulent microbe and then treated with the compound to be tested, which is in a formulation which is adopted to the particular therapy model. The suitability of the compounds of the invention can be demonstrated specifically for the treatment of bacterial infections in a mouse sepsis model after infection with S. aureus.

For this purpose, S. aureus 133 cells are cultured overnight in BH broth (Oxoid, Germany). The overnight culture is diluted 1:100 in fresh BH broth and expanded for 3 hours. The bacteria which are in the logarithmic phase of growth are centrifuged and washed 2× with buffered physiological saline solution. A cell suspension in saline solution with an extinction of 50 units is then adjusted in a photometer (Dr. Lange LP 2W). After a dilution step (1:15), this suspension is mixed 1:1 with a 10% strength mucine suspension. 0.2 ml of this infection solution is administered i.p. per 20 g of mouse. This corresponds to a cell count of about 1-2×10E6 microbes/mouse. The i.v. therapy takes place 30 minutes after the infection. Female CFW1 mice are used for the infection test. The survival of the animals is recorded for 6 days. The animal model is adjusted so that untreated animals die within 24 h after the infection. It was possible to demonstrate in this model a therapeutic effect of ED100=1.25 mg/kg for the compound of Example 2.

B. Exemplary Embodiments of Pharmaceutical Compositions

The compounds of the invention can be converted into pharmaceutical preparations in the following ways:

Tablet:

Composition:

100 mg of the compound of Example 1, 50 mg of lactose (monohydrate), 50 mg of corn starch (native), 10 mg of polyvinylpyrolidone (PVP 25) (from BASF, Ludwigshafen, Germany) and 2 mg of magnesium stearate. Tablet weight 212 mg, diameter 8 mm, radius of curvature 12 mm.

Production:

A mixture of active ingredient, lactose and starch is granulated with a 5% strength solution (m/m) of the PVP in water. The granules are dried and then mixed with the magnesium stearate for 5 min. This mixture is compressed with a conventional tablet press (see above for format of the tablet). A compressive force of 15 kN is used as guideline for the compression.

Suspension which can be Administered Orally:

Composition:

1000 mg of the compound of Example 1, 1000 mg of ethanol (96%), 400 mg of Rhodigel (xanthan gum from FMC, Pennsylvania, USA) and 99 g of water. 10 ml of oral suspension correspond to a single dose of 100 mg of the compound of the invention.

Production:

The Rhodigel is suspended in ethanol, and the active ingredient is added to the suspension. The water is added with stirring. The mixture is stirred for about 6 h until the swelling of the Rhodigel is complete.

1. A compound of the formula 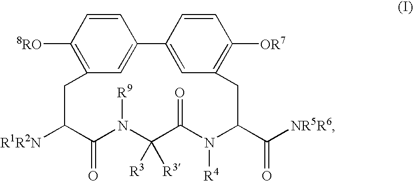
in which
R¹ is hydrogen, alkyl, aryl, heteroaryl, heterocyclyl, alkylcarbonyl, arylcarbonyl, heterocyclylcarbonyl, heteroarylcarbonyl, alkoxycarbonyl, aminocarbonyl, alkylaminocarbonyl, dialkylaminocarbonyl, alkylsulfonyl, arylsulfonyl, heterocyclylsulfonyl, heteroarylsulfonyl or a carbonyl-linked amino acid residue,
where R¹ apart from hydrogen may be substituted by 0, 1, 2 or 3 substituents R¹⁻¹, where the substituents R¹⁻¹ are selected independently of one another from the group consisting of halogen, alkyl, trifluoromethyl, trifluoromethoxy, nitro, cyano, amino, alkylamino, dialkylamino, cycloalkyl, aryl, heteroaryl, heterocyclyl, hydroxy, alkoxy and carboxyl,
R² is hydrogen or alkyl,
where R² apart from hydrogen may be substituted by 0, 1, 2 or 3 substituents R²⁻¹, where the substituents R²⁻¹ are selected independently of one another from the group consisting of halogen, amino, alkylamino and dialkylamino,
or
R¹ and R² together with the nitrogen atom to which they are bonded form a heterocycle which may be substituted by 0, 1 or 2 substituents R¹⁻², where the substituents R¹⁻² are selected independently of one another from the group consisting of halogen, trifluoromethyl, amino, alkylamino, dialkylamino, cycloalkyl, aryl, heteroaryl, heterocyclyl, hydroxy, alkoxy, carboxyl, alkoxycarbonyl and aminocarbonyl,
R³ is hydrogen, alkyl or the side group of an amino acid, in which alkyl may be substituted by 0, 1, 2 or 3 substituents R³⁻¹, where the substituents R³⁻¹ are selected independently of one another from the group consisting of trifluoromethyl, nitro, amino, alkylamino, dialkylamino, cycloalkyl, aryl, heteroaryl, heterocyclyl, hydroxy, alkoxy, carboxyl, alkoxycarbonyl, aminocarbonyl, alkylaminocarbonyl, dialkylaminocarbonyl, guanidino and amidino,
in which cycloalkyl, aryl, heteroaryl and heterocyclyl may be substituted by 0, 1 or 2 substituents R³⁻², where the substituents R³⁻² are selected independently of one another from the group consisting of halogen, alkyl, trifluoromethyl and amino,
and in which free amino groups in the side group of the amino acid may be substituted by alkyl, alkenyl, alkynyl, cycloalkyl, aryl, heteroaryl, heterocyclyl, alkylcarbonyl, arylcarbonyl, heteroarylcarbonyl, heterocyclylcarbonyl, alkoxycarbonyl, aminocarbonyl, alkylaminocarbonyl, dialkylaminocarbonyl, arylaminocarbonyl, alkylsulfonyl, arylsulfonyl, heterocyclylsulfonyl or heteroarylsulfonyl,
R^{3′} is hydrogen, C₁-C₆-alkyl or C₃-C₈-cycloalkyl,
R⁴ is hydrogen, C₁-C₆-alkyl or C₃-C₈-cycloalkyl,
R⁵ is hydrogen, alkyl, alkenyl, cycloalkyl, aryl, heteroaryl, heterocyclyl or an amine-linked amino acid residue,
where R⁵ may be substituted by 0, 1, 2 or 3 substituents R⁵⁻¹, where the substituents R⁵⁻¹ are selected independently of one another from the group consisting of halogen, alkyl, trifluoromethyl, trifluoromethoxy, nitro, cyano, amino, alkylamino, dialkylamino, cycloalkyl, aryl, heteroaryl, heterocyclyl, hydroxy, alkoxy, carboxyl, alkoxycarbonyl, aminocarbonyl, alkylaminocarbonyl, dialkylaminocarbonyl, aminosulfonyl, alkylaminosulfonyl, dialkylaminosulfonyl, arylaminosulfonyl, heterocyclylaminosulfonyl, heteroarylaminosulfonyl, aminocarbonylamino, hydroxycarbonylamino and alkoxycarbonylamino,
in which alkyl, alkylamino, dialkylamino, cycloalkyl, aryl, heteroaryl and heterocyclyl may be substituted by 0, 1, 2 or 3 substituents R⁵⁻², where the substituents R⁵⁻² are selected independently of one another from the group consisting of hydroxy, amino, carboxyl and aminocarbonyl,
R⁶ is hydrogen, alkyl or cycloalkyl,
or
R⁵ and R⁶ together with the nitrogen atom to which they are bonded form a heterocycle which may be substituted by 0, 1, 2 or 3 substituents R⁵⁻⁶, where the substituents R⁵⁻⁶ are selected independently of one another from the group consisting of halogen, alkyl, trifluoromethyl, nitro, amino, alkylamino, dialkylamino, cycloalkyl, aryl, halogenated aryl, heteroaryl, heterocyclyl, hydroxy, alkoxy, carboxyl, alkylcarbonyl, alkoxycarbonyl, aminocarbonyl, alkylaminocarbonyl and dialkylaminocarbonyl,
R⁷ is hydrogen, C₁-C₆-alkyl, alkylcarbonyl or C₃-C₈-cycloalkyl,
R⁸ is hydrogen or C₁-C₆-alkyl, and
R⁹ is hydrogen or C₁-C₆-alkyl,
and one of the salts thereof, or one of the solvates thereof and one of the solvates of the salts thereof.

2. A compound as claimed in claim 1, characterized in that it corresponds to the formula 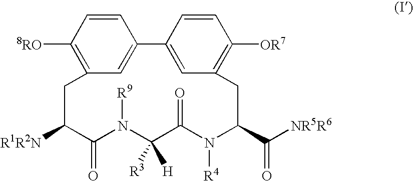
in which R¹ to R⁹ have the same meaning as in formula (I).

3. A compound as claimed in claim 1 or 2, characterized in that
R¹ is hydrogen, alkyl or alkylcarbonyl,
R² is hydrogen,
R³ is alkyl or the side group of an amino acid, in which alkyl may be substituted by 0, 1, 2 or 3 substituents R³⁻¹, where the substituents R³⁻¹ are selected independently of one another from the group consisting of trifluoromethyl, nitro, amino, alkylamino, dialkylamino, cycloalkyl, aryl, heteroaryl, heterocyclyl, hydroxy, alkoxy, carboxyl, alkoxycarbonyl, aminocarbonyl, alkylaminocarbonyl, dialkylaminocarbonyl, guanidino and amidino,
in which cycloalkyl, aryl, heteroaryl and heterocyclyl may be substituted by 0, 1 or 2 substituents R³⁻², where the substituents R³⁻² are selected independently of one another from the group consisting of halogen, alkyl, trifluoromethyl and amino,
and in which free amino groups in the side group of the amino acid may be substituted by alkyl,
R^{3′} is hydrogen, C₁-C₆-alkyl or C₃-C₈-cycloalkyl,
R⁴ is hydrogen, C₁-C₆-alkyl or C₃-C8-cycloalkyl,
R⁵ is hydrogen, alkyl, alkenyl, cycloalkyl, aryl, heteroaryl, heterocyclyl or an amine-linked amino acid residue,
where alkyl, alkenyl, cycloalkyl, aryl, heteroaryl and heterocyclyl may be substituted by 0, 1, 2 or 3 substituents R⁵⁻¹, where the substituents R⁵⁻¹ are selected independently of one another from the group consisting of halogen, alkyl, trifluoromethyl, trifluoromethoxy, nitro, cyano, amino, alkylamino, dialkylamino, cycloalkyl, aryl, heteroaryl, heterocyclyl, hydroxy, alkoxy, carboxyl, alkoxycarbonyl, aminocarbonyl, alkylaminocarbonyl and dialkylaminocarbonyl,
in which alkyl, alkylamino, dialkylamino, cycloalkyl, aryl, heteroaryl and heterocyclyl may be substituted by 0, 1, 2 or 3 substituents R⁵⁻²,
where the substituents R⁵⁻² are selected independently of one another from the group consisting of hydroxy, amino, carboxyl and aminocarbonyl,
R⁶ is hydrogen, alkyl or cycloalkyl,
or
R⁵ and R⁶ together with the nitrogen atom to which they are bonded form a heterocycle which may be substituted by 0, 1, 2 or 3 substituents R⁵⁻⁶, where the substituents R⁵⁻⁶ are selected independently of one another from the group consisting of halogen, alkyl, amino, alkylamino, dialkylamino, hydroxy, alkoxy, carboxyl, alkoxycarbonyl, aminocarbonyl, alkylaminocarbonyl and dialkylaminocarbonyl,
R⁷ is hydrogen, C₁-C₆-alkyl, alkylcarbonyl or C₃-C₈-cycloalkyl,
R⁸ is hydrogen,
and
R⁹ is hydrogen.

4. A compound as claimed in any of claims 1 to 3, characterized in that
R¹ is hydrogen,
R² is hydrogen,
R³ is aminocarbonylmethyl, 3-aminoprop-1-yl, 2-hydroxy-3-aminoprop-1-yl, 1-hydroxy-3-aminoprop-1-yl, 3-guanidinoprop-1-yl, 2-aminocarbonylethyl, 2-hydroxycarbonylethyl, 4-aminobut-1-yl, hydroxymethyl, 2-hydroxyethyl, 2-aminoethyl, 4-amino-3-hydroxybut-1-yl or (1-piperidin-3-yl)methyl,
R^{3′} is hydrogen,
R⁴ is hydrogen, methyl, ethyl, isopropyl or cyclopropyl,
R⁵ is hydrogen, C₁-C₆-alkyl or C₃-C₈-cycloalkyl,
where alkyl and cycloalkyl may be substituted by 0, 1, 2 or 3 substituents R⁵⁻¹, where the substituents R⁵⁻¹ are selected independently of one another from the group consisting of halogen, C₁-C₆-alkyl, trifluoromethyl, trifluoromethoxy, amino, C₁-C₆-alkylamino, C₁-C₆-dialkylamino, C₃-C₈-cycloalkyl, C₆-C₁₀-aryl, 5- to 10-membered heteroaryl, 5- to 7-membered heterocyclyl, hydroxy, alkoxy, carboxyl, C₁-C₆-alkoxycarbonyl, aminocarbonyl, C₁-C₆-alkylaminocarbonyl and C₁-C₆-dialkylaminocarbonyl,
R⁶ is hydrogen or methyl,
or
R⁵ and R⁶ together with the nitrogen atom to which they are bonded form a piperidinyl or morpholinyl,
R⁷ is hydrogen,
R⁸ is hydrogen,
and
R⁹ is hydrogen.

5. A compound as claimed in any of claims 1 to 4, characterized in that
R¹ is hydrogen,
R² is hydrogen,
R³ is 3-aminoprop-1-yl or 2-hydroxy-3-aminoprop-1-yl,
R^{3′} is hydrogen,
R⁴ is hydrogen or methyl,
R⁵ is hydrogen, C₁-C₆-alkyl or cyclopropyl,
where alkyl may be substituted by 0, 1, 2 or 3 substituents R⁵⁻¹, where the substituents R⁵⁻¹ are selected independently of one another from the group consisting of trifluoromethyl, amino, hydroxy, carboxyl, aminocarbonyl and phenyl,
R⁶ is hydrogen or methyl,
R⁷ is hydrogen,
R⁸ is hydrogen
and
R⁹ is hydrogen.

6. A compound as claimed in any of claims 1 to 3, characterized in that R¹ is hydrogen.

7. A compound as claimed in any of claims 1, 2 and 6, characterized in that R² is hydrogen.

8. A compound as claimed in any of claims 1 to 4, 6 and 7, characterized in that R³ is 3-aminoprop-1-yl or 2-hydroxy-3-aminoprop-1-yl.

9. A compound as claimed in any of claims 1 to 3 or 6 to 8, characterized in that R^{3′} is hydrogen.

10. A compound as claimed in any of claims 1 to 4 or 6 to 9, characterized in that R⁴ is hydrogen or methyl.

11. A compound as claimed in any of claims 1 to 4 or 6 to 10, characterized in that R⁵ is hydrogen, C₁-C₆-alkyl or cyclopropyl,
where alkyl may be substituted by 0, 1, 2 or 3 substituents R⁵⁻¹, where the substituents R⁵⁻¹ are selected independently of one another from the group consisting of trifluoromethyl, amino, hydroxy, carboxyl, aminocarbonyl and phenyl.

12. A compound as claimed in any of claims 1 to 3 or 6 to 11, characterized in that R⁶ is hydrogen or methyl.

13. A compound as claimed in any of claims 1 to 4 or 6 to 12, characterized in that R⁵ and R⁶ together with the nitrogen atom to which they are bonded form a piperidinyl or morpholinyl.

14. A compound as claimed in any of claims 1 to 3 or 6 to 13, characterized in that R⁷ is hydrogen.

15. A compound as claimed in any of claims 1, 2, 6 to 14, characterized in that R⁸ is hydrogen.

16. A compound as claimed in any of claims 1, 2, 6 to 15, characterized in that R⁹ is hydrogen.

17. A process for preparing a compound of the formula (I) as claimed in claim 1, characterized in that a compound of the formula 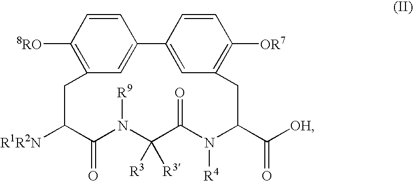
in which R¹ to R⁴ and R⁷ to R⁹ have the meaning indicated in claim 1,
is reacted with a compound of the formula
H—NR⁵R⁶ (III),
in which R⁵ and R⁶ have the meaning indicated in claim 1.

18. A compound as claimed in any of claims 1 to 16 for the treatment and/or prophylaxis of diseases.

19. A medicament comprising at least one compound as claimed in any of claims 1 to 16 in combination with at least one pharmaceutically suitable, pharmaceutically acceptable carrier or other excipients.

20. The use of a compound as claimed in any of claims 1 to 16 for producing a medicament for the treatment and/or prophylaxis of bacterial diseases.

21. A medicament as claimed in claim 19 for the treatment and/or prophylaxis of bacterial infections.

22. A method for controlling bacterial infections in humans and animals by administration of an antibacterially effective amount of at least one compound as claimed in any of claims 1 to 16.
